SEQUENCE LISTING (279 sequences)

SEQ ID NO: 1 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 2 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 3 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 4 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 5 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 6 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 7 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 8 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 9 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 10 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 11 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 12 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 13 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 14 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 15 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 16 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 17 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 18 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 19 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 20 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 21 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 22 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 23 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 24 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 25 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 26 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 27 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 28 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 29 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 30 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 31 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 32 - DNA, 999 bp, Arabidopsis thaliana
  misc_feature (1)..(999): Ceres Promoter YP0019
gatataagtagaatcattttttgccgccgtttctcgctaacacaccgaaaactgaatcaaatctcctagctcttctacgcaaaatcgagtgcatcgacaatggcggaacgtggtgtcgaacgtggtggagatcgcggcgatttcggacgtggattcggtggtcgcggcggtggaagaggtggtccgagaggtcgtggtcgccgtgcaggtcgtgctccagaggaggagaaatgggtgccagtgactaagcttggtcgtctcgtaaaggaaggtaagatcacaaagattgagcagatctacctccattctctcccagtcaaggagtaccagatcatagatttactcgtcggtccttcattgaaagacgaagtgatgaaaatcatgccggttcaaaaacaaaccagagccggtcagagaacgagattcaaggccttcatcgtcgtcggagatagtaacggtcacgtcggattaggagtcaaatgctccaaggaagttgcgacggcgatcagaggcgcgatcattctcgcgaaattgtctgtggttccgatacgaagaggttattggggtaacaagattggaaaaccacatacggttccgtgtaaggtaaccgggaaatgtggatctgttactgtacgtatggttccagctccgagaggttctggtattgtggcggctagagttcctaagaaggttcttcaattcgctggaattgatgatgtctttacttcttctagaggatccaccaaaactcttggaaacttcgtcaaggtatgtactttcacaatggctgttttggtttgatgaactctgaattaggcagtgaaaaagtaatcattaccagttaagtgaatttatattgaagattaggatttagctgattgtattggtttgagcatgtgagtttgtgtttaagattgcttgaattgaaatgctttaggttgtttgattacgctaaattctgactaatgtaattcaaattgttgttgtttttttttggtc

SEQ ID NO: 33 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 34 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 35 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 36 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 37 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 38 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 39 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 40 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 41 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 42 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 43 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 44 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 45 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 46 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 47 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 48 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 49 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 50 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 51 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 52 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 53 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 54 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 55 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 56 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 57 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 58 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 59 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 60 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 61 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 62 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 63 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 64 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 65 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 66 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 67 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 68 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 69 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 70 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 71 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 72 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 73 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 74 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 75 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 76 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 77 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 78 - DNA, 1024 bp, Arabidopsis thaliana
  misc_feature (1)..(1024): Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtca

SEQ ID NO: 79 - DNA, 857 bp, Arabidopsis thaliana
  misc_feature (1)..(857): Ceres Promoter PD1367
ttggaattaattctgcggccatggggctgcaggaattcgatggcccgatcggccacagttttcttttctcatcttacaacaagtttccaggaggatagagacataaacgaagctcnggattgtatcgttctttttnagcttttattcacatccngaaangtcctgtangttntangattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttnnctcctnctttcacttcatcacttggcttgtgaattcattaattncctgcgaaggccntgcagggccatcaccactgcagtggaacaatgaagactaatctttttctctttctcatcttttcacttctcctatcattatcctcggccgaattcagtaaaggagaagaacttttcactg

SEQ ID NO: 80 - DNA, 607 bp, Arabidopsis thaliana
  misc_feature (1)..(607): Ceres CLONE ID no. 120446
aaatcattattaaacctttcataatacacatatatcatatatattgctactactcacgatcaaagacttttaaaccttatttttatatatttgtagttttggatatggcaaagacacgtcgtgtaatttaccttttccttactatagtactccttttctgcgaactcatcgacgaggcgcaaggtagccgttttaggtgtcatcactcagaagattattcatgtaagaaacgttcaagccatcaccatcatcatcatcatcatcatcatcatcatcagcagcagcagcaccatcacaaggacactcccccagaggagcttcaaggaagcatcaagacgaggaggagcaaggatatttatggtctcaatgctttccggtctactgaaccaggtcacagccctggtgttggccacttgatcaagacctgatagaaaattgaatttattaatctggatttctcctactctttttcccttccctacattgttccatcaaccaaaggttgttcactttggagacctaattcagttatgcatgagtcatgctgtttttctcatattgtaaatagtataatttggtttggtctcttaataattgttgtcgcttc

SEQ ID NO: 81 - protein, 107 aa, Arabidopsis thaliana
  misc_feature (1)..(107): Ceres CLONE ID no. 120446
MAKTRRVIYLFLTIVLLFCELIDEAQGSRFRCHHSEDYSCKKRSSHHHHHHHHHHHHQQQQHHHKDTPPEELQGSIKTRRSKDIYGLNAFRSTEPGHSPGVGHLIKT

SEQ ID NO: 82 - DNA, 593 bp, Arabidopsis thaliana
  misc_feature (1)..(593): Ceres CLONE ID no. 11852
agatccgaatcctagttcgatcttgatcttcccggcggagtaaccaaaattccgtaaatggcgtcaaggagaagtgtacggtatgcacagcttccaggagatgaagatgatgaaggctatggaaatggtgttggtgaaaggagagatttcgatcctcggttcgattattcaccgaaagcatttgatagagttccatggaaatctatagcattagctgtgtttcttttgtttcttggttgcttgcttcttcttttgtcgttttttatcttcattggtcacatggaaggagatagctctcaaggttatgcgttacttgttcttgggatccttacttttctccctggtttctacgagacccgaattgcttactactcatggagaggagctgaagggtaccgttttgcagccattccctcttactgaactcaagtaactggagaagtaaagaatattgtgcatagaaaaagttttagagattgtaatccaaaaagtactactatacttaagctgtttcatgaatctactgtgtagaatctcctctagttgagtttagacagataaaagcttggattatagagatacagatactacat

SEQ ID NO: 83 - protein, 121 aa, Arabidopsis thaliana
  misc_feature (1)..(121): Ceres CLONE ID no. 11852
MASRRSVRYAQLPGDEDDEGYGNGVGERRDFDPRFDYSPKAFDRVPWKSIALAVFLLFLGCLLLLLSFFIFIGHMEGDSSQGYALLVLGILTFLPGFYETRIAYYSWRGAEGYRFAAIPSY

SEQ ID NO: 84 - protein, 120 aa, Brassica napus
  misc_feature (1)..(120): Ceres CLONE ID no. 975428
MASRRNVRYAQLPGEEDDEDYANGGGRRDFDPRFEYTPKAFDRVPWKSIALALFLLFLGCLLLLLTVFIFTGHMEGDSSQGYALLVLGILTFLPGFYETRIAYYSWRGAEGYRFAAIPSY

SEQ ID NO: 85 - protein, 120 aa, Brassica napus
  misc_feature (1)..(120): Ceres CLONE ID no. 965227
MASRRSVRYAQLPGDDEDYADGGGGGRRDFDPRFDYTPKAFDRVPWKSIGLAVFLLVLGCLLLLLAVFIFTGHMEGDSSQGYALLVLGFLTFLPGFYETRIAYYSWRGAEGYRFAAIPSY

SEQ ID NO: 86 - protein, 116 aa, Glycine max
  misc_feature (1)..(116): Ceres CLONE ID no. 635196
MTTRRVRYSPLATDEDDYIGDRNRPFDPRFDYTPKALDKVPWKSIALALFLLFLGTGLLFLSYFIFTGHMGGERSQAYGLLALGFLSFLPGFYETRIAYYAWRGAKGYRFSAIPDY

SEQ ID NO: 87 - DNA, 348 bp, Populus balsamifera subsp. trichocarpa
  misc_feature (1)..(348): Ceres ANNOT ID no. 1506868
atggcatcaaggcgcaatgttcgttacagttctcttcctgatgatgacaatgatgataaatttggtgtacaatatgaccctcgatttgattatacacctggatcttttgatagagtcccatggaagtccattttccttgcaatttttctgctcttccttggatgtgtacttctctctctgtcattctttatcttcactggtcacatgggaggagagaagtcccaagcttatggtctcctagctctgggaatcattaccttcatgccgggcttttacgaaactcggatagcatattattcatggaggggtgctaagggatatcagtttgcttctatccctaaatattag

SEQ ID NO: 88 - protein, 115 aa, Populus balsamifera subsp. trichocarpa
  misc_feature (1)..(115): Ceres ANNOT ID no. 1506868
MASRRNVRYSSLPDDDNDDKFGVQYDPRFDYTPGSFDRVPWKSIFLAIFLLFLGCVLLSLSFFIFTGHMGGEKSQAYGLLALGIITFMPGFYETRIAYYSWRGAKGYQFASIPKY

SEQ ID NO: 89 - protein, 112 aa, Triticum aestivum
  misc_feature (1)..(112): Ceres CLONE ID no. 891349
MATRRNIPYSVLPTEDRDEDNVDRRFTYTPKSLRRIPWKSIALALFLLFLGSSLLFLSYFISTGHMEGDSSQVYGLLFLGILAFLPGFYETRVAYYSWRGAPGFTFAAIPDY

SEQ ID NO: 90 - protein, 119 aa, Triticum aestivum
  misc_feature (1)..(119): Ceres CLONE ID no. 1054465
MAARRNIPYSVLPTEDRDEDNVDRRFTYTPKSLRRIPWKSIALALFLLFLGSSLLFLSYFINTGHMEGDSSQVYGLLFLGILAFLPGFYETRVAYYSWRGAPGFTFAAIPDYXASIXTH

SEQ ID NO: 91 - protein, 117 aa, Zea mays
  misc_feature (1)..(117): Ceres CLONE ID no. 1602143
MAARRNVRYSPLPSEDGDDSNFIKEDVDLRYTYTPKSYRRIPWKSIALALFLLLLGTSLLFLSYFIFTGHMEGDSSQAYGLLFLGFLSFLPGFYETRVAYYSWRGAPGYTFASIPDY

SEQ ID NO: 92 - protein, 117 aa, Oryza sativa subsp. japonica
  misc_feature (1)..(117): Public GI no. 77548568
MASRRNVGGYAPLPTEDRDDSNLTDDVDLRFTYTPKSLRKIPWKSIALALFLLLLGCSLLFLSYFIFTGHMEGDNSQAYGLLFLGILAFLPGFYETRVAYYSSRGAPGYTFASIPDY

SEQ ID NO: 93 - protein, 118 aa, Oryza sativa subsp. japonica
  misc_feature (1)..(118): Public GI no. 77553579
MASRRNVRGYAPLPTEDRDDSNLTDDVDLRFTYTPKSLRKIPWKSIALALFLLLLGCSLLFLSYFIFTGHMEGDNSQAYGLLFLGILAFLPDSRQTSLLLYPFSEKDYIVLTLFCRFL

SEQ ID NO: 94 - DNA, 849 bp, Arabidopsis thaliana
  misc_feature (1)..(849): Ceres CLONE ID no. 8166
aaacaaaaaatctcttaacaacttctccaccttctccccaccaccaccactcaccgtcggagaaagaaaaaaaaacaaccaccatgaaagactgtgaaaaccacggccactctcgccggaaactaataaggcgaatattttggtcaataatcttcgtcctcttcatcatcttcctaacaatcctcctcatttgggcaattctccaaccttcaaagccacgcttcatcctccaagacgccaccgtctacgccttcaacgtctccggcaatccaccgaacctcctcacctccaacttccaaatcactctctcttcccggaaccctaacaacaaaatcggcatttactacgaccggcttgacgtctacgctacttaccggagccaacaaatcactttcccaacatcaatccctcccacgtatcagggacacaaagatgtcgacatctggtcgccgtttgtttacggaacctccgttcccatcgctccttttaacggcgttagtcttgacaccgataaagataacggtgtcgttttgttgatcattcgtgctgatggtagagtgaggtggaaggttgggacttttatcaccgggaagtatcatcttcatgtgaagtgtccagcatatataaacttcggtaacaaagctaacggagttatcgtcggagacaacgccgttaagtatacgtttaccactagttgtagtgttagtgtgtgaaacggcgacgaagaaaaataaggtaactaaacgtcttcgttaagtaatctgtcttagtgatgtgaattgtacattttgcttatacgaatagcaaatattacagattcgtgatgttgtaacgaattttaagaatttc

SEQ ID NO: 95 - protein, 209 aa, Arabidopsis thaliana
  misc_feature (1)..(209): Ceres CLONE ID no. 8166
MKDCENHGHSRRKLIRRIFWSIIFVLFIIFLTILLIWAILQPSKPRFILQDATVYAFNVSGNPPNLLTSNFQITLSSRNPNNKIGIYYDRLDVYATYRSQQITFPTSIPPTYQGHKDVDIWSPFVYGTSVPIAPFNGVSLDTDKDNGVVLLIIRADGRVRWKVGTFITGKYHLHVKCPAYINFGNKANGVIVGDNAVKYTFTTSCSVSV

SEQ ID NO: 96 - protein, 208 aa, Zea mays
  misc_feature (1)..(208): Ceres CLONE ID no. 1064651
MSKDCGNHGGGKEAAVRRICAAVIAFIIIVLITIFLVWVILRPTKPRFILQEATVFAFNLSQPNLLTTNFQITFASRNPNSKIGIYYDRLHVYATYRNQQITLRTAIPPTYQGHKEDNVWSPFVYGTAVPIAPYNSVALGDEQGHGLVQLMIRADGRVRWKVGTLITGKYHIHVRCPALINLGNKAAGVIVGDNAVKYTLVTKCSVNV

SEQ ID NO: 97 - protein, 209 aa, Brassica napus
  misc_feature (1)..(209): Ceres CLONE ID no. 970655
MSKDCGNHGGGKEAAVRRICAAVIAFIIIVLITIFLVWVILRPTKPRFILQDATVFAFNLSQPNLLTTNFQITFASRNPNSKIGIYYDRLHVYATYRNQQITLRTAIPPTYQGHKEDNVWSPFVYGTAVPIXPYNSVALGDEQGHGLVQLMIRADGRVRWKVGTLIXGKXHIHVRCPALINLGNKAAGVIVGDNAVKYTLVTKCSVNVX

SEQ ID NO: 98 - DNA, 630 bp, Populus balsamifera subsp. trichocarpa
  misc_feature (1)..(630): Ceres ANNOT ID no. 1475146
atgtcatccaaagactgcggcagccacggccacagacgccgaaaaatcttccgtcgaatcttcgccggaatcctaatcttcctcctcatagtcctcatcataattctcttagtttgggccatcctccgtccttcaaaaccaaaattcatcctccaagatgctacagtttacgccttcaacgtttcatctccaaatgttctcacttccaacttccaagtcaccttatcctctcgcaaccccaatgacaaagtcgggatctactatgataaactcgatgtttatgccacataccgtaaccaacaaataactctacgtacttccatacctacttcttaccaaggacacaaagaaattgatgtttggtccccttttatctacggcagcgccgtgcctgtatctccgtacaattcagttgcattaagtcaagaccaagctacaggaactgtgttgctgatgatcaagattgatggtcgtgtccgattcaaagttggaacctttatttctgcgaaatatcatttgaatgttcggtgtcctgcttatattcagtttggtagcagaaccagtggaatcattgtcggtgagaacgccattaagtatcagctggtaacgagatgcagtgtgagcctttga

SEQ ID NO: 99 - protein, 209 aa, Populus balsamifera subsp. trichocarpa
  misc_feature (1)..(209): Ceres ANNOT ID no. 1475146
MSSKDCGSHGHRRRKIFRRIFAGILIFLLIVLIIILLVWAILRPSKPKFILQDATVYAFNVSSPNVLTSNFQVTLSSRNPNDKVGIYYDKLDVYATYRNQQITLRTSIPTSYQGHKEIDVWSPFIYGSAVPVSPYNSVALSQDQATGTVLLMIKIDGRVRFKVGTFISAKYHLNVRCPAYIQFGSRTSGIIVGENAIKYQLVTRCSVSL

SEQ ID NO: 100 - protein, 209 aa, Glycine max
  misc_feature (1)..(209): Ceres CLONE ID no. 465057
MSVKECEHHKGKKRKIFRQVFWCLVVFLFIVLVTILLIWAILRPTKPTFTLQDVTVYAFNATVANFLTSNFQVTLISRNPNDRIGVYYDRLETFVTYRSQQVTYRTAIPPTYQGHKEINVWSPFVYGTNIPVAPFNFLGLSQDQSNGNVLVTIRAEGRVRWKVGTFISGRYHLYVRCPAFISFGPRSNGIVVGENAIKFQIIQRCSVSV

SEQ ID NO: 101 - protein, 208 aa, Glycine max
  misc_feature (1)..(208): Ceres CLONE ID no. 650444
MSVKECHHHKGKKHKLWRRIFWGIVIFAFIVLLTVLIIWAILKPSKPTFILQDVTVYGFNATIPNFLTSSFQVTLSSRNPNDKIGVYYDRLDTYVTYRNQVTYRTSIPPSYQGHKEEDVWSPFVFGTNVPVAPFNFVGLSQDQTNGNVLVLVKIDGKVRWKVGTFVSGHYNLYVRCPAFITFGPQSTGIALGKNAVKYQLVQRCTVGV

SEQ ID NO: 102 - protein, 210 aa, Glycine max
  misc_feature (1)..(210): Ceres CLONE ID no. 662698
MSVKECHHHHKGKKHKLWRRIFWGIVIFAFIVLLTVLIIWAILKPSKPTFILQDVTVYGFNATIPNFLTSSFQVTLSSRNPNDKIGIYYDRLNTYVTYRNQQVTYRTSIPPSYQGHKEEDVWSPFVYGTNVPVAPYNFVGLSQDQTNGNVLVLVKIDGKVRWKVGSFVSAHYNLNVRCPAFITFGPQSNGIALGNNAVKYQLVQRCTVGV

SEQ ID NO: 103 - protein, 210 aa, Oryza sativa subsp. japonica
  misc_feature (1)..(210): Public GI no. 62701864
MGKDCGNHGDDDIRQACRRLLTILFGLALIVAIIALIVYLVLRPTHPRFFLQDATLRQLDLSNSSTSGVLSTALQVTVASRNPNDRVGVYYDRLDVYASYKYQQITLAASLPPVYQGHGDVDVWSPVLSGPDVPFAPYLGDALAKDVAAEYLILQVKIDGRVRWKVGSWISGHYHLFVTCPAFFIASGGNGYPGANGLKFQTATYCRVEV

SEQ ID NO: 104 - protein, 213 aa, Triticum aestivum
  misc_feature (1)..(213): Ceres CLONE ID no. 632710
MSKDCGNHGEDDIRRTCRRFLAFLFFLALVVAVIALIVYLVLRPTHPRFYLQDASLRQLDVLTANASAAAGVLSTVLQVTVASRNPNDRVGVYYDRLDVYASYKYQQITLASALPPVYQGHGDVEVWSPVLSGPNVPFAPYLADALAKDVQAGYLILQVKIDGRVRWKVGSWISGHYHIFVTCPAFLIGTGGNGAPGANGLRFQTATYCHVEV

SEQ ID NO: 105 - protein, 212 aa, Oryza sativa subsp. japonica
  misc_feature (1)..(212): Public GI no. 77553726
MGKDCGKHGDDDFRQGCRRFITVLVVLAILVGIIALIVYLVLRPTHPRFYLQDATLRQLDLSNSSSTAGGVLSTTIQVTVASRNPNDRVGVYYDRLDVYASYKYQQITVAASLPPVYQGHGDVDVWSPVLAGPSVPFAPYLADAISQDCQAGYLILQVKIDGRVRWKVGSWISGHYHLFVTCPAFLVTAGGNGSPGASGFRFQTTTYCHVEV

SEQ ID NO: 106 - DNA, 1262 bp, Arabidopsis thaliana
  misc_feature (1)..(1262): Ceres CLONE ID no. 38311
aaagcaacaaaaacttaacccatttcttcttctttttttgtttctctctcacaaacacaacaaatatggaatacagctgtgtagacgacagtagtactacgtcagaatctctctccatctctactactccaaagccgacaacgacaacggagaagaaactctcttctccaccggcgacttcgatgcgtctctacagaatgggaagcggcggaagcagcgttgttttggattcagagaacggcgtcgagaccgagtcacgaaagcttccgtcgtcgaaatacaaaggcgttgtgcctcagcctaacggaagatggggagctcagatttacgagaagcatcagcgagtttggctcggtactttcaacgaggaagaagaagctgcgtcttcttacgacatcgccgtgaggaggttccgaggccgcgacgccgtcactaacttcaaatctcaagttgatggaaacgacgccgaatcggcttttcttgacgctcattctaaagctgagatcgtggatatgttgaggaaacacacttacgccgatgagtttgagcagagtagacggaagtttgttaacggcgacggaaaacgctctgggttggagacggcgacgtacggaaacgacgctgttttgagagcgcgtgaggttttgttcgagaagactgttacgccgagcgacgtcgggaagctgaaccgtttagtgataccgaaacaacacgcggagaagcattttccgttatcggcgatgacgacggcgatggggatgaatccgtctccgacgaaaggcgttttgattaacttggaagatagaacagggaaagtgtggcggttccgttacagttactggaacagcagtcaaagttacgtgttgaccaagggctggagccggttcgttaaagagaagaatcttcgagccggtgatgtggtttgtttcgagagatcaaccggaccagaccggcaattgtatatccactggaaagtccggtctagtccggttcagactgtggttaggctattcggagtcaacattttcaatgtgagtaacgagaaaccaaacgacgtcgcagtagagtgtgttggcaagaagagatctcgggaagatgatttgttttcgttagggtgttccaagaagcaggcgattatcaacatcttgtgacaaattctttttttttggttttttttcttcaatttgtttctcctttttcaatattttgtattgaaatgacaagttgtaaattaggacaatacaagaaaaaatgacaactag

SEQ ID NO: 107 - protein, 361 aa, Arabidopsis thaliana
  misc_feature (1)..(361): Ceres CLONE ID no. 38311
MEYSCVDDSSTTSESLSISTTPKPTTTTEKKLSSPPATSMRLYRMGSGGGSVVLDSENGVETESRKLPSSKYKGVVPQPNGRWGAQIYEKHQRVWLGTFNEEEEAASSYDIAVRRFRGRDAVTNFKSQVDGNDAESAFLDAHSKAEIVDMLRKHTYADEFEQSRRKFVNGDGKRSGLETATYGNDAVLRAREVLFEKTVTPSDVGKLNRLVIPKQHAEKHFPLSAMTTAMGMNPSPTKGVLINLEDRTGKVWRFRYSYWNSSQSYVLTKGWSRFVKEKNLRAGDVVCFERSTGPDRQLYIHWKVRSSPVQTVVRLFGVNIFNVSNEKPNDVAVECVGKKRSREDDLFSLGCSKKQAIINIL

SEQ ID NO: 108 - protein, 315 aa, Arabidopsis thaliana
  misc_feature (1)..(315): Ceres CLONE ID no. 19561
MGSGGSSVVLDPENGLETESRKLPSSKYKGVVPQPNGRWGAQIYEKHQRVWLGTFNEQEEAARSYDIAACRFRGRDAVVNFKNVLEDGDLAFLEAHSKAEIVDMLRKHTYADELEQNNKRQLFLSVDANGKRNGSSTTQNDKVLKTREVLFEKAVTPSDVGKLNRLVIPKQHAEKHFPLPSPSPAVTKGVLINFEDVNGKVWRFRYSYWNSSQSYVLTKGWSRFVKEKNLRAGDVVTFERSTGLERQLYIDWKVRSGPRENPVQVVVRLFGVDIFNVTTVKPNDVVAVCGGKRSRDVDDMFALRCSNKQAIINAL

SEQ ID NO: 109 - protein, 351 aa, Glycine max
  misc_feature (1)..(351): Public GI no. 72140114
MDGGCVTDETTTSSDSLSVPPPSRVGSVASAVVDPDGCCVSGEAESRKLPSSKYKGVVPQPNGRWGAQIYEKHQRVWLGTFNEEDEAARAYDIAALRFRGPDAVTNFKPPAASDDAESEFLNSHSKFEIVDMLRKHTYDDELQQSTRGGRRRLDADTASSGVFDAKAREQLFEKTVTPSDVGKLNRLVIPKQHAEKHFPLSGSGDESSPCVAGASAAKGMLLNFEDVGGKVWRFRYSYWNSSQSYVLTKGWSRFVKEKNLRAGDAVQFFKSTGPDRQLYIDCKARSGEVNNNAGGLFVPIGPVVEPVQMVRLFGVNLLKLPVPGSDGVGKRKEMELFAFECCKKLKVIGAL

SEQ ID NO: 110 - protein, 386 aa, Capsicum annuum
  misc_feature (1)..(386): Public GI no. 33320073
MEGTSSIDQESTTSDSLSIAPMTTTKPPESLCRMGSGTSSVIIDGENGVEAESRKLPSSKYKGVVPQPNGRWGAQIYEKHQRVWLGTFNEENEAARAYDVAAQRFRGRDAVTNFKPLLENQESDDDVEIAFLNSHSKAEIVDMLRKHTYIDELEQSKKLFGYTKDGTMAKNKDGLIDISSFFGGGGTIDKVNNKVREQLFEKAVTPSDVGKLNRLVIPKQHAEKHFPLQNGNNSKGVLLNFEDLNGKVWRFRYSYWNSSQSYVLTKGWSRFVKEKNLKAGDIVSFQRSTSGDKQLYIDFKARNMAPTNPVVTNQVQAQVQVPRVQMMRLFGVNICKIPATINNVVDNNNNNNNNMANCSGGKRMMEMELLTFESCRKKQRVIIDAL

SEQ ID NO: 111 - protein, 357 aa, Glycine max
  misc_feature (1)..(357): Ceres CLONE ID no. 597624
MSSEKASPSPPPPNRLCRVGSGASAVVDSDGGGGGGSTEVESRKLPSSKYKGVVPQPNGRWGSQIYEKHQRVWLGTFNEEDEAARAYDVAVQRFRGKDAVTNFKPLSGTDDDDGESEFLNSHSKSEIVDMLRKHTYNDELEQSKRSRGFVRRRGSAAGAGNGNSISGACVMKAREQLFQKAVTPSDVGKLNRLVIPKQHAEKHFPLQSAANGVSATAAAAKGVLLNFEDVGGKVWRFRYSYWNSSQSYVLTKGWSRFVKEKNLKAGDTVCFQRSTGPDRQLYIDWKTRNVVNEVALFGPVVEPIQMVRLFGVNILKLPGSDSIANNNNASGCCNGKRREMELFSLECSKKPKIIGAL

SEQ ID NO: 112 - protein, 365 aa, Oryza sativa subsp. japonica
  misc_feature (1)..(365): Public GI no. 34895690
MGVVSFSSTSSGASTATTESGGAVRMSPEPVVAVAAAAQQLPVVKGVDSADEVVTSRPAAAAAQQSSRYKGVVPQPNGRWGAQIYERHARVWLGTFPDEEAAARAYDVAALRYRGRDAATNFPGAAASAAELAFLAAHSKAEIVDMLRKHTYADELRQGLRRGRGMGARAQPTPSWAREPLFEKAVTPSDVGKLNRLVVPKQHAEKHFPLRRAASSDSASAAATGKGVLLNFEDGEGKVWRFRYSYWNSSQSYVLTKGWSRFVREKGLRAGDTIVFSRSAYGPDKLLFIDCKKNNAAAATTTCAGDERPTTSGAEPRVVRLFGVDIAGGDCRKRERAVEMGQEVFLLKRQCVVHQRTPALGALLL

SEQ ID NO: 113 - DNA, 1079 bp, Arabidopsis thaliana
  misc_feature (1)..(1079): Ceres CLONE ID no. 109289
accttctaaacattcacaccatctctttctctaacaatggagatttttcaaaaggccaaagccgttcgtctacggagccatcacgacaagtacctagtcgccgatgaagacgaagaatctgtaactcaagaacgtaacggctcagcaggtgcagccaaatggaccgttgagataatcccaggctcaacaaatctcatccgtctcaaaagcgcttatggcaaatacttaacagcgtctaacaaaccgtttcttcttggagccaccggaaaaaaagttctacagactaaacctagtcgtcttgactcgtctttagcttgggaaccgattagagattctgctttggttaagctcaagactcgttatggtaacttccttcgtggtaacggtggtttacctccctggagaaactctgttactcatgatattcctcatagatcggccactcaagaatgggtactttggcatatagatgtcgttgagatcctccctgcaaacgcaaactccgatcaccaccaccagcacctgcagctgcagcaacctccgtcgccgcttcatcattctgattctcttgatttcacacctggatctccttccagatctgaccgtttctttagacaagagtcaacggattcactagcggttggatccccaccgaaatcggaagggagagtgatatactatcatgtggcggatgatgatgatgatgttgaagatgattctgttgaggtaagttcgtttactttcaaaggaaatggtgttgaagagctgactatgagattgaaagaagagagcaatgtcgaagatgttatcgtttgtactcgtagtccgttgaacgggaagcttttcccattgcgtcttcaacttccacctaacaatgcagacatgactgttgtcttagtaccaaaatcatcaaaaatagcagaagagttcatcaagtgagatctttcttttgaagctgagctgtgaagtgaaggctttggctgcaattggtttgttgttactaaaaaggatttggattgacgtagagatactgcttgattaccaaacttatgatttataccattctttcccttttgtcc

SEQ ID NO: 114 - protein, 300 aa, Arabidopsis thaliana
  misc_feature (1)..(300): Ceres CLONE ID no. 109289
MEIFQKAKAVRLRSHHDKYLVADEDEESVTQERNGSAGAAKWTVEIIPGSTNLIRLKSAYGKYLTASNKPFLLGATGKKVLQTKPSRLDSSLAWEPIRDSALVKLKTRYGNFLRGNGGLPPWRNSVTHDIPHRSATQEWVLWHIDVVEILPANANSDHHHQHLQLQQPPSPLHHSDSLDFTPGSPSRSDRFFRQESTDSLAVGSPPKSEGRVIYYHVADDDDDVEDDSVEVSSFTFKGNGVEELTMRLKEESNVEDVIVCTRSPLNGKLFPLRLQLPPNNADMTVVLVPKSSKIAEEFIK

SEQ ID NO: 115 - protein, 295 aa, Glycine max
  misc_feature (1)..(295): Ceres CLONE ID no. 566154
MELFHRAKAVRLRSHHDKYLLADEDEESVTQDRNGSSRNAKWTVELIPEFDNIIRLKSCYGKYLTASNQPLLLGVTGRKVVQSLPRRLDSSVEWEPVRDGAQVKLKTRYGNFLRANGGVPPWRNSVTHDIPHRTATQDWILWDVDVLEIHVVSPAPPPIPHSDSLDFESNTPSAVNIKSTTFSRQESTDSNVGSPPKKMEGRTIYYHVAEDNGDVDDENVQGYSLNFKGNGVEQLARKFEEETGLEGVIVCTRSPLNGKLYPLRLQLPPNNVTMQVVLVLPSSKVAREFEEQGIL

SEQ ID NO: 116 - protein, 297 aa, Glycine max
  misc_feature (1)..(297): Ceres CLONE ID no. 541790
MDFFHRAKAVRLRSHHDKYLLAEDDEDSVTQDRKGSSKSARWTVEYVAEYDNIIRLKSCYGKYLTASNHRFLLGMTGHQVLQTLPSRLDSSVEWEPVKEGGRVKLKTRYGNFLRANGGLPPWRNSVTHDIPHRTATQDWILWDVDVVEIYVNSPASHDRPSAPPLPVEDSVPILTSAQVPPPPTVSASFSRQQSNDLSPKVEGRTIYYHIAEDNGEVLDEGVQGYSLIFKGNGVEELTRKFEEETGLEGIIVCNRSPLNGKLYPLRLQLPPNTVTMRVVLVLPLSNLARDFEEQGLL

SEQ ID NO: 117 - protein, 284 aa, Zea mays
  misc_feature (1)..(284): Ceres CLONE ID no. 218121
MELFPHGGFVRLRSRARDKYVHADVDASGVSLRPLGAAPSVNAVWKPERWPSEGDFLILQIVAYGRYLAVSDKDAPPGHRGVRAVQRDYNSPRAEAPFLWTAFRVDAHQNYVRLHNHQRWLRANGRHRYWNNVVTIDTRNASLTTMMQWRVEEIPVSPEPLPLPPPPQQPTIHVSRGLFKRRAKVHPSGRMIQHVRMDDDGFILDNWPDFNFDDYSVSNLRAEVALRQDDENITLCXRAGKHALLIPLITDLPHNTDPLDIIVMPAGSPGLDALEYPQFDAPEP

SEQ ID NO: 118 - DNA, 1241 bp, Arabidopsis thaliana
  misc_feature (1)..(1241): Ceres CLONE ID no. 19342
aagaatctagtgaatttccgattccgattttgagctaaaggaagcgagagaagaaaagatgtcgggtatgggtgacggctacgttggtacggctcaagatgcggtgaggatacggcggcttcagaagcaaagagaggccgaacgcaaaaaaatacaagagctcaagagcaagtctgcctccggcaatgaccaatctggtcttctccaattcggcaccagctcgtgcgagattcttgacactgctttcaagaaggaaacagtgggtttagttacaagagaggagtatgtggagaagagggttaatattcgtaataagttcgaggaagaagagaaggagaaactgcagaagctacagcaagaggaagaggagttgcagctagagaagcgtaacaagaagagaaagattaagggatcttcccgcttatcttttgctgaagattttgagaatggcagtgatgaagatgatggagaaaacaagagttctgggacagggaacttacgctgcggaaaacttggcaaggacccctcagtggaaacgaatttcctgcctgacagtgagagggaggcggaggaacaagctgaacgtgaaaggctgaagaagcagtggcttcgtgaacaagagcagattaaaaacgagcctcttgagattacttacagttacagggatgggactggccatagacgagttattcaggtccgcaagggtgacccaattgggaattttctgcgggctgttcagcagcagcttgcccctgacttccgggagattcggacggcatcagtggagaatttactatatgtaaaggaagatcttattattccacatcaacacagtttctatgagctgatcattaacaaagcgagggggaagagtgggccgctgtttcactttgatgtgcatgaagacgtgagaacaatagcagatgcaacgatagagaaagacgagtcgcatgcgggtaaagttgtggagaggcattggtacgagaagaacaagcatatattccctgcttccagatgggagatttacgacccgacaaagaaatgggaacggtacacagtccatggggattaattctggaccagaacatgcttttgatggatcgtttgttgacatgattgatcagatttggtgtaatagtagtggaatgttcatggcatttttaatgctcattcttcttccctcaatgtaccattacactcgttttttaatttttcttaaaataatggaactgtttgattcc

SEQ ID NO: 119 - protein, 337 aa, Arabidopsis thaliana
  misc_feature (1)..(337): Ceres CLONE ID no. 19342
MSGMGDGYVGTAQDAVRIRRLQKQREAERKKIQELKSKSASGNDQSGLLQFGTSSCEILDTAFKKETVGLVTREEYVEKRVNIRNKFEEEEKEKLQKLQQEEEELQLEKRNKKRKIKGSSRLSFAEDFENGSDEDDGENKSSGTGNLRCGKLGKDPSVETNFLPDSEREAEEQAERERLKKQWLREQEQIKNEPLEITYSYRDGTGHRRVIQVRKGDPIGNFLRAVQQQLAPDFREIRTASVENLLYVKEDLIIPHQHSFYELIINKARGKSGPLFHFDVHEDVRTIADATIEKDESHAGKVVERHWYEKNKHIFPASRWEIYDPTKKWERYTVHGD

SEQ ID NO: 120 - DNA, 1011 bp, Populus balsamifera subsp. trichocarpa
  misc_feature (1)..(1011): Ceres ANNOT ID no. 1450498
atgtcgggcatgggagacgggtacgtgggcactgcccaagacgccgtgaggatccggcggctacagaagcaaagagaagctgaacgccttaaaatccaagagctcaaatctaagtcagcctccgctaaggaccagcccggtctcctccaattcgggtcaagtacatccgagattctcgagactgcatttaagaaggaaactgtaggtctggttacaagagaacagtatgtagagaagagggttaatattcagaccaaatttgaagaagaagagaaggagaagttagaaaagctacggaaagaggaggaggagcttcagttgcagaagcggaaaaagaggaagattaaggggaattctaagttatcgtttgctgatgattttgagaatggaagtgatgaagaggatggcgaaaataaaagcttagagccaaagagattagtgaacggtaaatttggaaaagatcccacagtggaaacaagctttttgcctgacagtgagcgagaggcagaggagcaagctgagcgtgaaaggctgcggaaacagtggcttcgtgaacaggaacagattcgaaatgagccccttgaaatcacttacagctactgggatggagcaggccatagaagagtcatccaggttcgtaaaggtgataccataggagagtttcttcgggcagttcagcaacaacttgcgccagagttcagagaaattagaacaacttctgtggagaatttgctttatgtgaaagaggatcttatcattccccatcaacacagtttctatgacctgatcattaacaaggctaggggaaaaagtggtccgcttttccactttgatgtgcacgaggatgtgcgaacaattgctgatgcaactatagagaaggatgagtcccatgccggtaaagttgttgagaggcactggtatgaaaagaataaacacatctttcctgcttcaagatgggagatatatgacccgacacagaagtgggagcgttacaccatccatggggattga

SEQ ID NO: 121 - protein, 333 aa, Populus balsamifera subsp. trichocarpa
  misc_feature (1)..(333): Ceres ANNOT ID no. 1450498
MGDGYVGTAQDAVRIRRLQKQREAERLKIQELKSKSASAKDQPGLLQFGSSTSEILETAFKKETVGLVTREQYVEKRVNIQTKFEEEEKEKLEKLRKEEEELQLQKRKKRKIKGNSKLSFADDFENGSDEEDGENKSLEPKRLVNGKFGKDPTVETSFLPDSEREAEEQAERERLRKQWLREQEQIRNEPLEITYSYWDGAGHRRVIQVRKGDTIGEFLRAVQQQLAPEFREIRTTSVENLLYVKEDLIIPHQHSFYDLIINKARGKSGPLFHFDVHEDVRTIADATIEKDESHAGKVVERHWYEKNKHIFPASRWEIYDPTQKWERYTIHGD

SEQ ID NO: 122 - DNA, 1011 bp, Populus balsamifera subsp. trichocarpa
  misc_feature (1)..(1011): Ceres ANNOT ID no. 1460687
atgtcgggcatgggagacgggtacgtgggcacggcccaagacggcgtgaggatacggcggttacagaagcagagagaagctgaacgccttaaaatccaagagctcaaaaccaagtctgcctccgataacggccagcctggtctcctccaattcgggtcaagtacctccgagattcttgagactgcatttaagaaggaaactgtgggtttggttacaagagaacagtacgtggagaagagggttaatattcagaccaagtttgaagaagaagagaaggagaaactagaaaagctacggaaagaggaggaggatcttcagttgcagaagcgtaaaaagaggaagattaaggggagttctaagttgtcttttgcggatgattttgagaatggaagtgacgaagaggatgttgaaaacaaaacctcagagccaaataatttagtgcgggggaaatttggaaaagatcccactgtggaaacaagctttttgcctgacagtgagcgagaggcagaggagcaggctgagcgtgaaaggctgcggaaacagtggcttcgtgaacaggaacagattcaaaatgagccccttgaaatcacttacagctactgggatggagcaggccatagaagagtgatccaggttcgtaaaggtgatgccataggagagtttcttcggggagttcagcaacaacttgcgccagagttcagagaaattaggactacttctgtggagaatttgctttatgtgaaagaggatcttatcattcctcatcaacacagtttctatgagttgatcgttaacaaagctaggggaaaaagtggaccgctcttccactttgacgtgcacgaggatgtgcgaacaattgctgatgcaaccatagagaaggatgagtcccatgctggtaaagttgttgagaggcactggtatgaaaagaacaaacacatctttcctgcttcgagatgggagatatatgatccgacaaagaagtgggagcgttacaccatccacggggattga

SEQ ID NO: 123 - protein, 333 aa, Populus balsamifera subsp. trichocarpa
  misc_feature (1)..(333): Ceres ANNOT ID no. 1460687
MGDGYVGTAQDGVRIRRLQKQREAERLKIQELKTKSASDNGQPGLLQFGSSTSEILETAFKKETVGLVTREQYVEKRVNIQTKFEEEEKEKLEKLRKEEEDLQLQKRKKRKIKGSSKLSFADDFENGSDEEDVENKTSEPNNLVRGKFGKDPTVETSFLPDSEREAEEQAERERLRKQWLREQEQIQNEPLEITYSYWDGAGHRRVIQVRKGDAIGEFLRGVQQQLAPEFREIRTTSVENLLYVKEDLIIPHQHSFYELIVNKARGKSGPLFHFDVHEDVRTIADATIEKDESHAGKVVERHWYEKNKHIFPASRWEIYDPTKKWERYTIHGD

SEQ ID NO: 124 - protein, 338 aa, Glycine max
  misc_feature (1)..(338): Ceres CLONE ID no. 1043576
MSGMGDGYVGTAQDAVRIRRLEKQREAERRKIQELKTKSASAKDQPGLLQFGSSTSEILETAFKKETVGLVTREQYVEKRVNIQSKIEEEEKEKLQKQQQEEEELQLQKRKKRKIRGNSRLSFAEDIDNEPQEDEPHHSKDNLEANRLRCGKLGKDPTVETSFLPDSEREAEEQAERERLRKQWLREQEQIRNEPLEITYSYWDGTGHRRVIQVRKGDSIGEFLRAVQQQLAPEFREIRTTSVENLLYVKEDLIIPHQHSFYELIVNKARGKSGPLFHFDVHEDVRTIADATIEKDESHAGKVVERHWYEKNKHIFPASRWEIYDPTKKWERYTIHGD

SEQ ID NO: 125 - protein, 333 aa, Oryza sativa subsp. japonica
  misc_feature (1)..(333): Public GI no. 50726581
MSGFGDGYVGTAQDAVKIRRLEKQREAERRKIEELKNKSSDGQPGLLQFGSSTSEILETAFKKETVGLVTREQYVEKRVNIRTKIEEEEKEKLQKLQQEEEELQMQKRKKRRVRGDPRLSFCDEIENGSDEDEFENQEPQKKHGPVKLGKDPTVETSFLPDREREAEEQAERERLKKQWSREQELIKNEPLTITYSYWDGTGHRRVIQVRKGDSIGEFLRAVQQQLAPEFREVRTTSVENLLYVKEDLIIPHQHSFYELIINKARGKSGPLFHFDVHEDVRTIADATKEKDESHAGKVVERHWYEKNKHIFPASRWEIYDPTKKWERYTIHGD

SEQ ID NO: 126 - DNA, 619 bp, Arabidopsis thaliana
  misc_feature (1)..(619): Ceres CLONE ID no. 21006
atactcaaaacaaaacaaaacatacatcaaaacgctaaagtttaaacccctagccatcatcagatcttcagacttctgaggatcatggacaaagtgatgagaatgtcttcagagaaaggagtggtgatcttcacgaagagctcatgttgtctctgctacgccgttcagatcctgttccgtgaccttagggttcaaccaaccatccacgagatcgacaacgacccggactgccgtgagatcgagaaggctcttctccggctcggctgttccacggcggttccagctgtctttgtcggaggcaagcttgttggctccaccaatgaagtcatgtcccttcaccttagtggctctcttgtcccattgatcaaaccctatcagtccatcctttactagcaaaattaaaccaactcaatatataatatctaattattagctagtgagaataaacacagttacagctagagtgtgagctagctagatattcagtgaggacttcgtctgaattaatgtttatcgtttgtatgttctattgtttagcttctctcgtgtttcagtttagttaataaactggtgtatgttgatgtatgactctctgtttatgctaatgaaaatagtattg

SEQ ID NO: 127 - protein, 102 aa, Arabidopsis thaliana
  misc_feature (1)..(102): Ceres CLONE ID no. 21006
MDKVMRMSSEKGVVIFTKSSCCLCYAVQILFRDLRVQPTIHEIDNDPDCREIEKALLRLGCSTAVPAVFVGGKLVGSTNEVMSLHLSGSLVPLIKPYQSILY

SEQ ID NO: 128 - protein, 101 aa, Brassica napus
  misc_feature (1)..(101): Ceres CLONE ID no. 1079973
MRMSSEKGVVIFTKSSCCLCYTVQILFRDLRVQPTIHEIDNDPDCREIEKALLRIGCSTAVPAVFVGGKLVGSTNEVMSLHLSGSLVPLIKPYQSLLYQAN

SEQ ID NO: 129 - protein, 102 aa, Arabidopsis thaliana
  misc_feature (1)..(102): Public GI no. 7573425
MDKVMRMSSEKGVVIFTKSSCCLCYAVQILFRDLRVQPTIHEIDNDPDCREIEKALVRLGCANAVPAVFVSGKLVGSTNDVMSLHLSGSLVPLIKPYQSFHN

SEQ ID NO: 130 - protein, 98 aa, Brassica napus
  misc_feature (1)..(98): Ceres CLONE ID no. 953083
MRMSSEKGVVIFTKDSCCLCYAVQILFRDLRVQPTIHEIDNDPDCREIEKALVRLGCTNAVPAVFVSGKLVGSTNEVMSLHLSGSLVPLIKPYQSFHN

SEQ ID NO: 131 - protein, 98 aa, Triticum aestivum; identical to SEQ ID NO: 130
  misc_feature (1)..(98): Ceres CLONE ID no. 1030898

SEQ ID NO: 132 - protein, 98 aa, Brassica napus
  misc_feature (1)..(98): Ceres CLONE ID no. 940212
MRMSSGKGVVIFTKNSCCLCYAVQILFRDLRVQPTIHEIDNDPDCLEIEKALVRLGCPNAVPAVFVSGKLVGSTNEVMSLHLSGSLVPLIKPYQLFHN

SEQ ID NO: 133 - protein, 98 aa, Brassica napus
  misc_feature (1)..(98): Ceres CLONE ID no. 1070065
MRMSSEKGVVIFTKNSCCLCYAVQVLFRDLRVQPKIHEIDNDPDCREIEKALVRLGCANAVPAVFVSGKLVGSTNDVMSLHLSGSLVPLIKPYQAFHN

SEQ ID NO: 134 - protein, 99 aa, Arabidopsis thaliana
  misc_feature (1)..(99): Ceres CLONE ID no. 125679
MDKVMRMSSEKGVVIFTKSSCCLSYAVQVLFQDLGVNPKIHEIDKDPECREIEKALMRLGCSKPVPAVFIGGKLVGSTNEVMSMHLSSSLVPLVKPYLC

SEQ ID NO: 135 - protein, 99 aa, Arabidopsis thaliana; identical to SEQ ID NO: 134
  misc_feature (1)..(99): Public GI no. 21537263

SEQ ID NO: 136 - protein, 102 aa, Arabidopsis thaliana
  misc_feature (1)..(102): Public GI no. 24111317
MDKVVRMSSEKGVVIFSKSSCCMSYAVQVLFQDLGVHPTVHEIDKDPECREIEKALMRLGCSTPVPAIFVGGKLIGSTNEVMSLHLSGSLVPLVKPFQANLC

SEQ ID NO: 137 - protein, 96 aa, Arabidopsis thaliana
  misc_feature (1)..(96): Ceres CLONE ID no. 39560
MSSEKGVVIFSKSSCCMSYAVQVLFQDLGVHPTVHEIDKDPECREIEKALMRLGCSTPVPAIFVGGKLIGSTNEVMSLHLSGSLVPLVKPFQANLC

SEQ ID NO: 138 - protein, 101 aa, Brassica napus
  misc_feature (1)..(101): Ceres CLONE ID no. 871147
MSSDKGVVIFSKSSCCMSYAVQVLFQDLGVHPTVHEIDKDPDCREIEKALMRLGCSTPVPAIFVDGKLVGSTNEVMSMHLSGSLVXLVKPFQANPMLKCVL

SEQ ID NO: 139 - protein, 101 aa, Glycine max
  misc_feature (1)..(101): Ceres CLONE ID no. 510704
MDKVTRLATEKGVVVFTKSSCCLCYAVNILFQELGVIPVVHEIDKDPEGKEIEKAITRLGCPTPVPAVFIGGKLVGSTNEVMSLHLSGSLTQLLKPYRALS

SEQ ID NO: 140 - DNA, 306 bp, Populus balsamifera subsp. trichocarpa
  misc_feature (1)..(306): Ceres ANNOT ID no. 1525141
atggataaggtgttgagattggcctctgagcagggggtagtgatattcatcaagagcacatgttgcttgtgttatgcagtcaaaatcctgttccaagaaattggggtggaccctctggttcatgagattgaccaagaccctgaaggcagggaaatggaaaaggctctcacaaggatggggtgtagcgcgcctgtaccggctgtattcgttggtggaaagctgctgggatccaccaatgaagtcatgtccctccacctcagtggctcactcaatcaaatgctcaaaccctaccagtctcaaacttaa

SEQ ID NO: 141 - protein, 101 aa, Populus balsamifera subsp. trichocarpa
  misc_feature (1)..(101): Ceres ANNOT ID no. 1525141
MDKVLRLASEQGVVIFIKSTCCLCYAVKILFQEIGVDPLVHEIDQDPEGREMEKALTRMGCSAPVPAVFVGGKLLGSTNEVMSLHLSGSLNQMLKPYQSQT

SEQ ID NO: 142 - DNA, 300 bp, Populus balsamifera subsp. trichocarpa
  misc_feature (1)..(300): Ceres ANNOT ID no. 1472813
atggataaggtgatgggattggcctctgagaagggggtagtgatattcagcaagagctcatgttgcttgtgttatgcagtcaaaattcttttccaagaaattggggtggaccctctggtttatgagattgaccaagaccctgaaggcagggaaatggaaaaggctctcacaaggttggggtgtaacgcgcctgtaccggccgtttttatcggtggaaagctgatgggttccacgaatgaagtcatgtccctccacctaagtggctcactcattccaatgctcaaaccctaccagaactaa

SEQ ID NO: 143 - protein, 95 aa, Populus balsamifera subsp. trichocarpa
  misc_feature (1)..(95): Ceres ANNOT ID no. 1472813
MGLASEKGVVIFSKSSCCLCYAVKILFQEIGVDPLVYEIDQDPEGREMEKALTRLGCNAPVPAVFIGGKLMGSTNEVMSLHLSGSLIPMLKPYQN

SEQ ID NO: 144 - protein, 105 aa, Plantago major
  misc_feature (1)..(105): Public GI no. 53748489
MDKVMKIKSENGVVIFSKSSCCLSYAVQMLFQELRANAFIYEIDHSPDGKEVEKALMRLGSSGPIPAVFIGGKLVGSTNEVMSLHLSGSLIPLLKPTQANLSYKS

SEQ ID NO: 145 - protein, 103 aa, Oryza sativa
  misc_feature (1)..(103): Public GI no. 58737210
MDRVNRLAAQRAVVIFSMSSCCMCHTVTRLFCELGVNPTVVELDEDPRGKEMEKALARLLGRSPAVPAVFIGGRLVGSTDKVMSLHLSGNLVPLLRNAGALWV

SEQ ID NO: 146 - protein, 103 aa, Oryza sativa subsp. japonica
  misc_feature (1)..(103): Public GI no. 77556540
MDRVMKLASERAVVIFTLSSCCMCHTVTRLFCDLGVNALVHELDQDPRGKEMERALLKLLGRGPPVPVVFIGGKLVGGTNKIMSLHLGGELIPMLKNAGALWL

SEQ ID NO: 147 - DNA, 954 bp, Arabidopsis thaliana
  misc_feature (1)..(954): Ceres CLONE ID no. 2296
aacgaaccaaacctctctctgctcagcttttccgaccaaaagatccaagcgcaagatcaactcaaaatcaatggattatctcggaatcgacatgagctgcgcgatcggatctctccggaacggtgacttcccggagaaagattgtcttcttcctctaatttcaaagcttctcggttattgcctcgttgctgcttcaatcaccgtcaagctccctcagataatgaaaatcgttcaacataagagtgtacgaggcttaagtgttgtggcatttgagcttgaagttgttggttacacaatttcacttgcttattgtttgcataaaggtcttcccttttcggctttcggcgaaatggcttttcttttgatccaagctttaatcttggtggcttgtatctattattattcacaaccagtacctgtcacaacttggattagaccacttctatattgcgctgtggcgccaactgtgctttctggacagattaatccgacgcttttcgaagctctttatgcttcacaacatgcgatatttctctttgcaagactccctcagatatggaagaactttaaaaacaaaagcactggagaacttagtttcttaactttcttcatgaactttgccgggtcaatagtgagagttttcaccagcttacaggaaaaagctccaattagcattcttacgggtttcgctcttggagtcgtcaccaacggaagcatcctgacgcagattctcttgtactcgaagcctgctgctgcaaaggagaagaaagccaattgataggtagaaagatatcctaaatcaaatactggtgttgaggctccaaaaagagattagtcaatgttgcattcacacctttttctcttttaatatgttcaaggatgttcagactttagccggctactggcttcgacttgattctgatagtgaagcggctgttttaagtctgattgatc

SEQ ID NO: 148 - protein, 235 aa, Arabidopsis thaliana
  misc_feature (1)..(235): Ceres CLONE ID no. 2296
MDYLGIDMSCAIGSLRNGDFPEKDCLLPLISKLLGYCLVAASITVKLPQIMKIVQHKSVRGLSVVAFELEVVGYTISLAYCLHKGLPFSAFGEMAFLLIQALILVACIYYYSQPVPVTTWIRPLLYCAVAPTVLSGQINPTLFEALYASQHAIFLFARLPQIWKNFKNKSTGELSFLTFFMNFAGSIVRVFTSLQEKAPISILTGFALGVVTNGSILTQILLYSKPAAAKEKKAN

SEQ ID NO: 149 - protein, 185 aa, Glycine max
  misc_feature (1)..(185): Ceres CLONE ID no. 525163
MKILKHQSVRGLSMISFELEVIGYTIALAYCLHKGLPFSAYGELLFLLIQALVLVAIIYYYSRPLHAITWIRALLYCAVAPTILAGQIDPLLFEALYASQHAIFLFARIPQIWQNFSNKSTGELSFITSFMNFGGSMVRVFTTIQESAPKSVLLGYAIGVATNFTILSQIIAYQKPQVGKEKKTK

SEQ ID NO: 150 - protein, 244 aa, Oryza sativa subsp. japonica
  misc_feature (1)..(244): Public GI no. 50937115
MVAAAGTMELEILGINFGCVLAALADAKIPEKDCLLPLASKLLGYAIVAASTTVKLPQILKILKHGSVRGLSVASFELEVVGYTIALAYCIHKGLPFSAYGELAFLLIQAIILVAIIYYYSPPMGTKTWMKALLYCGLAPTVLGGKIDPALFEVLYASQHAIFFFARLPQIWKNFMNKGTGELSFLTCFMNFAGSIVRVFTSIQEKTPLSVILGSAIGIVMNGTLLGQIVLYQKPAPKKEKKRD

SEQ ID NO: 151 - protein, 237 aa, Zea mays
  misc_feature (1)..(237): Ceres CLONE ID no. 242812
MELEILGMNFGCVLAALSDAKIPDKDCLLPLISKLLGYAIVAASTTVKLPQILKILKHGSVRGLSVASFELEVVGYTIALAYCIHKGLPFSAYGELAFLLIQAIILVAIIYYYSPPMGTKTWMKALLYCGMAPTVLAGKIDPALFEVLYASQHAIFFFARVPQIWKNFTNKGTGELSFLTCFMNFAGSIVRVFTSIQEKTPLSVIMGSAIGIVMNGTLLGQIVLYQKPAPKKQKKED

SEQ ID NO: 152 - protein, 242 aa, Zea mays
  misc_feature (1)..(242): Ceres CLONE ID no. 243125
MSEGNRWGSGMKLDVMGMTIGCFISCPLHTNNLLPFISKILGYSIIAASTVGKLPQILKILKHGSVRGLSVASFELEVVGYTIALAYCIHKGLPFSAYGELAFLLIQAIILVAIIYYYSPPMGTKTWMKALLYCGMAPTVLAGKIDPALFEVLYASQHAIFFFARVPQIWKNFTNKGTGELSFLTCFMNFAGSIVRVFTSIQEKTPLSVIMGSAIGIVMNGTLLGQIVLYQKPAPKKQKKED

SEQ ID NO: 153 - protein, 246 aa, Triticum aestivum
  misc_feature (1)..(246): Ceres CLONE ID no. 687022
MWRLANLDTMELEILGMNFGCVLSALSDAKIPEKECLLPLVSKLLGYCIVAASTTVKLPQILKILKHGSVRGLSVASFELELIGYTIALAYCIHKGLPFSAYGELAFLLIQAIILIGIIYYYSPPMGSKTWMKALLYCGLAPTVLAGKIDPGLFEILYASQHAIFFCARVPQIWKNFTNKSTGELSFLTSFMNFAGSLVRVFTSIQEKTPLSVLMGSVIGIVTNGTILGQIAMYQKPVPKKGKKEE

SEQ ID NO: 154 - DNA, 503 bp, Arabidopsis thaliana
  misc_feature (1)..(503): Ceres CLONE ID no. 33038
attttatgcttaaagacttgttccaagccgtatcctatcaaaacactgcaaatctctctctctttcaagccttgtcggtggttgaatcgaaagctatgtctcagaccgttgttctcagagtggccatgacatgtgagggatgtgttggagctgtgaaaagagttcttgggaaaatggaaggcgtggagtcatttgacgttgatataaaggaacagaaagtgacggtgaaaagcaacgtgcagccagacgcggttttacagaccgtaacgaaaaccggaaagaaaacggctttttgggaaactgagggtgaaactgctaaggcttaaggagaagcagagcattttatgtgattctctgttttaataaagatctatgtggaatcgttttatctgtttcttgtgacttgtggctgagtgtattggaactatacatgatacaacacacttttgagtgaataaatatgcactcgggcttaatcgggtctgataaagcacgagcctatg

SEQ ID NO: 155 - protein, 106 aa, Arabidopsis thaliana
  misc_feature (1)..(106): Ceres CLONE ID no. 33038
MLKDLFQAVSYQNTANLSLFQALSVVESKAMSQTVVLRVAMTCEGCVGAVKRVLGKMEGVESFDVDIKEQKVTVKSNVQPDAVLQTVTKTGKKTAFWETEGETAKA

SEQ ID NO: 156 - protein, 106 aa, Arabidopsis thaliana
  misc_feature (1)..(106): Public GI no. 18655401
MLKDLFQAVSYQNTASLSLFQALSVVESKAMSQTVVLRVAMTCEGCVGAVKRVLGKMEGVESFDVDIKEQKVTVKGNVQPDAVLQTVTKTGKKTAFWEAEGETAKA

SEQ ID NO: 157 - protein, 77 aa, Brassica napus
  misc_feature (1)..(77): Ceres CLONE ID no. 1064435
MSQTVVLKVAMPCEGCVGAVKRVLGKMQGVESFDVDLKEQKVTVKGNVEPDAVLQTVSKTGKKTSFWGADEAETAKA

SEQ ID NO: 158 - protein, 79 aa, Triticum aestivum
  misc_feature (1)..(79): Ceres CLONE ID no. 622673
MSQTVELRVGMSCEGCVGAVKRVLGKMEGVESFDVDIKEQKVTVKGNVTPDAVLQTVSKTGKKTAFWEAEPSAASAVSS

SEQ ID NO: 159 - DNA, 336 bp, Populus balsamifera subsp. trichocarpa
  misc_feature (1)..(336): Ceres ANNOT ID no. 1465436
atggaggtggtaattggaatttggtactgcaagaggggatgggtgtactatcaatatgtgtgcagcaataccataatcatgtctcagactgttgtcctcaaggttggtatgtcatgcgaaggctgtgttggggctgtgaaaagggttttgggaaaaatggaaggtgtggaatcatatgacattgatttgaaggagcaaaaagtcacagtgaaaggaaatgtgcagccagatgctgttcttcagaccgtctctaagaccgggaagaagactgccttctgggaagcagaggcaccagctgaacccgcaaagcctgcagaaaccgtggctgctgcataa

SEQ ID NO: 160 - protein, 111 aa, Populus balsamifera subsp. trichocarpa
  misc_feature (1)..(111): Ceres ANNOT ID no. 1465436
MEVVIGIWYCKRGWVYYQYVCSNTIIMSQTVVLKVGMSCEGCVGAVKRVLGKMEGVESYDIDLKEQKVTVKGNVQPDAVLQTVSKTGKKTAFWEAEAPAEPAKPAETVAAA

SEQ ID NO: 161 - protein, 85 aa, Populus alba x Populus glandulosa
  misc_feature (1)..(85): Public GI no. 47176684
MSQTVVLKVGMSCEGCVGAVKRVLGKMEGVESYDIDLKEQKVTVKGNVQPDAVLQTVSKTGKKTAFWEAEAPAEPAKPAETVAAA

SEQ ID NO: 162 - protein, 81 aa, Lycopersicon esculentum
  misc_feature (1)..(81): Public GI no. 30039180
MSQTVVLKVGMSCEGCVGAVKRVLGKMEGVETFDIDLKEQKVTVKGNVQPDAVLKTVSKTGKPTSFWEAGESAQTEAVSTA

SEQ ID NO: 163 - protein, 71 aa, Glycine max
  misc_feature (1)..(71): Ceres CLONE ID no. 625242
MSCEGCVGAVKRVLGKLDGVESYDIDLKEQKVVVKGNVQPDTVLATVSKTGKKTTFWEGEAAASETSTATA

SEQ ID NO: 164 - protein, 84 aa, Brassica napus
  misc_feature (1)..(84): Ceres CLONE ID no. 944316
MAQTVVLKVGMSCEGCVGAVKRVLGKMEGVESYDVDIMEQKVTVKGNVTPDAVLQTVSKTGKKTSFWEAEAVTSESATPAGATA

SEQ ID NO: 165 - protein, 81 aa, Oryza sativa subsp. japonica
  misc_feature (1)..(81): Public GI no. 50942155
MAETVVLRVGMSCEGCVGAVKRVLGKMQGVESFDVDIKEQKVTVKGNVTPDAVLQTVSKTGKKTSFWDAEPAPVEATAASS

SEQ ID NO: 166 - DNA, 815 bp, Arabidopsis thaliana
  misc_feature (1)..(815): Ceres CLONE ID no. 5821
acaattcatatcctgtctgatgaacagtagctgttagactctctctctttctctcttctctgttcttcttcatcttctctagccgaatccatccttcagatactactctgtaatttcactacaatcatctcagccaaatgggttcggaagaagagaaggtcgttgttcccagaaactttagattattggaagagctagaaagaggtgagaaaggaattggagatgggacagtgagttatggaatggatgatgctgatgatattttgatgcaatcttggactggtaccattcttggtcctcataatactgcgtatgaagggaaaatctttcagctgaagctattctgtggaaaggattatccggaaagtccacctactgtgaggtttcagagtcgtataaacatggcttgtgtcaatcctgagaatggagtggttgatccgagtcacttccctatgctttccaactggagaagagaatacacaatggatgatctactgattcagcttaaaaaagaaatgatgtcatcgcagaaccgcaagttagctcaacccttggaaggtaatgaggaaggcaggacagacccaaagggactagtggtgaaatgttgcgtgatgtgaaaagaggtaatgaggaatttataagaatttaagagacaaaacaatgtatataaacagcatttgaagggctttgagattggcctaagaaccctttcttctacaggccttcttaatcctaccatttgattatgtattatgtaatatttgagaatatattcaccaaaaacaggtgcccttaataaacctccactcttcttc

SEQ ID NO: 167 - protein, 159 aa, Arabidopsis thaliana
  misc_feature (1)..(159): Ceres CLONE ID no. 5821
MGSEEEKVVVPRNFRLLEELERGEKGIGDGTVSYGMDDADDILMQSWTGTILGPHNTAYEGKIFQLKLFCGKDYPESPPTVRFQSRINMACVNPENGVVDPSHFPMLSNWRREYTMDDLLIQLKKEMMSSQNRKLAQPLEGNEEGRTDPKGLVVKCCVM

SEQ ID NO: 168 - protein, 159 aa, Arabidopsis thaliana
  misc_feature (1)..(159): Public GI no. 28827264
MGSEEEKVVVPRNFRLLEELERGEKGIGDGTVSYGMDDADDILMQSWTGTILGPHNTAYEGKIFQLKLFCGKDYPESPPTVRFQSRINMACVNPENGVVDPSHFPMLSNWRREFTMEDLLIQLKKEMMSSQNRKLAQPLEGNEEGRTDPKGLVVKCCVM

SEQ ID NO: 169 - protein, 158 aa, Arabidopsis thaliana
  misc_feature (1)..(158): Public GI no. 20259984
MSSEEAKVVVPRNFRLLEELERGEKGIGDGTVSYGMDDADDIYMQSWTGTILGPPNTAYEGKIFQLKLFCGKEYPESPPTVRFQTRINMACVNPETGVVEPSLFPMLTNWRREYTMEDILVKLKKEMMTSHNRKLAQPPEGNEEARADPKGPAKCCVM

SEQ ID NO: 170 - protein, 159 aa, Arachis hypogaea
  misc_feature (1)..(159): Public GI no. 71040677
MGSEGSSVVVPRNFRLLEELERGEKGIGDGTVSYGMDDADDIYMQSWTGTIIGPPGTVHEGRIYQLKLFCGKDYPDNPPSVRFQTRINMTCVNQETGVVEPQLFPMLANWQRECTMEDILLQLKKEMSSPQNRKLAQPPEGNEEGRIDQKGLVVKCCIV

SEQ ID NO: 171 - protein, 159 aa, Glycine max
  misc_feature (1)..(159): Ceres CLONE ID no. 540991
MGSEGSTVVVPRNFRLLEELERGEKGIGDGTVSYGMDDADDIYMQSWTGTIIGPPGTVHEGRIYQLKLFCGKDYPDNPPTVRFQTRINMTCVNQETGVVEPHLFPMLANWQRECTMEDILMQLKKEMISPQNRKLAQPPEGNEDGRIDQKGLALRCCVM

SEQ ID NO: 172 - protein, 161 aa, Oryza sativa subsp. japonica
  misc_feature (1)..(161): Public GI no. 50918253
MASRAELPLKQVPRNFRLLEELERGEKGIGDGTVSYGMDDADDIYMRSWTGTIIGPPNTVHEGRIYQLKLFCDTDYPDRPPTVRFQTRINMSCVNQETGMVEPSLFPMLGNWQREHTMQDILIGLKKEMSAPQNRRLHQPHDGNEDQRVEQKGLSLRCVIM

SEQ ID NO: 173 - protein, 160 aa, Triticum aestivum
  misc_feature (1)..(160): Ceres CLONE ID no. 616699
MGSEGSAPVVVPRNFRLLEELERGEKGIGDGTVSYGMDDADDIYMRSWTGTIIGPPNTVHEGRIYQLKLFCDTDYPDKPPTVRFQARVNMTCVNQETGMVDPRRFPMLGNWKREHTMEDILISLKKEMSTPQNRRLHQPHEGNDDQRVEQKGLAARCVVM

SEQ ID NO: 174 - protein, 160 aa, Triticum aestivum; identical to SEQ ID NO: 173
  misc_feature (1)..(160): Ceres CLONE ID no. 677401

SEQ ID NO: 175 - protein, 161 aa, Zea mays
  misc_feature (1)..(161): Ceres CLONE ID no. 220463
MGSEGSSGPVVVPRNFRLLEELERGEKGIGDGTVSYGMDDADDIYMRSWTGTIIGPPNTVHEGRIYQLKLFCDTDYPDKPPTVRFQTRINMTCVNQETGLVEPSLFPMLGKWEREHTMEDILASLKREMSTPQNRRLYQPHEGNEDQRVEQKGLSLRCVVM

SEQ ID NO: 176 - DNA, 633 bp, Brassica napus
  misc_feature (1)..(633): Ceres CLONE ID no. 975428
gtcaaagtagatccggatcatctctatcctcccgacgacggaatcatccaactccggaatggcgtcgaggagaaacgtacggtacgctcagcttccaggggaggaagacgatgaggattacgcaaacggtggtggaaggagagatttcgatcctcgatttgagtatacgccgaaagcatttgatagagtaccgtggaagtctatagcgttagctttgtttcttctgtttcttggttgcttgcttctccttttgacggttttcatattcactggtcacatggaaggagatagctctcagggttacgcgcttcttgttcttggcatccttactttcctccctgggttctatgagactcggattgcttactattcgtggagaggagctgaagggtaccgtttcgcagccattccctcctactgacttcccaagtagctggagagacgttacacgactctgtgcataaactttttttttctttcaaaggaagaatcttagtggagatacgtaatagtatacaaataatagacttgatgaggtgtttcatgaatcactccaaaaggcttggattattatagatagggatactacatagactgttatgtttccattcatctaacttgtgaatcttattg

SEQ ID NO: 177 - DNA, 633 bp, Brassica napus
  misc_feature (1)..(633): Ceres CLONE ID no. 965227
acatatgcttcgtcaaaagtagatccggatccttcttccatcatcggaattgttaagctccgggatctgtaagggttatatactgattaggaatggcatcaaggaggagtgtacggtatgctcagcttcctggagatgatgaagactatgcagatggtggtggtggtgggaggagagatttcgatcctcgttttgattacacaccgaaggcgtttgatagagtgccatggaaatcgataggactagctgtgtttcttctcgtccttggttgtttgcttctccttttggcggttttcatcttcaccggtcacatggaaggagatagctctcaaggatatgctcttcttgtccttggcttccttactttcctccctgggttctacgagacacggatagcttactattcatggagaggagctgaagggtaccgtttcgctgccattccctcttactgacttcccaactagcttatcatctgtgcatagaaaagttgtagttttttttctttatgagcaagtcatattatagaaaatggaatccaatctagactttgagctgtttcatgaatatcagagagatactacatagattgttgatccagtttgtgaatcttattgaatctcgtttgttatc

SEQ ID NO: 178 - DNA, 669 bp, Glycine max
  misc_feature (1)..(669): Ceres CLONE ID no. 635196
ccattactacattgaagcccaaaatgaactgagccaaacacgaacacccattctaccaaaacagccaccatcggacatcgaggccagcaaaaacagttttaggagcaaagaactcaggaacagatatgactactagacgtgttcgttattcccctcttgccacagatgaggatgattatatcggtgataggaatagaccttttgacccaagatttgattatacaccaaaagccttggataaagttccctggaaatccattgcccttgcactgttcctgttattccttggtacaggacttctgtttctttcatactttatcttcactggtcatatggggggagaacgatctcaagcatatggccttttagccctagggttccttagctttctcccaggcttttacgagactcgaattgcatattatgcatggaggggtgctaaaggataccgcttttctgccatccctgattattagagtgttataataaatgaacaatagagctataccttgaaaacttaaattatcaaatttggttgcaacttgtatgcttcaatggcacagatcgaagtaaatggtagaaaagtgtaatcgtactactactcgatacttaagtaattaattcggtgacaaaatttataaaaattatgcattgaagtttgagtttagg

SEQ ID NO: 179 - DNA, 745 bp, Triticum aestivum
  misc_feature (1)..(745): Ceres CLONE ID no. 891349
gtctgcttctcatctctgatcgttcccctcacctctctggctctctcggacaagacttcctccagtacgcaccagccggcaaaggcgcgccctccggcggccggcgaccagcgaccggcgacttacttcaaaggagcacggacagcagtatggcgaccagacggaacattccttattctgttcttcctacggaggacagggatgaagacaatgttgaccgccggttcacatacactccaaaatccttgaggaggatcccgtggaagtcgattgccttagcattgttcctcctcttcctgggaagctctcttctcttcctttcatattttatatccacgggtcacatggagggtgatagctctcaggtgtatggtctattgttcctgggtatccttgcctttcttcctggattttatgagacccgagttgcatactattcatggagaggagcaccagggttcacctttgcggccattccagattattaggctttgattctaactcatctactgggcagtaaggatattgaacagcaagagccaaaatacgaaactttatcgagaatgagatgagcacttattgttatgtttgtactacaaatagctaacatgtaggtgaagtcgccttgtgttgattctatgtcacatgactgcgttgtttcatcaggcaagctacgcatgtgctgcagagctaaagctgctcttttgctttgcttgattgctatgtagattgaacttctggagtt

SEQ ID NO: 180 - DNA, 782 bp, Triticum aestivum
  misc_feature (1)..(782): Ceres CLONE ID no. 1054465
tccgatcgttcccctcacctctctggctggctctcacgtaggagacctcctccagtacgcaccagccggcaagggcgtgccctccggtggccggcgacctacttcaaaggagcacgaacagcagtatggcggccagacggaacattccttattctgttcttcctacggaggacagggatgaagacaatgttgaccgccggttcacatatactccaaaatccttgaggaggatcccgtggaagtcgattgccctagcattgttcctcctcttcctgggaagctctcttctcttcctttcatattttataaacacaggtcacatggagggtgatagctctcaggtgtatggtctattgttcctgggtatccttgcctttcttcctggattttatgagacccgagttgcatactattcatggagaggagcaccagggttcacctttgcagccattccagactattaggcttcgattctaactcatctactgggcagtaaagatattgaacagcaagagccagaatacgaaactttatcgagaatgaaatgaacacttattgttatgtttgtactacaaatagctaaaatgtaggtgaagtcgccttgtgctgattcttgtcacatgacggtgttgtttcatcaggcaagctacgcatgtgctgcagagctaaagctgctcttgtgcttgctatgtagattgaacttctggagttacctaagatagtagttttatggcttaagtacgttaatctacatatatatttttataaaaaaaaaaaaaaaa

SEQ ID NO: 181 - DNA, 837 bp, Zea mays
  misc_feature (1)..(837): Ceres CLONE ID no. 1602143
actcagcctccgccatttcttcatctccaatctccatccttccatcttccgctccctcccctgcttgcgctcgtcgcgcaggcgacctcggcggcgctcgggctcggcctctcagcgacggcgtcagcggcttcccctcctgcctctcgccagtcccctcggctctgttcctctgcgcaccggccggtgaccctgccacaggtacccaagtatggcggcaagacgcaatgttcggtactcgcctcttccttcagaggacggggatgacagcaattttattaaagaagatgttgacctccgatacacttacactccgaaatcgtacagaaggatcccatggaagtcaattgccctggcattgttcctcctcctcctaggaacttcacttctctttctttcttacttcatattcacaggtcacatggagggtgatagttctcaggcatatggtctcttgttcctgggtttcctttcctttcttcctggtttctacgagactcgagttgcttactattcatggcggggagcgccagggtacacttttgcatccataccagattattagcctttcagtttgcgctgctgatgcagtaaggataatgaacaagcaaacaacttcaaactttccttgggaaccaattttagtagtaaaagagttaagcatgcatatgtagtggctcaaatgattgttgtgttaataggatgttattgctttgtcggtcagtaaaagaataattccttgtgatattgtgtaagtgcaaaggttgattttgcaatttcttggtatttccttgtcaaatataacagaaattagtgcaaaaaaaaaaaaaaaaaaaa

SEQ ID NO: 182 - DNA, 659 bp, Zea mays
  misc_feature (1)..(659): Ceres CLONE ID no. 1064651
aaaaccataaataaaagatgtcaaaagattgcggcaaccacggtggtggcaaagaagcagccgtccggcgaatctgcgccgccgttatagccttcatcataatagttctaatcaccatctttctagtatgggtcatactaaggccaactaagccaagattcatcctgcaagaagccaccgtcttcgccttcaacctctcgcaaccaaacctcctcaccacaaactttcagatcacattcgcatctcgtaaccctaactccaagatcggtatctactacgaccgtctccacgtttacgctacgtaccgtaaccagcagataactctcaggacagcaatccctccgacgtatcaaggccacaaagaagacaacgtctggtctccgtttgtttacgggaccgctgttccgatcgcaccgtacaactccgtcgctttgggagacgagcaaggtcatggactcgttcagttgatgatacgcgccgatgggcgcgtgaggtggaaagtagggacgttgatcaccggaaaatatcatatccatgttcggtgtccggctctcatcaatcttgggaacaaagctgctggtgttattgtcggtgacaacgccgttaagtatacgttggttactaaatgcagtgtgaacgtttagaactttgacgccgtt

SEQ ID NO: 183 - DNA, 899 bp, Brassica napus
  misc_feature (1)..(899): Ceres CLONE ID no. 970655
attacataggatgatcggagggcatttacacacaattataatataaatatacacaaccatctttcccacacatatttttttcctcccatcatcactagtcacgaccaaacacaaaaaggaagaaatatatcatatatagagagagaaagaagcacaacagtggcagaaccgtaaataaaagatgtccaaagattgcggcaaccacggtggtggcaaagaagcagccgtccggcgaatctgcgccgccgttatagccttcatcataatagttctaatcaccatctttctagtatgggtcatactcaggccaactaagccaagattcatcctccaagacgccaccgtcttcgccttcaacctctcgcaaccaaacctcctcaccacaaactttcagatcacattcgcatctcgtaaccctaactccaagatcggtatctactacgaccgtctccacgtctacgctacgtaccgtaaccagcagataactctcaggacagcgatccctccgacgtaccaaggccacaaagaagacaacgtctggtctccgtttgtttacggaaccgctgttccgatcgcmccgtacaactccgtcgctttgggagacgagcaaggtcatggactcgttcagttaatgatacgtgccgatgggcgcgtgaggtggaaagttgggacgttgatcmccggaaaatwtcatatccatgttcggtgtccggctctcatcaatcttgggaacaaagctgctggtgttattgtcggtgataacgccgttaagtatacgttggttactaaatgtagtgtgaacgtttaraactttgacgccgttaagttgcttctatcttcttttttattattattatatataatcaaatcattttctatttacaaaaacaaattttacc

SEQ ID NO: 184 - DNA, 1355 bp, Glycine max
  misc_feature (1)..(1355): Ceres CLONE ID no. 465057
acacagagacatttttctttcttagaagcgtccaccacacggaacctcgcaattccttcacacccaccataataataatatcttcattgttcttttcttctttgttaccattaaaaacacacacacacacacacaacaacactctgaaactaacaacaacaataaaagaccatgtcggtgaaggagtgcgagcaccacaaggggaagaagaggaagatcttccggcaagtgttctggtgcttagtggtgttccttttcatcgtgctggtcacaattcttttgatatgggcaatccttagacccaccaaacccaccttcacgctccaagacgtcaccgtctacgccttcaacgccaccgtggcgaacttcctcacttcgaattttcaggtcacgctcatctcgcgtaacccgaacgaccgcatcggtgtttactatgatcgcctcgaaaccttcgtgacctaccggagccagcaggtcacgtaccgaaccgccatccctccgacatatcaaggccataaagagatcaatgtttggtctccgtttgtttatggcaccaacatccccgtcgcgccgttcaatttcctcggcctcagccaggaccagagtaacggtaacgtcctcgtcaccatccgagccgagggtagggttcgttggaaggtcggcaccttcatctccggccgctaccacctctacgtccgctgcccggcgttcatcagctttggaccccgcagcaatggaatcgtcgtcggagagaacgccatcaagttccagattatccaacggtgctccgttagtgtctaaacttaatctcagacaagaaraaatgttaataacatattttctaatacactcttttaaatatgtttattagtgacggaatttagtgatgtattgccttttcgtcactaattctaaagttacaaaaactttagtgatgaatttttcaaaagttacatatgaaatatttcgtcactaaaatttattgaaaattaaatctggaaaattcatggttattttttttatgtgattttatagtttttaataaatttaaactaatcataaaaaatgtgttaaaatgagtgtaataaagaatatgttattgacacttgtggtatgtatactctactcgatccgatccagtgtatttttataggtaccaatttcttcttccactgttgttattgtacgtgtaccttgtgtacgaattggtcgttagatgtttctattttacgttttgctatagaaatgtgaagcagacaaaagagtctcagatatagcaaaattacaagatgcaagatggagactaaatgtaattgagctacgatggcaataaaatttcttgtttcaatttggat

SEQ ID NO: 185 - DNA, 1087 bp, Glycine max
  misc_feature (1)..(1087): Ceres CLONE ID no. 650444
atatatcatttcaagaccttaaagtaccaaacttccaactcttctctcttctctcttcactcttcactcttgtcgtcatttcctcacaaaaccagtgtccgtaacatctccaccttaacgatgtcggtaaaggagtgtcaccaccacaagggaaagaagcacaaactctggagacgcatcttctggggcatagtgatcttcgccttcatcgtgttgctaacagttcttataatctgggcaatcctaaagccttcaaagccaaccttcatcctccaagacgtgaccgtctacggcttcaacgccaccatccccaacttcctcacctcaagctttcaggtcacgctttcctcgcgcaaccccaacgacaaaatcggagtctactacgaccgtctcgacacctacgtcacttaccggaaccaggtcacgtaccgaacctcaatcccaccctcataccaaggtcacaaggaagaggatgtgtggtccccattcgtcttcggcactaacgtccccgtcgcacccttcaacttcgttggtctcagtcaagaccagaccaacggcaacgtcctcgtcctcgttaagattgacggtaaggtacgatggaaagttggtaccttcgtctctggtcactacaacctctatgtaaggtgccctgctttcataacttttggcccccaaagcaccgggattgcccttggcaagaacgccgttaagtatcaattggttcaacgctgcaccgtcggggtttgatgtaaccgccgtccaacatatattcattctgtctttgctctgctccttgctatatttagtgtctatctatatacggtcttttctcatttctttttttttatatacgtattcttgttatcttccactacgtatttcttctgtattgacggatcactttgttgttaggcatacgtgcgtgtctttgttacagcatttggaattgtacattttgctacagaataaactgcacttggaaaaaaaagtatgtaatagagatgaaagcgaaattacaatttgtaacaaagagccaattgaaataaattaaaacgttgtttcaatttggacaaaaaaaaaaaaaaaa

SEQ ID NO: 186 - DNA, 1066 bp, Glycine max
  misc_feature (1)..(1066): Ceres CLONE ID no. 662698
aaagaaaggcaacaagtatttattgatataaaaaatgaagacctcaaagtaacaaacttggaaccccggtcccttgtgatttgctcacaaaaccagtgtcggtcacaccatctctcccttttctccacctgcaacgatgtcggttaaggagtgccaccaccaccacaagggaaagaagcacaagctctggagacgcatcttctggggaatagtgatcttcgccttcatcgtgttactgacagttcttataatctgggcaatcctaaagccttcaaaaccaaccttcatcctccaagacgtcaccgtttacggcttcaatgccaccatccccaacttcctcacctcaagttttcaggtcacgctttcctcgcgcaaccccaacgacaaaatcggaatctactacgaccgtctcaacacctacgtaacttaccggaaccagcaggtcacgtaccgaacctcaatcccaccctcgtaccaaggtcacaaggaagaggatgtgtggtccccgtttgtctacggcactaatgtccccgtcgcaccctacaacttcgttggcctcagtcaagaccagaccaacggcaacgtcctcgtcctcgttaagattgacggtaaggtacgatggaaagttggttccttcgtctctgctcactacaacctcaatgtaaggtgccctgctttcataacctttggcccccaaagcaacgggattgcccttggcaataacgccgttaagtatcaattggttcaacgctgcaccgtcggggtttgatgtcaacgccttccaagatatattgctctgcctcttggtatatttagtgtctatttatatacggtctttctcatttctttttttcatatacgtattcttcctatcttcctctacgtatttcttctacggatcactttgttgttagacatacatatgtctgtcttcgttactgtattttgaattttacattttgctatagaataaatttgtatttggagaaaattctcatgagatgaagcaaaattacaatttatagcaaagtcattttagataaattaaaacgttgtttcaatttgg

SEQ ID NO: 187 - DNA, 1031 bp, Triticum aestivum
  misc_feature (1)..(1031): Ceres CLONE ID no. 632710
ttttttcaaccagacaccgcccctcttcttgctctctgctctcctctcctctcctcttctctcctctcccacacgcgtcctaaagcaagcaagctcggacgccatgagcaaggactgcggcaaccacggcgaggacgacatccggcgcacgtgccggcgcttcctggccttcctcttcttcctggccctggtggtggccgtcatcgccctcatcgtctacctcgtcctccgccccacgcacccgcgcttctatctccaggacgcctcgctccggcagctggacgtgctcaccgccaacgcctccgccgcggcgggcgtgctctccaccgtgctccaggtcaccgtcgcctcccgcaaccccaacgaccgcgtcggcgtctactacgaccgcctcgacgtctacgcctcctacaagtaccagcagatcacgctggcctccgcgctcccgccggtgtaccagggccacggcgacgtcgaggtgtggtccccggtgctctccggcccgaacgtccccttcgcgccctacctcgccgacgcgctcgccaaggacgtccaggccgggtacctcatcctccaggtgaagatcgacggccgcgtccggtggaaggtgggcagctggatctccggccactaccacatcttcgtgacctgccccgccttcctcatcggcaccggcggcaacggcgcgcccggggccaacgggctcaggttccagaccgccacctactgccacgtcgaggtctagccgtccgtccgtacgtgctctgcttcctcttcttcttcgtcatcttcctctgctccgtcggcggcggcgaggtaggtttcttgcgggtggcgcgcgcgcgtgcgcgtagccaagaagatcatcaaggctgaaggccaagggggtggtacatggtggtaataagtgttagcgtcgaggggtataggtgtaaaaaggaaaagagaatttagtgttggttactcctactaaattgtgttttgctcgtttttaattgttggtaaggtatttattccacgtttaattgtcg

SEQ ID NO: 188 - DNA, 1311 bp, Arabidopsis thaliana
  misc_feature (1)..(1311): Ceres CLONE ID no. 19561
gtaacaacgtcacctaaatctctctcacccaatttgtgtttcttctttctgctaaaaggttataatttttgtttcttggtttggtgagaatcttcaagaaactgaaacaaagaaaatggattctagttgcatagacgagataagttcctccacttcagaatctttctccgccaccaccgccaagaagctctctcctcctcccgcggcggcgttacgcctctaccggatgggaagcggcgggagcagcgtcgtgttggatcccgagaacggcctagagacggagtcacgaaagctaccatcttcaaaatacaaaggtgttgttcctcagcctaacggaagatggggagctcagatctacgagaagcaccaacgagtatggctcgggactttcaacgagcaagaagaagctgctcgttcctacgacatcgcagcttgtagattccgtggccgcgacgccgtcgtcaacttcaagaacgttctggaagacggcgatttagcttttcttgaagctcactcaaaggccgagatcgtcgacatgttgagaaaacacacttacgccgacgagcttgaacagaacaataaacggcagttgtttctctccgtcgacgctaacggaaaacgtaacggatcgagtactactcaaaacgacaaagttttaaagacgcgtgaagttcttttcgagaaggctgttacacctagcgacgttgggaagctaaaccgtctcgtgatacctaaacaacacgccgagaaacactttccgttaccgtcaccgtcaccggcagtgactaaaggagttttgatcaacttcgaagacgttaacggtaaagtgtggaggttccgttactcatactggaacagtagtcaaagttacgtgttgaccaagggatggagtcgattcgtcaaggagaagaatcttcgagccggtgatgttgttactttcgagagatcgaccggactagagcggcagttatatattgattggaaagttcggtctggtccgagagaaaacccggttcaggtggtggttcggcttttcggagttgatatctttaatgtgaccaccgtgaagccaaacgacgtcgtggccgtttgcggtggaaagagatctcgagatgttgatgatatgtttgcgttacggtgttccaataagcaggcgataatcaatgctttgtgacatttttccttttccgattttttgctttcgttttttaatttttttttttgtcaagttgtgtaggttgtgattcatgctaggttgtatttaggaaaagagataagaccaaaaaataaattggaacaatggttttgtgt

SEQ ID NO: 189 - DNA, 1603 bp, Glycine max
  misc_feature (1)..(1603): Ceres CLONE ID no. 597624
ccacccaaactcatctatctttgcttccccttagtcaaacaaagtaacacaccatccatcctctctctcttctcttcttctgttctctagtttcctgctcttgtttcttagaatccgtacggtctaatcaacacaacaaaatggatgcaattagttgcctggatgagagcaccaccaccgagtcactctccataagtcaggcgaagccttcttcgacgattatgtcgtccgagaaggcttctccttccccgccgccgccgaacaggctgtgccgcgtcggtagcggtgctagcgcagtcgtggattccgacggcggcggcgggggtggcagcaccgaggtggagtcgcggaagctcccctcgtccaagtataagggcgtcgtgccccagcccaacggccgctggggctcgcagatttacgagaagcaccagcgcgtgtggctgggaacgttcaacgaggaagacgaggcggcgcgtgcgtacgacgtcgccgtgcagcgattccgcggcaaggacgccgtcacaaacttcaagccgctctccggcaccgacgacgacgacggggaatcggagtttctcaactcgcattcgaaatccgagatcgtcgacatgctgcgtaagcatacgtacaatgacgagctggaacaaagcaagcgcagccgcggcttcgtacgtcggcgcggctccgccgccggcgccggaaacggaaactcaatctccggcgcgtgtgttatgaaggcgcgtgagcagctattccagaaggccgttacgccgagcgacgttgggaaactgaaccgtttggtgataccgaagcagcacgcggagaagcactttcctttacagagcgctgctaacggcgttagcgcgacggcggcggcggcgaagggcgttttgttgaacttcgaagacgttggagggaaagtgtggcggtttcgttactcgtattggaacagtagccagagttacgtcttgaccaaaggttggagccggttcgttaaggagaagaatctgaaagccggtgacacggtttgttttcaacggtccactggaccggacaggcagctttacatcgattggaagacgaggaatgttgttaacgaggtcgcgttgttcggaccggttgtcgaaccgatccagatggttcggctctttggtgttaacattttgaaactacccggttcagattctatcgccaataacaataatgcaagtgggtgctgcaatggcaagagaagagaaatggaactcttttcattagagtgtagcaagaaacctaagattattggtgctttgtagcgttacgttactttttttgagttttttttttttttgagttttgtgactgatgaaagaaagaaggtacaagaagaacggcggtgtagtggcatagtggcatcgcaagttgctgcaaaaggtgaattgtatattacttaatattagatgctgaaatattaggtgtaatgtaacaaaaaactgtacaaggagaagaaaaaaggttctaagaaggggagaagctagaagaaaaaaaatgatgtcatcatgggataactgtttaattgtatatgttgataatattgttgaatgttgattattatgttcacggc

SEQ ID NO: 190 - DNA, 1607 bp, Glycine max
  misc_feature (1)..(1607): Ceres CLONE ID no. 566154
attcatctctgatccaaacgaagtaacgaaccaaccaatcaccctgtctttctgacccaaaaacatagtcccaaatcgaacaaaatcaatcaactaactcctccaacgttgttgttattatgcagacaacaagttccaacacctccggcgccacctcgatccgttccggcatggagctcttccaccgcgcgaaggcggtgcgcctccgcagccaccacgacaagtacctcctggccgacgaggacgaggagtccgtcacgcaagaccgcaacggatcctcccgcaacgccaagtggacggtggagctcatcccggagttcgacaacatcatcagactcaagagctgttacggaaaatacttgacggcgtccaaccagccgttactgctcggcgtgacggggcgcaaggtggtgcagagcttgccgcggcggctcgactcctccgtggagtgggagcccgtgcgcgacggcgcgcaggtcaagctcaagacccgttacgggaactttctgagagcgaacggtggggtcccaccttggaggaactccgtgacgcacgatatcccgcataggaccgccacacaggattggatcctgtgggatgtggatgttctcgagattcatgtggtgtcccctgctccgcctccgattccgcactccgattcgcttgatttcgaatcgaatactccttctgctgttaacatcaaatctactaccttctccagacaagagtcgacggattcgaatgtgggttcgccgccaaagaagatggaggggaggactatatactaccacgttgcagaagataatggggatgtggatgatgagaatgtgcagggttattctttgaatttcaaagggaatggggttgagcagttagctcgaaagtttgaggaagagactgggcttgagggagttattgtgtgcactcgaagtcctttgaatggaaaactctaccctcttcgcttgcagcttcctccaaacaatgtcaccatgcaggttgttttggttcttccctcatctaaagtggcaagagaatttgaggagcaaggtatactatgacaagctacattattgttggagtcttgaatctactgaaatgcaaggcctatgtacgttgtgatccaagcgtgtttcaaattttatgtgatagcatttaacaatatgttgcaaaactccacctatttcaagtgagtccattgcatttataaggcagagttcaccaaggtccaatggaggtaacttaaagtttccatggcaaggttcatatcagcaatatcctctattaagtggtgtggaagggttgtagatgattctgatggagatgaaagtctttgttaggcttcctatttattagtgctatagctttaatctttagcctattcttgtggattcagatatttgttgtttctttctctgttggtaattttctgtttttcatgtttttttaaggtgtatcatttgtatagacaacttttatcagaaaactagaggtatattttttattcatatataaatttaaggtattagaagcattttttaatcagataattttttgcctcatttgtttgtatctaatcaagtgatcacatcgaatgattttttttttgt

SEQ ID NO: 191 - DNA, 1422 bp, Glycine max
  misc_feature (1)..(1422): Ceres CLONE ID no. 541790
gaaaaaaaccaaataccacaatcaaacctcttctttcaggtccaattcactcgcttattcttttctttccatacacatgcagacaataagttcgaacaagttcaggactggcatggacttctttcaccgcgcgaaggcggtgcgcctccgcagccaccacgacaagtacctcctcgcggaggacgacgaggattcggtgacgcaggaccgcaaaggctcctcgaaaagcgcgcggtggacggtagaatacgtggctgaatacgataacataattcgcctgaagagctgttacggcaagtacctcaccgcctccaaccaccgcttcctcttgggcatgaccggccaccaggtcctccagacgcttccttcgaggctagactcttctgttgagtgggagcccgtcaaggaaggagggcgcgtgaagctcaagactcgttacgggaattttctcagagctaacggcgggttgccgccgtggagaaactccgttacgcatgatattccgcacaggaccgccacgcaggattggatcctatgggacgtggatgttgttgagatttatgtgaactctcccgcgagtcatgatagaccctccgctccgccgttgccggttgaggactctgttcccatactaacgagcgcacaggtgccgccgccgccaactgtttctgctagcttttcaaggcaacagtccaatgatttgtcacctaaggtggaagggagaactatatactaccatattgctgaagacaatggagaggtgttggatgaaggcgtgcaggggtattctttgattttcaaagggaatggagttgaagaattgactcgaaagtttgaggaagaaacagggctagagggaatcattgtgtgtaatcggagtccattgaatggaaagctttatcctcttcgcttgcagcttcctccaaacactgtcaccatgcgggttgttttggttcttcccttgtcaaatttggcaagagactttgaggaacaaggcttactgtgagaagccttagagttttccaacgaagacctatgtatatgtactgatcgaagccagttcagaatttaggaattaatgtgttctagtccattgcatttattaagcagagttcatcgtggtcctgtgtgggaagcttagcgttgtcatgggatgggtcataaatgaaatatcctttcatcaatgacatgtgctataggcgattctgatggtggattttgttggggcttctttttcattaactccagctttaatataagcacatgttgcatgtggattccgggaattttgttgtttgatggtttatgtaattttatgtgcttcatgattttttctctaggtgatgtacggcatgtaaatggtataagttttgtcagcaatataaattttcttttatcagatacaaaaaaaaaaaaaaaaaa

SEQ ID NO: 192 - DNA, 1312 bp, Zea mays
  misc_feature (1)..(1312): Ceres CLONE ID no. 218121
atgcgagtccagattccatcgcctaccttgaaataacccgcctcgcctcggcttgcaaccgtcccattgcattgcaaggaaggaacacctgctctgctccgctctcgcccctcgccctccaagcaacaacccgccggctgtctgccgtcgccgccgcgccaccacaacaccagtccaccaccacgcggcggacgccacgacgatggagctctttcctcacgggggcttcgtgcggctgcggagccgagcgcgggacaagtacgtccacgccgacgtggacgcgagcggggtctccctgcgcccgctcggcgccgcaccctctgtgaacgcggtgtggaaaccggagcgttggcccagcgaaggggacttcttaatcctccagatcgtcgcctacggccggtacctcgctgtctcggacaaggacgcgccgcccggccaccgcggcgtccgcgccgtccagcgcgactacaactcgccgcgcgcggaggctcccttcttgtggacggcctttagggtggatgcccaccagaactacgtccgcttgcacaaccaccagcgctggctccgcgccaacggcaggcatcgctactggaacaacgtcgtcaccatcgataccaggaacgccagcctgaccacgatgatgcagtggagggtcgaggaaatcccagtgagcccggagccgctaccccttccgcctccacctcaacaacctacgattcatgtaagccgtggcctattcaagcggcgggccaaggtgcatccatcgggccggatgatacagcacgtgcggatggacgatgatgggtttattcttgacaattggcctgatttcaacttcgacgactactccgtgtccaacctgagggccgaggtggcgctccgtcaggatgacgagaacatcacgttgtgcytgcgggccggcaagcatgcgctcttaatcccgcttatcacagacctgcctcataacacagatcccctggacatcatcgtgatgcccgccggatcaccaggtttagatgcgttagagtacccacagtttgatgcgccagaaccgtagaggagtaccggagtagagctgttaaactttggcactttgcaattcatctttgggaagcagtgagcctttaagcatcgacgcccctgatatatacagtgacatgagaattgattgttatcttttgttaagatagtttcagcaattttttttctgtaatggtggttagatttgatgcgatccactgaccggatgcggtttgagatgacatttcagtttcagaaacttaaaaaaaaaattctgaaaccattatgtagg

SEQ ID NO: 193 - DNA, 1336 bp, Glycine max
  misc_feature (1)..(1336): Ceres CLONE ID no. 1043576
gcgcgttgaagagcgaagaaggaagaagatgtcgggtatgggagatgggtacgtgggcaccgcccaagacgcggtgaggattcgacggttggagaagcagagagaagcggagcgtcgcaaaatccaagagctgaaaaccaagtctgcctccgccaaggaccaacccggtcttctccaattcggttccagcacttccgagattctagagactgcctttaagaaggaaactgtgggtttggtcactagagagcagtatgtagaaaagagggttaatattcagagcaaaattgaggaagaagagaaggagaaacttcagaagcaacaacaagaagaggaggagcttcaattacaaaagcggaaaaagaggaagatcaggggcaattctcgattgtcctttgctgaggatattgacaatgaaccccaagaggacgaaccacatcacagtaaggataatctggaggcaaatagattacggtgtggtaagcttggcaaagaccctactgttgaaactagctttctacctgacagtgagcgagaggctgaggagcaagctgagcgtgaaaggctgcgcaaacagtggcttcgtgagcaggagcaaattcgaaatgagcctcttgaaatcacatacagctattgggatggaactggccacaggcgtgtgatccaggtacgcaagggtgacagcataggagagtttcttcgagcagttcaacagcaacttgctcctgaattccgagagattcgaacaacatcagtagaaaatttgttatacgtgaaagaagaccttatcattcctcatcaacatagcttctatgagctaattgtgaacaaagctagaggcaaaagtggaccgctttttcattttgatgtgcatgaggatgtacgaacaattgctgatgccactatagagaaggatgagtctcatgctgggaaggttgtagaaaggcattggtatgaaaagaacaagcatatatttcctgcttcaagatgggagatatatgatccaacgaagaaatgggaacgttatactatccacggggattgatttgaggctgccaaataatttgcattagcatttcctgtattgacatttaatttggtcatgcgtttgtacgccttggtgataattatgctattgttagtcatttcagtgtttagaatgactaggttttgtgtttctgttattcattacagacaacatggacaattagtcagatctatattacagtggatttcgtgtcaaataagctaactttaatctatttactagctaacgtacttgtttaacgaaattaagcatttggtaattaagctttcaacaaaaaaaaaaaaaaaa

SEQ ID NO: 194 - DNA, 450 bp, Brassica napus
  misc_feature (1)..(450): Ceres CLONE ID no. 1079973
aaactcaantcatacaaaacaaaacaaacatcgaccaataaaggctaaaagcttaattcacaaaacaaacatcgatattayggacaaggtgatgagaatgtcgtcagagaaaggagtggtgatcttcacgaagagctcatgttgtctctgctacaccgttcagatcctgttccgtgaccttagggttcaaccaacgatccacgagatcgacaacgatccagactgccgcgaaatcgagaaggctctcctccgtattggctgttccacggcggtgccagctgtcttcgtaggtggcaagctcgttggttccaccaatgaagtcatgtcccttcaccttagtggttctctcgtccccttgatcaaaccctatcagtccctcctttaccaggccaattgaaccaactcgatcgagatccttcatataattaatcttattatctagccagtgac

SEQ ID NO: 195 - DNA, 399 bp, Brassica napus
  misc_feature (1)..(399): Ceres CLONE ID no. 953083
aaacgaaacaaagaaacataaaccagagctaaaagatacataagttctcgatctttggagggagccgtccgatcagaatctgatcatggacaaggkgatgagaatgtcatcagagaaaggagttgtgatcttcacaaaagactcatgctgtctttgctacgccgtgcagatccttttccgtgaccttagggttcaaccaaccatccacgagatcgacaatgatcctgactgccgcgagatagagaaggccttagtccgtcttggctgcaccaacgctgttcctgctgtttttgtcagtggtaagctggtgggttcaaccaacgaagtcatgtcgcttcacctaagtggctcccttgttccattgatcaagccgtatcagtcatttcataactagaaaat

SEQ ID NO: 196 - DNA, 645 bp, Triticum aestivum
  misc_feature (1)..(645): Ceres CLONE ID no. 1030898
aacgaaacaaagaaacataaaccagagctaattaaagatacataagttctcgatctttggagggagccgtcagatcagaatctgatcatggacaaggtgatgagaatgtcatcagagaaaggagttgtgatcttcaccaaagactcatgttgtctttgctacgccgtgcagatccttttccgtgaccttagggttcaaccaaccatccacgagatcgacaatgatcctgactgccgtgagatagagaaggccttagtccgtcttggctgcaccaacgctgttcctgctgtttttgtcagtggcaagctggtgggttcgaccaacgaagtcatgtcgcttcacctaagtggctcccttgttccattgatcaagccgtatcagtcatttcataactagaaaatgaatggatccttaagaaaaggtaattggtatgttgattagatagtaaataatgatggaaatattacacgtaaatgtgtatcatgttcttatatatatagctaatttttatattttgctttgctaatcatgtcaaaaacatttaattagctagctttgtattttccagcttaatcaatcagtggatgtactgatgtgttatctattgtaatggagaaactatatatcatatgttatgttgctttt

SEQ ID NO: 197 - DNA, 463 bp, Brassica napus
  misc_feature (1)..(463): Ceres CLONE ID no. 940212
aaacgaacaaagatacataattcaaatctaaagatacataatatctcgaccgttgggagccgcaagatcagaaactgatcatggacaaggttatgagaatgtcatcagggaaaggagttgtgatcttcaccaaaaactcatgttgtctgtgctacgccgtgcagatactttttcgtgaccttagggttcaaccaacaatccacgagattgacaacgatcctgactgcctcgagatcgagaaggccttagtccgtcttggctgccccaacgcagttcctgctgtttttgtaagtggtaagctggtgggttctaccaatgaagtcatgtcgcttcacctaagtggctctctcgttcccttgatcaagccgtatcagttatttcataactagaaataaatggatccttaagaaaaagaaagataattgttgtatgttgagattggatagtaaataatgatgganag

SEQ ID NO: 198 - DNA, 606 bp, Brassica napus
  misc_feature (1)..(606): Ceres CLONE ID no. 1070065
aacgaacaaagagtcatacatcaaagctaaagataaataatttctcgaccgttcaaagccgtttgatcagaatctgatcatggacaaggtgatgagaatgtcatcagagaaaggagttgtgatcttcaccaaaaactcgtgttgtctttgctacgccgtgcaggtccttttccgtgaccttagggttcagccaaaaatccacgagatcgacaatgatcctgactgccgcgagatcgagaaggccttagtccgtcttggctgcgccaacgcggttcctgctgtttttgttagtggtaagctggtgggttcgaccaacgatgttatgtcgcttcacctaagtggatctctcgttcccttgatcaagccatatcaggcgttccataactagaaaataaatcgatcactacgaaaagataatggtgtgaaagataacacgtaaatgtgtgtcatgttatgatatatatataactaatgtttatgttttgtttagctcatgtctaagaaattaattagctagcatttgtaattccagcttaattaatcagtggatgtactgaactactgatgtgtatctattgttatggatgaaattattttttatatgtt

SEQ ID NO: 199 - DNA, 500 bp, Arabidopsis thaliana
  misc_feature (1)..(500): Ceres CLONE ID no. 125679
atatacttcttcttcttcaccttatgcaagataatggacaaagttatgagaatgtcgtccgaaaaaggggtggttatatttaccaagagctcctgttgtttgtcctatgcggttcaagttctcttccaagatcttggtgttaaccctaagatccacgagattgataaggaccctgaatgccgagagatagagaaggctcttatgaggctagggtgttcaaagccggtcccagccgtcttcattggtggcaagctcgttggttcgaccaacgaagtaatgtccatgcacctaagcagctcgctcgttcccctagtgaagccatatttatgttaaacaacaacgaaggagtatttatgatattaattagctatgtatatgttattcaataaggaacaaaattgagccaaatctttgtaatgtgttttttggtattattattggttgtataacattgggaaagtgtacgtataattataagactgttatattgattcgaaggt

SEQ ID NO: 200 - DNA, 669 bp, Arabidopsis thaliana
  misc_feature (1)..(669): Ceres CLONE ID no. 39560
caacactgagcttaatactgtagtacacacacacacacacacacacacacacacaaaaccctcttttcttcaaacaggaaccccaaaagcgaggtttaatctcaggcgttttcagttctaaatctctttcaatccacaagagaaggaagattttgtttcttcttctccaagaaacaatccctagattgatcgagacctccttcaagaaaccatggacaaagttgtgagaatgtcgtcagagaaaggagtggttattttcagcaagagctcgtgttgcatgtcctatgcggtccaagtacttttccaagaccttggggttcacccaacagtccatgagatcgataaagaccctgaatgtcgtgagatcgagaaagccctaatgaggttagggtgttccacgccggtcccagccatctttgtgggtgggaagctcattggttcgaccaatgaagtcatgtcgcttcacttaagcggctcgctggttccgctagttaagccgtttcaagccaatctatgttaaaaaggtgttctaattttctatactacaaaaatgtatttcaataagaaacacaaatcttatagcctatgcaacctttgtaatgtagctttatatatatattgtttgttctgtaatttcaggtaatatccaataataattgactaatttcc

SEQ ID NO: 201 - DNA, 508 bp, Brassica napus
  misc_feature (1)..(508): Ceres CLONE ID no. 871147
aggcaacactgagcttgatactataggatatacaacaacaaaaactcttttcttcacccaaaagctaggtttatttcatgggcttttggttttaaatatctttcaatctacaagagagaaagaattttgtttcttcttctccaagaaacaagccacagattgactgagacttcctttaagaaaccatggataaagttgtgagaatgtcttcggataaaggagtggttatattcagcaagagctcgtgttgcatgtcctatgcggtccaggtcctcttccaagacctcggggttcacccgactgtccatgagatagacaaggaccctgattgtcgtgagatagagaaagccctaatgaggctcgggtgttccacaccggtcccagccatctttgtggatgggaagctcgttggttcgaccaatgaagtcatgtcgatgcaccttagcggctcgctggttncgctggttaagccatttcaagctaatcctatgttaaaatgtgttctaac

SEQ ID NO: 202 - DNA, 629 bp, Glycine max
  misc_feature (1)..(629): Ceres CLONE ID no. 510704
aacctcaaccctcatcatagttttttctcctcctacttagagccattagatcgaattcgatcatttcggatttgatctgatggttttggacttctaattagtctaagatggacaaggttacgaggttggcaacagaaaagggtgtggtggttttcaccaagagctcttgttgcctctgctatgcagtcaacattctgttccaagagcttggagtgatccctgtggttcatgaaattgacaaagaccctgagggcaaggaaatagagaaagctataacaaggttggggtgtcctacacctgttcctgcagtcttcatcggagggaagctggtggggtccaccaatgaagtcatgtccctccaccttagtggttcactcactcaactgctgaaaccatatcgagctttgtcttaaagaagggtgcacatgcaaattaaattaaaacatgttcctgctaccttaattggaagaataaacatgcagtatagtagtgtaactactaagtctataatattggatagtctatgcagtgatgcatgtgtgatgtagttaattaatttctagcatttgattattagctatatataattaatgcaatgtcataattattcttaatgaagttctatttat

SEQ ID NO: 203 - DNA, 1072 bp, Glycine max
  misc_feature (1)..(1072): Ceres CLONE ID no. 525163
ggagaattcagagtgtagaatccatttacatcagaagaattcgtcagtgaaagaatcgttttcttcagatctcacaattagagagcgatggaatatctagggattgatctaagctgcgccgttggatctctccgccacggccagttcccggagaaggattgcttgctccctctcatttccaaactcctcggctacgccatcgtcgccgcttccaccaccgtcaaacttccccagattatgaaaattctgaagcatcagagtgtgagaggccttagcatgatatccttcgagcttgaagttattggatataccattgccttggcttactgtctccacaaaggccttccattttcggcttacggagagttgttgtttcttttgatccaagctttagtattggttgccataatctactactattctcgacctttgcatgcaataacatggatccgggcattgctatattgtgctgtagcacccactatcttagctggtcaaattgatcctctactctttgaagctctgtatgcatcacagcatgcaatctttctctttgcaaggatcccacaaatatggcaaaacttttctaacaaaagtacaggtgaactcagcttcataacttcttttatgaattttggaggttcaatggttagagtattcaccaccatccaagaaagtgcaccaaaaagtgttctattgggctatgcaattggtgtcgcaacaaattttaccatcttgagtcagattatagcataccaaaagcctcaggttggaaaggagaagaaaacaaagtaatagaaccgtatcaggttgtgtatatctcatcatccgggccaaccctctgcaggtgccacggcatccagtccatacacagaattttgtcagtcttcaatcgaacattaccttatttcagatttcaactgctgctaggtgattattatcatttttattttggactgtaggcagcagtgtaaaagtaacaggtgattattatcatttttattttggtacctgagatacttgagcttcatgattttatcgttatttatcaaagctaaaatttaaaggatgc

SEQ ID NO: 204 - DNA, 1202 bp, Zea mays
  misc_feature (1)..(1202): Ceres CLONE ID no. 242812
gacgtccagcccgtcacaaaaggcccatctcactgaagagatacgctggtcggctggtgggcgcgtggcttgtgggccgtgggcttcttcgctgttcgtcgtctcgtcaccactcaccattgtcgacgcaagctgcgactgaacctccgtggcgacggaggagacggccatggtggcagcgatggagctcgagatcctaggcatgaatttcgggtgcgtcctcgcggctctgtccgacgccaagatccccgacaaggactgcctgctcccgctcatctctaagctgctcggctacgccatcgtcgccgcatccaccaccgtgaagcttccccagatactgaaaattttgaagcatggaagtgttagaggactaagtgtggcatcctttgaacttgaggttgtcggctacacaattgctttggcatattgtattcataaaggacttcccttttcagcttatggggagctagcttttctgttaatccaagcaattattttggttgcaatcatttactattactcaccaccaatgggaaccaaaacatggatgaaagctttattatattgtggaatggctccaacggttttggctggaaaaatagatcctgctctttttgaagtcctttatgcttcacagcatgctatcttcttttttgctagagttccacaaatatggaagaattttacgaataaagggaccggcgagctaagcttccttacctgtttcatgaactttgctggttccatcgtaagagtttttaccagcatccaggagaagactcccttaagtgtgatcatgggctccgcaatcggcatcgtcatgaatggtacgctcttgggtcagatagtactgtaccagaagcccgctccgaagaaacagaagaaagaagattaagcaacatggagaagctgcggaaagttcttcctggttgtttaggattctgttgcaacactcgctccacatctgaacaaatggcaccagtataggaagttgttggcaacttgttacgagaaaagaacatagttggactattgctatataacaacatttctgtgctgttgtttttagcacttagtagtgtaccatgacttgagtagttcatacaactgcctttagatattgactgcaagctgctgctgatgtgttttcctttctgctgtgccgttccatattttagtttgagttaaatgcatgaaagttt

SEQ ID NO: 205 - DNA, 1338 bp, Zea mays
  misc_feature (1)..(1338): Ceres CLONE ID no. 243125
acggtcttgacgcggcgtggtagaagagcgtagaaattgaggaagccgctggaattctttgtttgtttgtgggatgctggtggattcgcgcgatccttgctgctgcttgagtgtagagcacgaactcttgcagttacttcctctctcaagagcttcccctgctaaacaaaactctagcttctcggtcctgacctcaaggctggctggctggacgttgatcagcgaatatgatctcagcactccccgtgctctgctatggggtcacagctagtgcgttgcaatcccaatcgatccaccaaataacatcctctacaagtcctgcaccagctttcttcaaactatcaagggacccctcatccttgacaatttgtagcccacaaatacaggcaataagaatccaatcactgcggggcaaccaaaatttgaaggccatagagatgagtgaagggaatagatggggctcagggatgaagctggatgtcatgggcatgacaattggatgcttcatatcgtgtccactgcatacgaacaacctgctgcctttcatctctaagatcctcgggtattccatcattgctgcttccactgttggcaagctccctcagatactgaaaattttgaagcatggaagtgttagaggactaagtgtggcatcctttgaacttgaggttgtcggctacacaattgctttggcatattgtattcataaaggacttcccttttcagcttatggggagctagcttttctgttaatccaagcaattattttggttgcaatcatttactattactcaccaccaatgggaaccaaaacatggatgaaagctttattatattgtggaatggctccaacggttttggctggaaaaatagatcctgctctttttgaagtcctttatgcttcacagcatgctatcttcttttttgctagagttccacaaatatggaagaattttacgaataaagggaccggcgagctaagcttccttacctgtttcatgaactttgctggttccatcgtaagagtttttaccagcatccaggagaagactcccttaagtgtgatcatgggctccgcaatcggcatcgtcatgaatggtacgctcttgggtcagatagtactgtaccagaagcccgctccgaagaaacagaagaaagaagattaagcaacatggagaagctgcggaaagttcttcctggttgtttaggattctgttgcaacactcgctccacatctgaacaaatggcaccagtataggaagttgttggcaacttgttacgagaaaagaacatagttggactattgctatataacaacatttctgtgctgttgttttt

SEQ ID NO: 206 - DNA, 1273 bp, Triticum aestivum
  misc_feature (1)..(1961): Ceres CLONE ID no. 687022
cacgagaccattacctagaacatcctaatcgaaaagcacacggagcagcaaccgaaggggggaagaagagccgtcgccggagaagaccaaggggaagagcctctgccgccgccagagctagcgtcaccggtgcctcgccgcccctccgtcggcgcgctgccctccccttggtccgctgcagccatcatctcctctctgcaccttcagccgggagcccatccggagcagctgtagattggatgtggagattggcgaatctggatacgatggagctggagatcctgggcatgaacttcggctgcgtcctctcggcgctgtcggacgccaagatcccggagaaagaatgcctgctcccgctcgtctccaagctcctcggctactgcatcgttgccgcctccaccaccgtcaagctcccgcagatactaaagattttgaagcatggaagtgttagaggacttagtgtagcctcctttgagcttgagctcattggctacacaattgctctggcatattgtattcataaaggactgcccttttcagcttatggagagctagcttttctgctgatccaagcaattatcttgattggaatcatttactattactcgccaccaatgggaagcaaaacatggatgaaagctttgctatattgtggactagctccaactgttttggctggaaaaattgatccaggtctttttgaaatcctttatgcttcacagcatgccatcttcttttgtgctagagtaccgcagatatggaaaaatttcacaaataagagcactggcgagctgagcttcctgacttctttcatgaacttcgctggttcccttgtaagagtttttaccagcatccaggagaagactccgttaagtgtacttatgggctctgtaatcggcatcgtgacaaacggtacgattttgggtcagatagcgatgtaccaaaagcctgtgccaaagaaaggaaagaaagaagaatagggcgtgagaactcttgctgctggtttacaattttgctccagggctattcttggagcaacatgcaccacttaccaggacttcatgacaaaaacagtagggagaaaattcatgcagacttgggtgatgatggaaacaataggatatttcttggtttgtaatttttcgaagtatcgtaaaatgactggctgtgtagttttcaattgccaccttggttagttttctctgtgcaactgtttaatgatgctagaaagatgtatatccatttttatgtttgaatattatttgttgttttt

SEQ ID NO: 207 - DNA, 401 bp, Brassica napus
  misc_feature (1)..(401): Ceres CLONE ID no. 1064435
agaattttctccgctgtggttgaaacttttacatctaggaaggaaaaagagaagaaatatgtctcagactgtggttctaaaggtggctatgccatgtgagggatgcgtgggagctgtgaagagagtcttaggcaaaatgcaaggtgtggagtcatttgatgtggatctgaaagaacagaaggtaacggtgaaaggcaacgtggagccagacgctgttctgcagacagtctcaaagacaggaaagaaaacaagtttctggggagctgatgaggctgaaaccgccaaggcctaaatataacaagcaataaaagcatcctacatgtcataataactctgttttagcagagattcccaaataaaatagaaatcatatgtaatacaaagtcaataaatctaatctc

SEQ ID NO: 208 - DNA, 558 bp, Triticum aestivum
  misc_feature (1)..(574): Ceres CLONE ID no. 622673
aaaagcaatcacttgcacgccaccgctctgacctccgtacgcatttattcacccctctcccggcagttccttctcccatcatcctccaacccctccgactcgcggcacgttgcctttcccccccgcgccctaccctcgccgtccgcggatcagccgatctcgcacgcaatgtctcagactgttgaactcagggttggcatgtcatgtgagggatgtgttggagctgttaagcgggttcttggcaaaatggaaggtgttgagtccttcgatgtagacatcaaggagcagaaggtcactgtgaagggtaatgtgacgccggatgctgttctgcagactgtttcgaagacaggcaagaagacggcgttctgggaggctgaaccctcagcggcatctgctgtttcgtcctaaaaaggcacctctataatatgtaaactgtgaggtttgatgtagcaatggcccagttgaaatgctttgcaaggacatgattttctggtgggacggtggtatgtgcaccatgaatttactgcaaataatagcaaactggtaatttgtttgtgc

SEQ ID NO: 209 - DNA, 588 bp, Glycine max
  misc_feature (1)..(588): Ceres CLONE ID no. 625242
acccgtctcatctcacgccacccaaagttgtctttcgacctttctctctcaaaacctttgcttttgcgtggcctatatatatataaaactaacttccccaaaacccaatcatatatcatttcatttctttttttctcgtccatcccgtaaccatgtctcagactgttgtcctcaaagttggtatgtcatgtgaaggatgtgttggagcagtgaagagagttttaggaaaattggatggtgtggaatcatatgacattgatttgaaggaacagaaggtggtagtgaagggaaatgtccagccagacacagttcttgcgaccgtttccaaaactgggaagaagactaccttctgggaaggtgaagcagcagcatccgaaactagcacagcaactgcctagaacatttcgctctattctgttcagaagaagtagctgcgtaaatcaaatactattttatggacttgaaataacgatgggacttgktatctggtgtattttagtgtgtcmcaacaaagattgctgtatatgactgctttcaacatctgtgctactgtattaatgtgcttttattgagagtttaacttccttt

SEQ ID NO: 210 - DNA, 590 bp, Brassica napus
  misc_feature (1)..(590): Ceres CLONE ID no. 944316
atatcctcacccaactccgtccctctctcccggcccagatagatctttcccccgtcctccctcgccttcgccttcgccggcaggagatttccaccaccaccgccgaaccgccccgaccctcctcccctctcgcgctatggcccagactgttgtgctcaaggttggcatgtcctgtgagggctgtgttggagctgtcaagcgggtcctcggcaagatggaaggcgtcgagtcctacgacgtagacatcatggaacagaaggtcaccgtgaagggcaatgtgacacctgacgccgtcctgcaaactgtttccaagactggcaagaagacttcgttytgggaggctgaggccgtgacaagtgaatccgccacacctgccggtgccaccgcctgacaggttcgatgatctggaagtgcccggcaaggcggcaaagaaaaaaaatgtgaatgtatttagcttggatctgttacaaataattgcaaactgctagctcggtctgtacattttacagttgctattcgtgtactgtgatgttctcgtaagtttggggagatgataaaccaatatttgtatgatatatgtgtcgtgttcc

SEQ ID NO: 211 - DNA, 960 bp, Glycine max
  misc_feature (1)..(960): Ceres CLONE ID no. 540991
aacagaccttctacttttgcgaacagctgttagacttgagttgagtcacagtcatcgtctgttcaggatcatattcacgccccctctctctctctctctctctctctctctcttactttgtgtctttctatctctctcttcaattccaaaaccctacccccatccaattccaattcaccctcaatcatgggttctgaaggttccactgtcgtcgttcctaggaacttcagattattggaagagcttgagagaggtgagaagggaattggagatggaactgttagctatggaatggatgatgctgatgatatctacatgcagtcctggactgggactattattggtccccctggtactgttcatgaagggcgtatataccagctgaaattattttgtggcaaggattatccagacaatccaccaactgttagatttcagactaggattaacatgacttgtgtcaaccaagaaactggagtggttgagccacatttgtttcctatgcttgctaattggcaaagagagtgtacaatggaagacattttgatgcaattgaagaaagaaatgatatctccacaaaatcggaagctagctcagccacctgaaggcaatgaagatggcaggattgaccaaaaggggcttgctctgagatgttgtgtcatgtaattcaggtgagaaccaaagaataataatgtaaataattcaatagataatccttttgtgggttaactcatttataaatgtaattatggaccattcgcttatgtaacatggagcaattaaagagcgactacgaatccttgaaaatttatagtcaatcgtgttgaagctgattttactgtggtttctaatttggaagtgtgtatacattcatctgtgtaggtgtgaaccgtatgcacactttgatcctaatattgtgggataacattcaatttatcattgaaaaaaaaaaaaaaaaaa

SEQ ID NO: 212 - DNA, 918 bp, Triticum aestivum
  misc_feature (1)..(918): Ceres CLONE ID no. 616699
ttcgctccggccccagtccagtactgtgcgcgcatctctcctcctttttctccccccctgagccggaaaaaggctttctttgcttcacgcggggctccggatattcgtgtcctcgcggctcagccaccgcggaatccatccagcgcagctcaggggggaagaggaagaggcggccgaagctggaggtggaagaggggctcgccatgggctccgagggatccgcgccggtcgtcgttcccagaaacttcagacttttggaagaacttgagagaggtgagaagggcattggtgatggaactgtgagctatgggatggatgatgccgatgacatatatatgcgctcctggacaggaactattattggtccacccaatactgttcacgagggacgaatctatcaattgaagttgttctgtgatacagattatccagacaaaccaccgactgtccggttccaggctagggtcaatatgacatgtgtgaatcaagaaactggaatggttgatccaagacgatttcctatgcttggaaactggaaaagggaacatacaatggaagacatcctcatcagccttaaaaaggagatgtcaacccctcagaaccgcaggctccaccagcctcatgaaggcaacgatgatcagagagtggagcagaaagggctagcagctagatgtgtcgttatgtaagatacgcgcattgcaatgtacagtgttacactgagtagcatcagaatgcagaagccgaggaaacccaggaggaaatcatgtaatttgacagtcgctagtcgctactttcgatcgaaactgctctattgccagaataaaatgccaatgacttcgttaatcagtctgcacagtggtgtaaccgtttatccctcatttagcaatatcaagagccatgtgcctgctgtttctttcg

SEQ ID NO: 213 - DNA, 842 bp, Triticum aestivum
  misc_feature (1)..(842): Ceres CLONE ID no. 677401
tccgctccggccccagtccagtactgtgcgcgcatctctcctcctttttctcccccctgagaggctttctttgcttcacgcggggctccggatattcgttgtcctcgcggctcagccaccgcggaatccatccagcgcagctcagggggaagaggcggtcgaagctggaggtggaagaggggctcgccatgggctccgagggatccgcgccggtcgtagttcccagaaactttagacttttggaagaacttgagagaggtgagaagggcattggtgatggaactgtgagctatgggatggatgatgctgatgacatatatatgcgctcctggacaggaactattatcggtccacccaatactgttcacgagggacgaatctatcaattgaagttgttctgtgatacagattatccagacaaaccaccgactgtccggttccaggctagggtcaatatgacatgtgtgaatcaagaaactggaatggttgatccaagacgatttcctatgcttggaaactggaaaagggaacatacaatggaggatatcctcatcagccttaaaaaggagatgtcaacccctcagaaccgcaggctccaccagcctcatgaaggcaacgatgatcagagagtggagcagaaagggctagcagctagatgtgtcgttatgtaagatacgcgcattgcaatgtacagtgttacactgagtagcatcagaacgcagaagctgaggaaacccaggaggaaatcatgtaatttgacagtcgctagtcgctactttggatcgaaactgctctattgccgaaataaaatgccaatgacttcgtcaaaaaaaaaaaaaaaa

SEQ ID NO: 214 - DNA, 958 bp, Zea mays
  misc_feature (1)..(958): Ceres CLONE ID no. 220463
atctgcagcagagcagttcctttcttgctctcctccgctagtccggtaccctcgacctcggcgtcccaccgaagacgacctcacgcggagctgggaatattccttcgcctccgtgctccctttagcaccaactgtcacggccgcttccagcacgcatcattctcgtctcccgtctcgagagattgcagggggcggcgctcgaagctggaggaagcggacgggttcgcgatggggtccgagggatcctcgggtccggtcgtcgttcccagaaactttaggctgctagaagaactagaacgtggtgagaagggcattggtgatggaactgtgagctatgggatggatgatgctgatgacatatatatgcggtcctggacaggaactatcattggtcctcccaatactgttcacgagggacgaatctaccaattaaagctgttttgtgatacggattatccagataaaccaccgacagtccgattccagactcggatcaatatgacatgcgtgaaccaagaaactggattggttgaaccaagtctatttcctatgcttggcaagtgggaaagggaacatacaatggaggacatcctggccagcttaaaaagggagatgtcaactcctcagaaccgcaggctgtaccaacctcatgaaggtaacgaagatcaaagagtagagcagaaagggctgtcccttagatgtgtcgtcatgtaataatgtacatttgaattgagcagcgagatgccgagcttgagcgaaccctcttagggcatgtttaggaacaatcccctccaattcccttgctttcaaagaagccctgtaggaaccctcctgtagctcaccttaatcgcaataaagtgtcgatgactgttaaattgcctgcacggctgcacaccatgtaacagcaaccgattcgattttctgtgaaggacaacgtgttcagtgtcggctgttccac

SEQ ID NO: 215 - DNA, 640 bp, Gossypium hirsutum
  misc_feature (1)..(640): Ceres CLONE ID no. 1899078
gaagtttcaataactctgattctttattcgtttattgtgcagatttgcgatcaaaattcttctattctcctctacgaatctggtttatccgcaaaggatcctctaggagacagttgactgtttaactattatgtcgtctaggcgtcatgttcgttacagtcctttagctgttgatgaagatgatgattatcatggtggaaggcgtttcgacccaaggtttgattattcaccaaaagcctttgatagagtcccatggaaatccattgtccttgcagtctttctgctttgtctggggtgcttgcttctttttctttcgtttttcatcttctcggggcatatgggaggagagaagtcgcaggcatatggccttctagtcctcggaattcttactttccttccaggtttttacgagactcggatagcatattattcttggaggggtgctgaaggatacagctttgcttccattccagattattaagacttgaggtttgtcaagggatgcatcccccttccctccgccgcctcctcttgtattgaactaaatcgtaacattatagttaaagagattgtttaaagcaaccttacaatgctgtaaattcatgaattttgagtgcaatctattcgttactttaatcgg

SEQ ID NO: 216 - protein, 116 aa, Gossypium hirsutum
  misc_feature (1)..(116): Ceres CLONE ID no. 1899078
MSSRRHVRYSPLAVDEDDDYHGGRRFDPRFDYSPKAFDRVPWKSIVLAVFLLCLGCLLLFLSFFIFSGHMGGEKSQAYGLLVLGILTFLPGFYETRIAYYSWRGAEGYSFASIPDY

SEQ ID NO: 217 - DNA, 721 bp, Panicum virgatum
  misc_feature (1)..(721): Ceres CLONE ID no. 1891899
agcaaagcgacggccccctcctccagttcccctccaacatactccctagcgaccagatcgagcttttccccggcgtggttaaggtcgttgtcctcggatcgacctcttgtccaaccaaatcgagcatcaatcgcttaaattagaaggtgatggtgctcagattgagcctcctgcgcattcgtccgcaccattttgcgtggatcctgtagcacgccttggttcttctccacgaactaactacacaggaacattctcagtgcagatactgaaatatggcatcaagacgcaatgttcgttacacacctcttcctgtagaggatggggagtacaacaatcctgctaaagaagatgttgatcttcggttcacttacactccaaaatcctacaggaggatcccctggaagtcaattgccctggcattgttcctcctcctccttggaacttcacttctcttcctttcatacttcatattcacaggtcacatggagggtgatagttctcaggcatatggtctcttattcctgggcttcctttccttccttcctggcttttacgaaactcgggttgcttactattcatggcgaggagcaccaggttacacctttgcatccataccagattattagcctctcagttctagccgcctttggtgtgcagtaaagcggcaagagagcagcctgtaacttcaactgggaaccaatttcagcgataaacatttagc

SEQ ID NO: 218 - protein, 117 aa, Panicum virgatum
  misc_feature (1)..(117): Ceres CLONE ID no. 1891899
MASRRNVRYTPLPVEDGEYNNPAKEDVDLRFTYTPKSYRRIPWKSIALALFLLLLGTSLLFLSYFIFTGHMEGDSSQAYGLLFLGFLSFLPGFYETRVAYYSWRGAPGYTFASIPDY

SEQ ID NO: 219 - DNA, 698 bp, Brassica napus
  misc_feature (1)..(698): Ceres CLONE ID no. 980825
aatccttatggtgaacagtctctctctctctctatccatcttatctctccttctgatctctctctttagatatcgttgatctctctctctctctcgtctctctcacctaaccttatcatttcacctgagactcgtccacgatccaggccatgagctccgaggaagctaaagtcgtcgtgccaaggaactttagattgttggaggagcttgagagaggcgagaaaggtatcggagatggtaccgtaagctatgggatggacgatgctgatgatatctatatgcaatcctggactggcaccatcctcggccctcataatactgcatacgaagggaaaatattccagctgaagctcttctgtggcaaggaataccctgaaagtccacctactgtgaggttccagacccggattaacatggcttgtgtcaaccccgaaactggagtggttgaaccgagtctcttccctatgctcgctaactggaggcgagaatacacaatggaggacattctgattaagctgaaaaaggaaatgatgacttcccataaccgcaagttagctcaacccccggaaggtactgaggaagctagggctgacctaaagggaccagctaaatgttgtgtgatgtgaagagagagaagttgaagggaatcgcatcaatttgtattaatagaagggataatgtatataaaacatcatttg

SEQ ID NO: 220 - protein, 158 aa, Brassica napus
  misc_feature (1)..(158): Ceres CLONE ID no. 980825
MSSEEAKVVVPRNFRLLEELERGEKGIGDGTVSYGMDDADDIYMQSWTGTILGPHNTAYEGKIFQLKLFCGKEYPESPPTVRFQTRINMACVNPETGVVEPSLFPMLANWRREYTMEDILIKLKKEMMTSHNRKLAQPPEGTEEARADLKGPAKCCVM

SEQ ID NO: 221 - DNA, 958 bp, Gossypium hirsutum
  misc_feature (1)..(958): Ceres CLONE ID no. 1850191
gttcctagtggactttgcgagatgtgaggtgtaggaggagttggactttcaggcactataaagtataaatctctctctctatctaatcagtacccttttacagttttactctttacaaatttccctctcctttttcgtgtttttcctcttaattttgctttttggttttggattgtctcttctgaatcagagaaaaccctaccttttttttctcttttatttctttgaattggaacgatcaaaagaaggaatgggttctgaaggatcgagggttgttgtgccaaggaatttcagattgcttgaagagcttgagagaggtgaaaagggaattggggatggaactgttagttatggaatggatgatgctgatgatatttacatgcagtcatggactgggactattattggcccacctaacaccgttcatgagggacgcatctaccagttgaaactattctgtggcctggattatccagataatccaccaagtgtgaggtttcaaacccggataaacatgacctgcgtcaatcaggaaactggagtggtcgaacctagccttttccccatgcttgctaattggcaaagggagtatacaatggaggatatattgactcagctgaagaaagaaatgatgtctccacagaataggaggatggctcagcctcctgaaggcaatgaagaagcaaggctcgatcaaaagggtttagtggtgaagtgttgcatcgtttgaagatcatcggagttgtatgaacataatgtatatacaatctatacgctagaaaaatccagcgaccagcttagcaccatcaatatatatatgcttcctctgtatgggaaagatgtccctggctccacctaatggtggttaaataaagcgaacttcttaatgaattgatgacatttgaaaatttatgacttgtgctcactgctggtatcgatgatatgttagttggcgagc

SEQ ID NO: 222 - protein, 159 aa, Gossypium hirsutum
  misc_feature (1)..(159): Ceres CLONE ID no. 1850191
MGSEGSRVVVPRNFRLLEELERGEKGIGDGTVSYGMDDADDIYMQSWTGTIIGPPNTVHEGRIYQLKLFCGLDYPDNPPSVRFQTRINMTCVNQETGVVEPSLFPMLANWQREYTMEDILTQLKKEMMSPQNRRMAQPPEGNEEARLDQKGLVVKCCIV

SEQ ID NO: 223 - DNA, 790 bp, Gossypium hirsutum
  misc_feature (1)..(790): Ceres CLONE ID no. 1838128
atagtggtagtggaccatcctgagttgtgatttgtgaaacagtagcttttgattttcctctcaactatttttgggctttcgcttttgaaatcagagaaaaaaaaccctaattcttaatctctttgttttgaaacgatcaagagaaggaatgggttctgaaggatcgagtgttgttgtgcctaggaatttcagattgctagaagaactcgagagaggtgaaaaaggaattggggatggaactgttagctatggaatggatgatgctgatgacgtatacatgcaatcgtggactggcactattatcggcccccctaatactgttcatgaaggacgcatctaccagttgaaattgttctgtggcatggattatcccgataacccaccgagcgtgaggtttcaaacccggataaatatgacctgtgtcaatcccgaaaccaaagtggttgaaccaagccttttccccatgcttgctaattggcgaagggagtatacaatggaggatatattgactcagctgaagaaagaaatgatatctccacaaaaccggaaacttactcagcctcctgaaggcaatgatgaggcgaggattgatcaaaagggtttagtggtgaagtgttgcatcttttaaacttaattgtatggaacataatgtacataatatatatatacacagataattcctacaactgccttagctcaatcatatatataatataatgcttgtgtatgagagagacgcccttggctccaccaaatggtggttaaataaatcgaacttcttaattaattt

SEQ ID NO: 224 - protein, 159 aa, Gossypium hirsutum
  misc_feature (1)..(159): Ceres CLONE ID no. 1838128
MGSEGSSVVVPRNFRLLEELERGEKGIGDGTVSYGMDDADDVYMQSWTGTIIGPPNTVHEGRIYQLKLFCGMDYPDNPPSVRFQTRINMTCVNPETKVVEPSLFPMLANWRREYTMEDILTQLKKEMISPQNRKLTQPPEGNDEARIDQKGLVVKCCIF

SEQ ID NO: 225 - DNA, 480 bp, Populus balsamifera subsp. trichocarpa
  misc_feature (1)..(480): Ceres ANNOT ID no. 1512371
atgggttctgaaggatcgagtgttgttgtgcctaggaacttcagactactggaggagcttgagagaggagaaaaggggatcggaaatggaactgtcagttatggaatggacgatgctgatgatatctacatgcagtcatggacaggaactataattggccccccaaatactgttcatgaagggcgtatctaccagttaaaattgttttgtggcaaggattatccagataatccgccgagtgtgaggttccaaactcggataaatatggcttgtgtcaatcctgcaagcggaatggtcgagcctagtcttttccctatgcttgctaattggcagagggagtgtacaatggaggatatattaactcagttgaagaaagaaatgatggctccacaaaacaggaagctcacccagcctcctgaaggaaatgaggaggcaaggttggatcaaaaggggctagtcctaaagtgttgtattctctga

SEQ ID NO: 226 - protein, 159 aa, Populus balsamifera subsp. trichocarpa
  misc_feature (1)..(159): Ceres ANNOT ID no. 1512371
MGSEGSSVVVPRNFRLLEELERGEKGIGNGTVSYGMDDADDIYMQSWTGTIIGPPNTVHEGRIYQLKLFCGKDYPDNPPSVRFQTRINMACVNPASGMVEPSLFPMLANWQRECTMEDILTQLKKEMMAPQNRKLTQPPEGNEEARLDQKGLVLKCCIL

SEQ ID NO: 227 - DNA, 873 bp, Panicum virgatum
  misc_feature (1)..(873): Ceres CLONE ID no. 1767492
aagaaatcgccgttgcagcaagtgaagctggaggaagcggaggggctcgcgatggggtccgagggaccgtcggctccggtcgtcgttcccagaaacttcaggctgctagaagaactagaacgtggtgagaagggcattggtgatgggacagtgagctacgggatggatgatgctgatgacatttatatgcggtcctggacaggaactatcattggccctcccaatactgttcacgagggacgaatctatcaattaaagcttttctgtgatacagattatccagataaaccaccaactgttcgatttcagactcgaatcaatatgacatgtgtgaacaaagaaactggattggttgaaccaagtctatttcctatgcttggaaactggcaaagggaacatacaatggaggacatcctaaccagcttaaaaagggagatgtcgactcctcagaatcgcaggctgcaccagcctcaagaaggtaatgaagaccaaagagtagagcagaaagggctgcctcatagatgtgtcatcatgtaacagatgtgcattgcaatgtacagcttgagttgagtagcatgactgcatgggatgcagaagtgaggaaccttctcaggaaatcgtgtaatttgtccaaactgcgcaagagttgcaataaagtatcgaggactctgttgatttgcctgcgcaccatgtaacatgttaccccattcaggttcagtgaaggacaatgagcttcacatcgctcattctgcctgtttgtaaacactctgtagtatgtaccgtcgggttatctgtctaggcctgttgttttatacaggaacagcgtattgataaatatcacagcctgattggttgttattgctctttctaccc

SEQ ID NO: 228 - protein, 161 aa, Panicum virgatum
  misc_feature (1)..(161): Ceres CLONE ID no. 1767492
MGSEGPSAPVVVPRNFRLLEELERGEKGIGDGTVSYGMDDADDIYMRSWTGTIIGPPNTVHEGRIYQLKLFCDTDYPDKPPTVRFQTRINMTCVNKETGLVEPSLFPMLGNWQREHTMEDILTSLKREMSTPQNRRLHQPQEGNEDQRVEQKGLPHRCVIM

SEQ ID NO: 229 - DNA, 953 bp, Gossypium hirsutum
  misc_feature (1)..(953): Ceres CLONE ID no. 1833556
tttatataaatatcaattacactaacctcaacaccgcagcacgttcatttcattatacaaaaaaagcagatagataggttgaactccctttttctccccttagaaccaaactttcaccgaccttcactttctgcctaccatgacggccaaggattgcggcaaccatggaaaaggccggcggcagcgcatccgaaggctcttcggttgccttctagtcttcctccttatcgttctcatcacaatccttatcatatgggcaatcctccgccccgcaaaaccccgcttcatcctccaagacaccaccgtctatgctttcaacgcctccactcccaatttcctcacttctaatttccaagtcaccgtatccacgcgaaaccccaatgacagaatcgggatttactacgacaggctcgtcatctacgccacctacaggaaccaacaaaccacccttaggacagctctccctccaacataccagggtcataatgagatcaatgtttggtcccctttcatctacgggaacatggtccctatagctcccgatttctccgtcgctttgaagtccgaacaagctgccggaacgatcttcatggtgatcaagattgatggacgggtgagatggaaagttgggactttcgtttcaggcaggtatcatctcaacgttaggtgtccggcttatatcactttcgggagcaaaaacaacggcgtttctgtcggagaaaacgctgttaagtaccagctcgtcaccagatgcagcgtcagtgtgtgagagagcactcatcaatatcaacaaagaagaagaccaggcaactaacgccgtttaatctatattaaatctctctctcagtttcttacttcacgccgttagtatattttatttattttctttataaggggaggaggaaattgtacattttgctatagaataaatgaaaaatggaaaattgaaagct

SEQ ID NO: 230 - protein, 209 aa, Gossypium hirsutum
  misc_feature (1)..(209): Ceres CLONE ID no. 1833556
MTAKDCGNHGKGRRQRIRRLFGCLLVFLLIVLITILIIWAILRPAKPRFILQDTTVYAFNASTPNFLTSNFQVTVSTRNPNDRIGIYYDRLVIYATYRNQQTTLRTALPPTYQGHNEINVWSPFIYGNMVPIAPDFSVALKSEQAAGTIFMVIKIDGRVRWKVGTFVSGRYHLNVRCPAYITFGSKNNGVSVGENAVKYQLVTRCSVSV

SEQ ID NO: 231 - DNA, 1153 bp, Panicum virgatum
  misc_feature (1)..(1153): Ceres CLONE ID no. 1816384
aatggacccataacgcccactttactcagcttcccaaacactgcctctgctctccctcttcttcctcattccctctccaccaccaccacattccaattccattccggccaccagagaagagaaacccaacaaagagccaccaccacccttcatcacttctcctccaccaaggcaccaaccagtccgggagccatgagcaaggactgcggcaaccacggcgacgacgacctccgccgctcgtgccggcgcctgctcggcctcctcctgggcctggccctcatcgtcgccatcatcgcgctcatcgtctacctggtcctccgccccacgcacccgcgcttcttcctgcaggacgcctcgctccgccagctcgacctcgccaacggctcctccaacctcctctccaccacgctccaggtcaccgtcgcctcccgcaaccccaacgaccgcgtcggcgtctactacgaccgcctcgacgtctacgcctcctacaagtaccagcagatcaccgtcgccgcctcgctcccgccggtctaccagggccacggcgacgtcgacgtctggtcgcccgtcctcgccggccccaacgtccccttcgccccctacctcgccgacgcgctccagcaggactgccaggccggctacctcatcctccaggtcaagatcgacggccgcgtcaggtggaaggtcggcagctggatctccggccactaccacctcttcgtcacctgcccggccttcctcgtcaccaacggcggcaacggcgcgccgggggccagcggattcaagttccagaccaccacctcctgccgcgtcgaggtctagctagctaccgaggctttggtgctgccgccggcgacggcgaggtaggtgcccgtacgtcgtcttgtgtctgtctgcctgtccagggttatagcagaaggggaaaaaacagaggccacagaagcagagagatcttgatgctcagggtcatggcggaaataatcgatcaattcatcaataccagtgaactgaatcaatcagtgattctgctgctgcgtataaaaatgtaaaggatgatgtttctttttttctttctttcgtagggttgattaataataatataatagtagtacgatcgatcgatggtcatatatatactatttatatatgctgc

SEQ ID NO: 232 - protein, 209 aa, Panicum virgatum
  misc_feature (1)..(209): Ceres CLONE ID no. 1816384
MSKDCGNHGDDDLRRSCRRLLGLLLGLALIVAIIALIVYLVLRPTHPRFFLQDASLRQLDLANGSSNLLSTTLQVTVASRNPNDRVGVYYDRLDVYASYKYQQITVAASLPPVYQGHGDVDVWSPVLAGPNVPFAPYLADALQQDCQAGYLILQVKIDGRVRWKVGSWISGHYHLFVTCPAFLVTNGGNGAPGASGFKFQTTTSCRVEV

SEQ ID NO: 233 - DNA, 1111 bp, Panicum virgatum
  misc_feature (1)..(1111): Ceres CLONE ID no. 1952828
agattcccaaaccgctgctctccctcttctacctcattccctctccaccaccaccacattccaattccattccggccaccagagaagagaaacccaacaaagagccaccaccactcttcatcacttctcctccaccaaagcaccaaccagtccgggagccatgagcaaggactgcggcaaccacggcgaccacgacctccgccgctcgtgccggcgcctgctcggcctcctcctgggcctggccctcatcgtcgccatcatcgcgctcatcgtctacctcgtcctccgccccacgcacccgcgcttcttcctgcaggacgcctcgctccgccagctagacctcgccaacggctcctccaagctcctctccaccacgctccaggtcaccgtcgcctcccgcaaccccaacgaccgcgtcggcgtctactacgaccgcctcgacgtctacgcctcctacaagtaccagcagatcaccgtcgccgcctcgctcccgccggtctaccagggccacggcgacgwcgacgtctggtcgcccgtcctcgccggccccaacgtccccttcgccccctacctcgccgacgcgctccagcaggactgccaggccggctacctcatcctccaggtcaagatcgacggccgcgtcaggtggaaggtcggcagctggatctccggccactaccacctcttcgtcacctgcccggccttccttgtcaccaacggcggcaacggcgcgccaggggcaagcggattcaagttccagaccaccacctcctgccgcgtcgaggtctagctagctaccgaggctttgctgctgccgccggcgacggcgaggtaggtgcccgtacgtcgtcttgtgtctgtctgcctgtccagggttatagcagaaggggaaaaaacagaggccacagaagcagagagatcttgatgctcagggtcatggcgataggcgaggcggaaataatcgatcaattcatcaataccagtgaactgaatcaatcagtgattctgctgctgcgtataaaaatgtaaaggatgatgtttctttttttctttctttcgtagggttgattaataataatataatagtaggacgatcgagcgagggtcacac

SEQ ID NO: 234 - protein, 209 aa, Panicum virgatum
  misc_feature (1)..(209): Ceres CLONE ID no. 1952828
MSKDCGNHGDHDLRRSCRRLLGLLLGLALIVAIIALIVYLVLRPTHPRFFLQDASLRQLDLANGSSKLLSTTLQVTVASRNPNDRVGVYYDRLDVYASYKYQQITVAASLPPVYQGHGDXDVWSPVLAGPNVPFAPYLADALQQDCQAGYLILQVKIDGRVRWKVGSWISGHYHLFVTCPAFLVTNGGNGAPGASGFKFQTTTSCRVEV

SEQ ID NO: 235 - DNA, 1104 bp, Populus balsamifera subsp. trichocarpa
  misc_feature (1)..(1104): Ceres ANNOT ID no. 1464039
atggatggaagctgcatagatgaaagcacaactagtagtgctgataactccatatccattacaccaacctccctccctcccttcccaccaactgctaccactacaaaatcaccacctgagtcactttgccgtgttaggagcggcaatagtagtgttattcttgattcggaatcaggtgtcgaagctgaatcgagaaaactcccttcttctaaatacaaaggtgttgtcccccaaccaaatggtcgctggggtgcacagatttacgagaagcaccagcgtgtgtggctcgggacgttcaatgaagaaaatgaagcagcaagagcctatgatattgctgctcagagattccgtggaagggatgccgtgactaacttcaagcaagttaatgagaccgaagatgatgaaatagaggctgctttcctgaacgctcattcgaaagctgaaatcgttgacatgttgaggaaacacacgtacagcgatgagctagagcaaagcaaaaggaaccacaggagtaacaatgggggaaatgggaagcaatacaagaatacagcaaactacgagaataatagttatgatcatggttgtggtcgggtgttgaaagcgcgtgaacagctttttgagaaagctgtgactccgagtgatgttgggaaattgaatcggcttgtgataccaaaacaacatgcggaaaagcattttcctttgcaaagtacatcaagcaatagtactaaaggtgtattgcttaacttggaagatgtgagcggcaaagtgtggaggtttcgttattcttattggaatagtagccaaagttatgttttgacaaaagggtggagccgatttgttaaagaaaagaacttgaaagctggtgacattgtttgctttcaaagatcaactggacctgataaccagctttacattgattggaaagcaagatgcgggtccaaccaggtccaaccggttcagatggtgaggttgtttggggttaatatttttaatgtacctgggatggaaaatgggtgtgatgggaagagatcaataagggatatggagcttttgtcaatagatcgtcagtatagtaagaaacaaaggatcgttggagccttgtaa

SEQ ID NO: 236 - protein, 367 aa, Populus balsamifera subsp. trichocarpa
  misc_feature (1)..(367): Ceres ANNOT ID no. 1464039
MDGSCIDESTTSSADNSISITPTSLPPFPPTATTTKSPPESLCRVRSGNSSVILDSESGVEAESRKLPSSKYKGVVPQPNGRWGAQIYEKHQRVWLGTFNEENEAARAYDIAAQRFRGRDAVTNFKQVNETEDDEIEAAFLNAHSKAEIVDMLRKHTYSDELEQSKRNHRSNNGGNGKQYKNTANYENNSYDHGCGRVLKAREQLFEKAVTPSDVGKLNRLVIPKQHAEKHFPLQSTSSNSTKGVLLNLEDVSGKVWRFRYSYWNSSQSYVLTKGWSRFVKEKNLKAGDIVCFQRSTGPDNQLYIDWKARCGSNQVQPVQMVRLFGVNIFNVPGMENGCDGKRSIRDMELLSIDRQYSKKQRIVGAL

SEQ ID NO: 237 - DNA, 1054 bp, Gossypium hirsutum
  misc_feature (1)..(1054): Ceres CLONE ID no. 1937560
aattaaggcggccttcattaaactcaaagaagaaacagaaaggctgaaaaagcatagcgaaggagagaagcaaaaacaaaaagaaatggattacctaggaatcgatcttagctgcgccattggatctctaaaagatggcaatttccctcccaaggactgtttgttgcctctcatctccaagctcctcggctacgccatcgtcgccgcttccaccaccgtcaaactccctcagatattgaaaattgtgaaccataaaagtgttagagggcttagtcttatagcctttgagcttgaagtagttggttataccattgctttagcgtattgccttcacaacggactacccttttcagcttatggggaattggtatttcttttgatccaagccctaatcttagttgctgttatttactactattcaaaacccgtgggcatcacgacctggatcagggcactgctatattgtgctgtagcaccaacaatcttagctggccaaattgatcctattctttttgaagctctatatgcatcccagcatgcaatatttttctttgccagggtgccacaaatttggaagaacttttctaacaaaagcactggggagctcagtttcttaacatgcttaatgaatgttgctggctcgcttgtgagagtatttaccagcctccaggaaaaagcaccaacaatggttcttttgggctctgtacttgggattgcaacaaatggcgcagtcctgagtcaaattattctctattggaattcacaagtgccgaaggagaagaaagcagagtaaattgataccatgtcagagtcagaagtaggaggaggttaagattggtgcagttggtgatcatcggatgtatgtattaccaagcaaatatcatcaacttatagttgacactatgaattggcaaatcttgtagatgattcattttagatattcgtaaatgggattcttctgcttttatgtgctttctttaggagttgatatgaatcacgctgcatttgcctgttggttagtattattttcattttatgttgaatatttgatatc

SEQ ID NO: 238 - protein, 235 aa, Gossypium hirsutum
  misc_feature (1)..(235): Ceres CLONE ID no. 1937560
MDYLGIDLSCAIGSLKDGNFPPKDCLLPLISKLLGYAIVAASTTVKLPQILKIVNHKSVRGLSLIAFELEVVGYTIALAYCLHNGLPFSAYGELVFLLIQALILVAVIYYYSKPVGITTWIRALLYCAVAPTILAGQIDPILFEALYASQHAIFFFARVPQIWKNFSNKSTGELSFLTCLMNVAGSLVRVFTSLQEKAPTMVLLGSVLGIATNGAVLSQIILYWNSQVPKEKKAE

SEQ ID NO: 239 - DNA, 637 bp, Zea mays
  misc_feature (1)..(637): Ceres CLONE ID no. 1448879
aaatcaaacaaaacatacatcgaccaaaaaaaggctaaagcttaattaacaaaacatacatcgatctcatggacaaggtgatgagaatgtcgtcagagaaaggagtggtgatcttcacgaagagctcatgttgtctctgctacgccgttcagatcctgttccgtgaccttagggttcaaccaacgatccacgagatcgacaacgatccagactgccgcgaaatcgagacggctctcctccgtatcggctgttccacggcggtgccagctgtcttcgtaggtggcaagctcgttggttccaccaatgaagtcatgtcacttcaccttagtggttctctcgtccccttgatcaagccctatcagtccctcctttaccaggccaattgaaccaactcgatcgagatccttcatataattaatcttaatattagctcgcttgtgacaataacaattcttatagctagagtgtgagctcgacgcatactagctactagatagaaattcggatgagttcttcttctgatattttatcatatgtatcttctgttctattgtttagcatctttcgtgtttcactctacttaatcaactggtgtatgttgatgtacgtgtataagtctctgtttattatgctaatg

SEQ ID NO: 240 - protein, 105 aa, Zea mays
  misc_feature (1)..(105): Ceres CLONE ID no. 1448879
MDKVMRMSSEKGVVIFTKSSCCLCYAVQILFRDLRVQPTIHEIDNDPDCREIETALLRIGCSTAVPAVFVGGKLVGSTNEVMSLHLSGSLVPLIKPYQSLLYQAN

SEQ ID NO: 241 - DNA, 592 bp, Zea mays
  misc_feature (1)..(592): Ceres CLONE ID no. 1490481
aaaacgcaaaccaaacaaacacatacatcgatcagacaataaaaaacttaactaaaccctagctagacatcagatctcatggacaaggtgatgagaatgtcgtcagaaagaggagtggtgatcatcacgaagagctcatgttgtctctgctacgccgttcagatcctgttccgtgaccttagggttcaaccaacggtccacgaaattgacaccgaccccgactgccgtgagatcgagaaggctcttctccggataggctgctccacggcggtaccagctgtcttcgtaggtggcaagctcgttggttccaccaatgaagtcatgtccctccaccttagtggctctctcgtccccttgatcaaaccttatcagtctctcctttactagctaactggaccaatcagagatccttcgtgacaataaaaacccttatagctatggtgtgcgcttttcttctgtcatatcacatatgtatcttatttttgtgtttcatgtttcagtctagttaatcaactggtgtatgttgatgtacgtataaatctctgtttatgattaccaaaaataaaaataaaaatctctgtttatgctaatg

SEQ ID NO: 242 - protein, 102 aa, Zea mays
  misc_feature (1)..(102): Ceres CLONE ID no. 1490481
MDKVMRMSSERGVVIITKSSCCLCYAVQILFRDLRVQPTVHEIDTDPDCREIEKALLRIGCSTAVPAVFVGGKLVGSTNEVMSLHLSGSLVPLIKPYQSLLY

SEQ ID NO: 243 - DNA, 653 bp, Gossypium hirsutum
  misc_feature (1)..(653): Ceres CLONE ID no. 1856294
gcaacaaccttaagctttgaacccttttctctcttttcccacttcaccttctcgaagtccaacctctccacaccgccattgcatcgtctgcactctcaactggatcccctagtcttttaagctttaagtgaaatcgacaaccaattgaaacattttcttcctcccctgttatcaaacattgtgaagcaggattttcaacatggacaaggtgacgagattggcttcagagaatgggttagtgctattcagcaagagctcatgttgcctgtgttatgcagtcaaaattctattccaagagattggggtgaccccaacggttcatgagctggaccaagaccccgaaggcagagaaatggagagggcgctcatgaggttggggtgcagcgccccagttccagccgtcttcattggtggaaagctggtgggttccaccaatgaagtcatgtccctccatctaagtggagggctaatacctttgctcaggccctatcactctatgtgttaaaatttaagcacgtcaattatatcagtaaaaaacagttgaataaaattggctcaaattaatcaaaggggggatgatcactttgaggagcggcatgtggattgtgtagtctgtgtagctttctatgtcttatgtgctttgtagctttcat

SEQ ID NO: 244 - protein, 101 aa, Gossypium hirsutum
  misc_feature (1)..(101): Ceres CLONE ID no. 1856294
MDKVTRLASENGLVLFSKSSCCLCYAVKILFQEIGVTPTVHELDQDPEGREMERALMRLGCSAPVPAVFIGGKLVGSTNEVMSLHLSGGLIPLLRPYHSMC

SEQ ID NO: 245 - DNA, 465 bp, Gossypium hirsutum
  misc_feature (1)..(465): Ceres CLONE ID no. 100028679
cgattcaatcggattcgaatgctgtgcttttaacatttaagtgaaatatctacaggtaatcaagggagtttctcctgttactaacagtgttcaatacagagaacattgtgaaccaggatttttcagcatggaaaaggtgacaagtttggcctcagagaaaggggtagtgctattcagcaagagctcatgttgcatgtgttatgcagtcaaaatcctattccaagagcttggggtaacccctatggttcatgagatagatcaagaccctgaaggctgggaaatggagaaagcactcaagaggttagggtgtaatggtcctgttccagccatcttcattggcgggaagctagtgggttccaccaatgaagttatgtccctccacctaagtggagggctcttacctatgctgaaaccttatcagactttgtgttaaataaaaaagtaaaacttcaaaaatatcacatt

SEQ ID NO: 246 - protein, 101 aa, Gossypium hirsutum
  misc_feature (1)..(101): Ceres CLONE ID no. 100028679
MEKVTSLASEKGVVLFSKSSCCMCYAVKILFQELGVTPMVHEIDQDPEGWEMEKALKRLGCNGPVPAIFIGGKLVGSTNEVMSLHLSGGLLPMLKPYQTLC

SEQ ID NO: 247 - DNA, 670 bp, Papaver somniferum
  misc_feature (1)..(670): Ceres CLONE ID no. 1629347
acatcacaatagcccagattctttctgtttcccgttgttctttacttgtatcttcatttaagttcaaaaacttctctaccatcggatcggaattcgacttcgaattcggtccgattggtttcgagattttacgtaaaagatgattaattagttctgtttttgtttctgttattgattgtgttaattgggtttctctattttgtcggatttcagaaacatatatactctatcagcttttctttggtcatattagcagttttaaattcagaaaaatggaagctataatgagggtggcatcaaagaaaggagttgtaatctttagtaagagttcgtgttgcatgtgttatgctgtcaagattctgtttcacgagctcggcgtgaacccgctgatccatgaacttgatcaagaccccgaaggaagagaaatggagaaagcacttgtgaggatgggatgtaatacacctgtaccatccgttttcattaatggtgagtttgtaggatccaccaatgaagttatgtctcttcacctcggaggttcattgcttccgttgcttagaccatatcaagctttaccttgatcagtataccagcactccgatctccatgtttgaagaataaccttggtttaatactacaaacccatattagccccttctttcgatatctatgt

SEQ ID NO: 248 - protein, 101 aa, Papaver somniferum
  misc_feature (1)..(101): Ceres CLONE ID no. 1629347
MEAIMRVASKKGVVIFSKSSCCMCYAVKILFHELGVNPLIHELDQDPEGREMEKALVRMGCNTPVPSVFINGEFVGSTNEVMSLHLGGSLLPLLRPYQALP

SEQ ID NO: 249 - DNA, 561 bp, Panicum virgatum
  misc_feature (1)..(561): Ceres CLONE ID no. 1768062
atgcaagctagcaaccaagcacaagtacccgaagaatcatacagcagatctcctatcctacattttcaaactgtccaagtttttcttttttcttttcccacaacaagacccacctgatcgatggaccgggtaacgaggctggcatcgcagcgtgctgtggtgatcttcaccacgagctcctgctgcatgtgtcacacagtgacgcagctcttccgtgagctcggggtgaacgcaacggtggtggagttggacggggaccctcggggaacggagatggacaaggcactggcgaggctgctgggccgcagcaccggtgtgccagcagtgttcatcggcggcaggctcgtcgggtccacggacaaggtcatgtcgctccacctcagcggcaacctcgtcccgctgctgcgcaatgcgggggcgctctgggtgtagcgtgatagggccttgtgctctatataagttagttttactgacagcaccatgtcgatccaatgcaaattatgggcttcagtattttgtaagagtacctgataaattgacgagtgagtgttttcctcccgg

SEQ ID NO: 250 - protein, 103 aa, Panicum virgatum
  misc_feature (1)..(103): Ceres CLONE ID no. 1768062
MDRVTRLASQRAVVIFTTSSCCMCHTVTQLFRELGVNATVVELDGDPRGTEMDKALARLLGRSTGVPAVFIGGRLVGSTDKVMSLHLSGNLVPLLRNAGALWV

SEQ ID NO: 251 - DNA, 909 bp, Populus balsamifera subsp. trichocarpa
  misc_feature (1)..(909): Ceres ANNOT ID no. 1459859
atggatttcttcctcaatgccaaaactgtccgtctccgcagccaccacgacaagtacctcctcgcggaggaagatgaggactccgtaacccaagaccgaaatggatcctccaagatagccagatggactgtcgaacccgtacctggatccgactccattatccgcctcaagtcctgcaatggtaaatacctcactgcctctaacgagccttttcttttgggcatgactggtcgaaaggtccttcagactgtacccagacgatttgactcgtccgtcgagtgggaacccgtgagagaagggggccaggtgaagctcaagacccggtatgggaacttcttgagagccaatggggggttgccaccttggagaaactcggttactcatgacattcctcataggagtgctacacaagattggatcctttgggatgttgatgttgtggagactcgagcgcttcagtctcctactggccatgctcattatctacagaaaattgtttctcagtctgattctctggattccgaatccacctctccaccttctatctctatcaaatccggagattatttaagacaggggtcgagtgattctaatgctagttcgcctcggaagtctgacgggaggacaatatactaccatgtggctgatgagagcggtgaggttgatgatgatgcaacagagcgttgctcgttgaattttaagggaaatggggtggatgggttgacacaaaaattgaaggaagatacgggacttgaggatattgttgtgtgtactcgcagtcctttgaatggagagctttatccacttcgattgcagcttcctccaaacaatgcagatatgcatgttattctagttcagccatcgtccaaagatgctgctgcatttaccgagagtccttgcatttcatga

SEQ ID NO: 252 - protein, 302 aa, Populus balsamifera subsp. trichocarpa
  misc_feature (1)..(302): Ceres ANNOT ID no. 1459859
MDFFLNAKTVRLRSHHDKYLLAEEDEDSVTQDRNGSSKIARWTVEPVPGSDSIIRLKSCNGKYLTASNEPFLLGMTGRKVLQTVPRRFDSSVEWEPVREGGQVKLKTRYGNFLRANGGLPPWRNSVTHDIPHRSATQDWILWDVDVVETRALQSPTGHAHYLQKIVSQSDSLDSESTSPPSISIKSGDYLRQGSSDSNASSPRKSDGRTIYYHVADESGEVDDDATERCSLNFKGNGVDGLTQKLKEDTGLEDIVVCTRSPLNGELYPLRLQLPPNNADMHVILVQPSSKDAAAFTESPCIS

SEQ ID NO: 253 - DNA, 406 bp, Gossypium hirsutum
  misc_feature (1)..(406): Ceres CLONE ID no. 100063116
tccttttctctcccgctctttgaatttccaaacttaaaaagctatgtctcagactgttgttctcaaggttggtatgtcatgtgagggctgcgttggagccgtgaagagagttttggggaaaatgcaaggtgtggaatcatatgaagtagatttgaaggagcagaaagtgacagtgaagggcaacgttcaacccgatgcggttctgcaaactgtgtcaaagactggaaagaagactgccttctgggaaggagaagctccggcagaagccgagacaaagccggccacggcttaaaatgtcttaagcccttattgattgattgattgattgattggtgtgcttataacatttcaaactatcctatgaactgtagaattcttggatgattgtgatttacttcctgtgttg

SEQ ID NO: 254 - protein, 82 aa, Gossypium hirsutum
  misc_feature (1)..(82): Ceres CLONE ID no. 100063116
MSQTVVLKVGMSCEGCVGAVKRVLGKMQGVESYEVDLKEQKVTVKGNVQPDAVLQTVSKTGKKTAFWEGEAPAEAETKPATA

SEQ ID NO: 255 - DNA, 614 bp, Panicum virgatum
  misc_feature (1)..(614): Ceres CLONE ID no. 1771295
aacggccaccccatctcccatcgcgtgtttcctgacgccaccgcctgtatttatctaccgccgttgcccatcgggagtcttccattccgtcgatcccgtcgagctccggatcccgcgtcccctgcgcgcctcgctctccggagagatctctacacttcttccctcgcgcgatggcccagactgttgtactcagggttggcatgtcctgtgaaggctgtgttggagctgttaagcgagttctgggcaaaatggaaggtgttgaattttacgatgtagacatcaaggagcagaaggtcacggtgaagggtaacgtaacacctgatgcagtactgcagactgtttccaagacaggcaaaaagacttcattctgggatgctgagcctgcaaccaaccaatccacagcacctgctgatgctactgcttgacaagttggatgtaacaaattccagtgtaattttctggtaaaactacaaatgtgtggggtcttatggtatttagcttcacaatattacaaataatgccagactgctattaaaatttgtacatcctgaatgcctgctattcctagcctgaggtttaaacagtttgggatgtaatggagaattgcttgtatg

SEQ ID NO: 256 - protein, 84 aa, Panicum virgatum
  misc_feature (1)..(84): Ceres CLONE ID no. 1771295
MAQTVVLRVGMSCEGCVGAVKRVLGKMEGVEFYDVDIKEQKVTVKGNVTPDAVLQTVSKTGKKTSFWDAEPATNQSTAPADATA

SEQ ID NO: 257 - DNA, 412 bp, Parthenium argentatum
  misc_feature (1)..(412): Ceres CLONE ID no. 1609456
aaatcaattcatttatcctctccccaaatcatatccctaatctcaaaatctataaccatgtctcagactgttgttcttaaggttggcatgtcgtgtggcggctgtgttggagccgtgaagagggttcttggcaaaatggaaggggttgaaacttttgacgttgatctggaacaacaaaaggtgactgtgaagggtaatgtacagccagatgcagttctgcagacagtttccaagaccgggaagaaaaccgagttctggccagctgaaggtgcatcagctactgcttgatctctgtgtactaaatggtttttgaagttggcaccccaattgtaaatctagaactatcaattgactgtaatattcagtattctgctcttgaataaatgcaaaacataattgttgtgttgtgatt

SEQ ID NO: 258 - protein, 76 aa, Parthenium argentatum
  misc_feature (1)..(76): Ceres CLONE ID no. 1609456
MSQTVVLKVGMSCGGCVGAVKRVLGKMEGVETFDVDLEQQKVTVKGNVQPDAVLQTVSKTGKKTEFWPAEGASATA

SEQ ID NO: 259 - DNA, 1836 bp, Oryza sativa
  misc_feature (1)..(1836): Ceres Promoter p530c10
gcctctcgaccacgagtttagcacttgtgcaacatatatgcgtgcgatgaacatctactgatgcgccatgcgaattttagcgttcgttcatgacgcttccaacggcacagaggctgagcagcagcatgcatgcatggctcttgtgaaaacaaaaaaggttactggtaaatgacatgctgctgtagctagctagcagaatgcaaggcccatgcatatgcaatgctatgcgacaagtacagtaccagcatgtatggtagccagctaactaatctatcagcagaggcagcaagctcgtgcatggtgtgatgcacttctctccagtaatctagtggtaattttcacccaaagcgttgctcatatggacagtaattagtaatattaccaaggttcacaatcccgttacctgaccaaatactactcacgaatggtatctctggttttcgttaaaaccgttggtaaaccagcaaaaatagacaaaatttgtcaaaattttaaattttagttttttttttttaacttagccgggaaaccttgaagtttgtgctgtcgagctgtcctgggaaggacggttttggttgggattgtgaaccctggttactgcacttcatttttgaacagatattagtgcaacagacaaatgccaacgcatttttttctgtttaccggcaagctgaagcttttacgatccccatacagccgttgctgcaaacctgccaagaaagagcagcagaaacaggtgtcattttgtggtggaaagccaagtaaagtaaacagaagatggaagatagtgaggaccagggagtgaggcaggggacacatggcccacgcctccctgcacattttcgtgtataaatacaggtggatgcatcgctctcccagcatccatcggttctctgctctgttcatccatagagtttcctcctcttctcctttagtgcaaggtagagaagagcatgtgtgtgtgtgtgtgtgtgtgaactgtgaagtgcagagtgcttctgtagttctgtgttatgtccatagtgatcttgttaggattgttgctatggatgcatgatgttatggttgatctctgaattacagtagggacttttctgagatctctggattagtggggggtgctaaatttttttctggttgcatcagcttgggtttctggtattggtgtgggttcttgctctgaattttggttcagaatgtcgatttgtttgtgtttgttctctgaagttgagagtagctatgatccatccagcacagaactgcaggtcctgcctgccggctgcatatacaggacatgccattttgcaagctctgggcttatggtttctcttttggagttcttcttcttgcatgatctgtgttctctaacaaaggaagcaagatttagcaactttattcagagacaagaaaaggatctggcaaccttttgtttctgttttatcctactcgtaaagattgttatttaagcaaaaatttcccaaaagttttaaatataatttccatgatgtgccactctcatgtccttgaacctggcactcattatgggctcctcagaagtgctgtagctaatgtcactaatcttttgtatctttgttcatagtcttgtattttatgatgcttatccctttgtgctttccatgtttgatgtccaaatgtcatggcaatgtttttgacttctagtaggggttttagtacctttttgttagataagtacatccaaattctgtttatttattcaaaaatcattctgtttattcactgaaaacatttgtccattcaatggactcataaactgtctgtgtttttcaggcttgaggatccatctagaagatagca

SEQ ID NO: 260 - DNA, 3000 bp, Oryza sativa
  misc_feature (1)..(3000): Ceres Promoter pOsFIE2-2
gcttaacacatgaactaccaaaatatactgatcactttgttctagtcatacataccttaagtcattttattctgcagtgtttggattggagggagcattctagcatcccttgggtcgttccagcaaatgtggttctccaaagcagagtaagcacaacacagtattttaggttatgtttcccctatctcgtcacggacagctcacaagttaatgtgatttatctcactatagatacgaagaacatggagtatcctacatccaaaggaagtgcccatgaagttgtggagcatcgctacgatttgtgaccaaatttgggtgcatgtgggcaatcgtattacagccaccctgttgttgatctatatcgactattatccgacgatatttatcattatattatgactagttagtttgtagattttgagagggcaacataagaagcaatccagcttaacctgttatgttcttgatggtagattctagttcatgtgttgaatctgttctccctgctgtagaatgtatcgagttgctgctctctactctgtacttttagaataccttttcaatcatttggagtcagctgattgttgtactacttatacgccacctgattagtcatgtcaacaattaaacttgagcactggttaagttaagagtggcctgattgtagttgataatcacattttattcgtagacattgtatgctggatctttatcagccaccgtcagatcatcctctgtaataaatcttcatcagacgtgtgtgccaatcgcaaggaacacgaaatgcatccgaaatgttactctgagttaatcaatactataattcttggtcaaattaattatttatatctataaagtttaaattaaatttaggaaaatgaattcatgcaaatcttgtggtaagttgtcaatttcataaaaaatccagcttactactccctttttaggagtgtgttgtggctgcacacttctgccttttgatatatacggttctattctcggtgtactcctttattattattaaaacaatcccagttacttggtaagtgctaatcacgaatcaaagtcaacataacaaatcatgtgcgtacagctataactcgattacacaaacaacaaaattcatatttgaacataaatccagttgtagcatatctggtagtataaagttttttttttgtatagaagagttttaatttctgtaagttttggaaagcatttaatcctagaaattgtagtgtagctcaactaaaaaataaatgaacttgaatcgaaattgggttgtatcataaatctttaccactcaaacgaatatttatcctaaaccacaaatgactcttttcatcaaggaatgttttgttttcagcattttaaaaaaaaacttttctaatatggttttcatgtttcgttcttttgaaatttaacatctatttaatttgcacggctccataaattcaacggatacatattctgaataattactaaggaggcatatatcggctctcttaatacaaccgcttgtttctcaaaatttattttgagttttgtctacacattctcaaggacggtacaaacacactatagatgttcacaattttttttttctaaagttgattgatggacaaatgtttgaacatataaacatataagcactgaatatttgcttatgcaggaggtatttatatcaagttcgatactttactaccatagtccctaggacactaaaatgccttcaatgatctgatgaagcctaagagagaatattgatcagtggagcgacttgcaactacacatggcacaagtagactagacacggtatatattcatattaacttgttaaaattttactacttaacagttcacttgtggtgcatccatatcaattcttacttacacaatatttgtaaaaacaacctaacactataggatgacctagacaacctttatgtcaatcacacttagaagatgatcgtctttttaataaataatgtgtactacacaccatgctctccatatagatcaagatctacaaacccttccacttataaaccttaccaccaaaaactcattaagttgcttcatttatctatgctattaagaaaaaaacttatttcgtttatgccatttctagaaatggctagtcacactattcacaatattatataataaataaaagtttcaaatattcatccaccaaaaatcatcaagtcgtgggacttatatgttaattagagaagtccctttgggtgcaatcgattttggaaaccctaaattttttctatacatagaagagagagatgtctagttgcaattgcttttgcgatgtgccaaccacccttctagctttcatccacgtctacttaattgccattcttcttcttctttttcttcactattactacctcctatcttagcgaatcttcttcttcttcactattactacctcccaccttagtgaattcatcctcattgttcacaatgacattgctaagttaactaggtatgctaagtacacaattagaatataacctagagcctttgtttccatcatacttaaaagatgacatttttatatagataaagtgtgctactcacaaggcttactatatatatgtatgatacacacaaactccacaacccaaaactctttcaagttgtgtggcccatctatgctattaaaaagcccatttagcccatccaacatgagaaaccctagggttttttccctataaaagatacctaggttattgttgcttttccaccccgcccgccgccgctccctattcctatttaatcccatctctcttcctcatcaccgctctcctctctccaggcaagaggtacgcactttttgtttcggatttgaaatctttgcttcgttttactatcattggtcataagttcttttttgaagatgtttgagaataagtttatcattgagattatcgtcacttgtgataggaagtacgcaacctcaagccggacaagacgtgagcaaag

SEQ ID NO: 261 - DNA, 2023 bp, Oryza sativa
  misc_feature (1)..(2023): Ceres Promoter pOsMEA
gagagcagaacatagtagccgctgttttctgggggtgcaatttgtgcaagatcgctatccttatggaccatgcaagcaccaagcaatattaagccaggtccaacagcggtcttggggaattcagaaatgagcttaaaaacctccttgagctggccagctcagccaaggaggtccatcatgcatgtgcatgctcaatacttggaattattgcaaaatgatcggtcattgactggaagactttgcgcccttcctcagccaaccttatgtggctgcatgcatagagtaccaacaggaaggtagcgtttgttggaataaggtttgcatccagcatgtccttgtagagcttcaaagcctcagcaccttggcccatgaaggccatatccagctaattgcattccatgagaccacattcttgctatccatactgttgaagtgaagatgctccgagcttcggaaatgcttccacactatgcatacatgtcaatgagcactgtcatgacataaacattgggccccaagtcctcctcagcgataatcctatgcagccactttcccagggacaaagctccaagctgtgcacacgctgaaagagagctagaaatgatgattggatttggtcacacgctaagtaccagcatttgctcaaagagggcaattgccatctccgtccagccattctaggcataccctggtattattgctttccatgattccgattccgtggtcttctatggcatcgcattgaaggccttccttgcagactccatatcatttaacctacagtacaatatggtaattgctgtcgacactggagaattcgcagtaaatccagacttgagaggaccatgtaagcattgatcaagcagttcattcccaaacagactatacgggatcagtgccagtgctcgagtttggcttcaattccaaggccatcaacccaataaacagattaactgatgaaccaaccatgcaattcgccgagcaaacatagattaagcattgtaggcaaccaaatctggattctccatcaagtcaaagagacgccatgcagaattccacatccccgctgtatacaccgagatcaaccggtcagaacatgctcatactccgccaaccctctcttcagaacatgctcatactccgccaaccctctcttctctgcaagaggcatcctccccaattccccattgttatatctgttgctggtaagaccgttgccagcgtggttgtgtcagaccgaacagactctgcactcgccatcctcacgaacgactccagggcctccgaaccaggaagcccggccggccatcagcgtgttccacataacggtatccggcgactgcacagtgtcgaacaccttgcgtgcgtggtcacctctggacagcatgaagcgtacaggctacagcttggccaatgcggacgccacgaacgtgtcggcggcgtaacccgcgcgtgcagcgcgccgcgcgcgggctgcggagtcggttggagacgacacgccgccgccatgagagcaatgagcgaggtggcggcgaaggcgaaggagaagtagtcgaggcaagcggaagagaaggcggcagcggagaaagcgatcggggcggcggaggaggtgggtgggagggagggacgcgtagcggaggtcggaggaggagggagctgaggtttccggggcgggggtcgagagggtagtgtacggaggcgagggacacggcgaggatctggtcgaggtagcgcagtgtgaaggaaagcgcgatgaggcggagggcgccggcgaagagcggcgcggcggatagcgggaggaggcggcgccggcggggtctcatccgattggaaacagattgggaagggggagggggtaggaatacgtggcgtcggcagtattaggtagagagagaaaccctttccatcctttgtctcttagccccgaaggagagagaaaaatcagaaaaaaaaaaccctccgcgtgtgggggaagcagagctccggacgctggcgccgctcgcgccaccgcacccgcaccgcc

SEQ ID NO: 262 - DNA, 2034 bp, Oryza sativa
  misc_feature (1)..(2034): Ceres Promoter pOsYp102
gaacgacccaaacgcgtaaatggtggtactggtttccctgctttgccgagtaccagcagccacgaagaacgttacacaatcgagtacaaaatctataagagcaagtttaatagcatagccaaatactacctctaaatcatctatagccaatttaatagttcatttattcaataattacttataaacatatactacaatcattaatatatggtcttacttcttatacacataatattttggagtccgtgttacagctggctataaatataagggattttggttggatgtggtacatcctattataatgaatctagacatgaaacctgtccaaattcatcgtgctaggatacgccacatctaaccaaaatctcttatctttagggatggagagagtaataattaaatgaagctaggtagagtttcccggtcaatacgcttgcgtgtgcttataagagcatggccaacagtttcccgatactcttcccaatatcagttttgaggagttttgttggaaaaaatcgctccaacagtagacctaaatcacccctaaaagcttggcgtttccaaacccgcatatttcgttctccacttgtagggaagagactcggcgcccaatccttcaaccgcatgcacttcgcgcgcgctgtgtgaaaattttcctaccaggttcttctttgtgcgttcgtctacctgtgagtcaatccatcacgccagcagcctcatcttccccgcagctgtctgggaaagcagccatggctcccccaagcttccccagcgtcgacatttttttctcagcggcagcgccagacccatctccaacccaattgggcggaccttcgtcggcgctcccccagcaccaccaccgactcgaatcggccgtcgcccctattcatctccaatcgtccctcgaccctaccgcatcctgcagcacagcctgtctctcgcgtcagactggcgctgcgctccccccggtaatgtgcaggcgacaaaggccccatgcgatgcgaccagcagccggcgacaaccggaggtgcccagtcgctggccttcatcgaatcatcgtgcacctcggtcggagtcgatttctgattgttgctgctgctcaaatctggagcttgctattgctgagaactgcttggtggtggtactggaaatttgttgtttgctggctgatgaaaactgttgttctttgctgctaaaaactgctgcttgctagtactgaaaagtactattgcagctgctgaaatatcttgctgcttgctgctgaaaacttcaagttgttaacaccgttcacactaaaaaagctgaaattttttttctgggctgaaaaccccattgttgatgattgcagaaccaatatttttccatgtaaaatacaggagatcgtggtaataatcaagtgaaatatcattttggggcaaatactcagatcgtacctgaagccaatggaaacattgttcaatgcttaaactgtcagttatgatgtcaaagagattgatcactgaatgtcctgaaaggagccgtgaggaggatgcagcattgcagcgtgcgcgagcgtgagtggaggagaggaatgacgattctgttggtagttgtcgatgtggcctactttttttgttttgaggattaaattttgggaatctcttggagataaaaggtattctcataccttaaatcctttttagagatctaaaaaaaatgatttaggggattgaattttgggtggctgttggtgatgctctaagttgcacatcctggggaaaaacctccctaatccatcagcaaaccgatcaaccacccacgacaagtcgacgccaccgttttttttttctccctcctaagtcctaaccccacaaaaatcccgcgaactttcgtctcaccacgcgccgcgtgccccctacaaataccaaacaacacccaccacgtccactcacaaaccacgcaggaaacctcagaaaatcaccgtacgcgacgcgggcccaagaaaaccccgacagaaaccgcgcagcagcaacaccaccaccggcgtcggag

SEQ ID NO: 263 - DNA, 1877 bp, Oryza sativa
  misc_feature (1)..(1877): Ceres Promoter pOsYp285
ggcccgagttaaacgatcttccacgtgtcagcgaatcctagtcgttcgatgaatctgaatctgacttgtggtggttggacggccacgtgttaaaaaagggaaacgtccgcatcacccgatgctgggacatttgcaatttcgatccagctgtagattgaccagttgttactctcttttttttaacaccatacaaacgtaatactccctctgtcccaaaatataagtattttttttaacctcggttcagtcttcgaggtgctactttgaccaataatatttataaaaataagatgttttaaataaagagagttgcatattatgatagctcgtttaatgataaacaaagtaccatcaaatttacatgattaatctttttaatttatttgctattaatagttaaaatttaaaaagtttgacttcacactgttctaaaaatacttatattttgggacggagggagtacacattagagcaggtacaatagcagactagtagccagctataaacatattttaatgagataaaagatgagagagaacagcgggctacagatctgtagccagctgcagcacggactccaagacattgtgtgtgtatgacaggtgggaccatatattaatagtacagtaagtaactattgtatgaattggctattagattagctataggtgaattgtagctagtagtgggctatactattgaacttactcttatatctctcaatatctccagaaaactaggacgatatatattgatattaacaaagtcatcatagatatctcgctatcgacatatatattacctatcactgaaaaaataattaatcataaatgcaagcacatatactacgttcaacactgaatgtaggtagattggtagacgggttccaccgcaagaaaagcattgcaccagtgaagaaagaaacatcggaatttgtatgtagtttgttgtttgatgaattcttttgattaaaaaaaactaaaatcagagttgattcagttaatggtgttgcctacgatatacttccatatcatgatatcactgtagactatgaatcatatctttaattaaaactaaatcaagaaattaagtatgagacctcaactcaatgaagaatttctagttgaaaaacattcctagtgtgcgttcggatggaggtagggatcttctctccgttcatataaaaccggatggttcattagaacatgattaattaagcaacagttaatctaaaaataaattaatattttttaagaaatttttgtatagagatcttttgaaaaaaatacattggttagaaagcatactaataaaaagagaaaaataagaacatagtactatagtagaaaatgagaacttggagtatttgagaggatgggaaataagaagattaagaagatgcgtaaagtgaacggttaacgcatgattgattaattaaatattaattattttaaatttggaaaataaattagtatgatttttaagcaacatatatatatatatatatatatatatagaaaaacatagttttagaaaatataagcgtgtaaaacgatatgcaggaacgaaacgttgagcattcaaaatttcaaattgaacatatgaatcaagagagaataaaaaaagaggccttctaggctggcatggacaattggacatgttttcaactagggtttcaagcttcgagcatccacttttgtccttgcaaactttatacggcaaggcccgtgaatctagccccccacaccaccccacccgcccgcgccgcgcggccgcctcgcctcccctcccttctcctcctctccgcccccgccgccaggccgtccacctccgccgtctcctcccccattcgcacccaaggcgctggcgcggaaggc

SEQ ID NO: 264 - DNA, 1000 bp, Arabidopsis thaliana
  misc_feature (1)..(1000): Ceres Promoter PT0565
caccaaatatagtgttatttcaatactaaaatggtgttatggttggagatgccctaaagataaacatgacgagacacgagatttattaatttcttgatcaaccataacttaataacttaatattaatttcacttaataatttccaattaagtgaatctttacttcaccaaaagttcctaacgaactcttattttctagcatcaatattaccatgaactagcatcaatactatcatgaaaaattcctacttcctatccaactcttaataacaatgctagtcttaacaatattcatcaaaaacttgatatagaccttctaacttagccacgactagtatcggtgaataccaaaattaatgtattcatgagaacttgagatttctctaatgtattcttgttactaaacaagtaacaacactcaagaaatatcatgatcaaatattttactcataaactccatatttcacattttgaaaattttaaacagcaaatcacattgaattttcgtggtaaaagtatttaaaattgaaaaatagcagctcctgatttcaatgtataaatttatctttatatggtttatgtctccaacttattttaaaaaagagagaaagagcacccaaaaggtgaccgtttgaaattcgaatttatttccgtttgaaattcgaattcaaaaaaagtaaaccgaaccgagtctcgttactgactgtcacacattgtttccctaaaagctaattaacccatacgtggcgtaatataacaggtcagtgatcaatactaaataacagacatacacctttaaaattcgtgcacgctccaaaacaaaatctacacttcaaaatcaacggtcacgatcattcctcaaatttcaaaaaattatttaacctcacttccttcgctttgtttttaaaacctctctctctttctctttctctttcgccattaaaactctgtttcctttttcagagattctcagagaagattcattttaccctaagaaaaaa

SEQ ID NO: 265 - DNA, 999 bp, Arabidopsis thaliana
  misc_feature (1)..(1000): Ceres Promoter YP0015
ttgagccttattgttgttattgacttttagccaatagaaagagatggaaattcaataattatccacaaaattccaaatcattggtgtacaaaaagatctaaggctgttatattttcaaaaaagaaagaaaagaaatgcaacaaatatggattaaactgtggtttgtaaattgagctttgcatgaaaactttatcactatgatttcactactccatatttattgactaaagtggcactaatgaatttcttaatcatgaaatcttgtatcaaaaagtactaaaataaacatgacattggcaattaggaaaattctaaattagaaattagtaaaaatgaaaggtgaaagggaaagatgatgatatgaattggttggtgaccaggagaaatgtatcccgatttttgcagacactttcagtgtccccattcatataattatggcccacctcgttaagatttttcattcaccaccataacaagatctaagcttagatttcatgtaattaaacatataatatacttgccaatactatctaataaagtatacttaagcaaaaattattactctagtgtaaggcgatgaaatataagtttagttgaaaatttatgtcgatataacaaagtataatgaattaagaccttggttttcgattaacaaactaattaaacactagttttgcctaataaaaccgggaatcgtattcaaaaccgaacgacaaaacaagggacaagttgagagacaaaaccaaatcagcatctttcttccagaaatgtcatgaccacatgacgtcatcttgacccttcttcattgtgatatctgtggataaagcgcacgtgtttaattcacgaaccttcgtagtaacgaaaaatccacaactttcatattttttaattacccactaaactaaaacaaatttggaaaaacatgaaaaactttttctttttttccaggttcgtgaacctcgtaccctctatataaacctcttaaccaccttccacata

SEQ ID NO: 266 - DNA, 999 bp, Arabidopsis thaliana
  misc_feature (1)..(1000): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 267 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 268 - DNA, 999 bp, Arabidopsis thaliana
  misc_feature (1)..(1000): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 269 - DNA, 999 bp, Arabidopsis thaliana
  misc_feature (1)..(1000): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 270 - DNA, 999 bp, Arabidopsis thaliana
  misc_feature (1)..(1000): Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 271 - DNA, 1000 bp, Arabidopsis thaliana
  misc_feature (1)..(1000): Ceres Promoter YP0388
agaagtattcacgcaccaaggttatatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaatagttttgacgatca

SEQ ID NO: 272 - DNA, 283 bp, Arabidopsis thaliana
  misc_feature (1)..(1000): Ceres Promoter PD0901
caaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatata

SEQ ID NO: 273 - DNA, 1000 bp, Arabidopsis thaliana
  misc_feature (1)..(1000): Ceres Promoter PT0623
aaagttattgacattttgaaaggaccgtaaatattaccaaaaaactgacggagttaggatcggccacgtagaaagggacaaagagagaacagtcacggactcggccagactaagtatgggcctgtctgaatccaaactcagctaagttccaaaagcataaagagagatgtgtaatgaaatgaacgtattctagaaacgaaagcaatgttatgctttgtttttgagccacatgtttttgggagatggagagaatcttttttacgtttttaacctaacccacttggcacttggccaaaaaagtgagaagaaactgtggcgaatgagtaggccacgccatggactttgttccttgtccttcaaaagttaaatttatgttatgcgtggggacaatctaagcaacgtggttcctttaaatatcgcagcttcctcttttacacttttggagcctacgtgttttgttttggaccggccaaatacacgagtcagtcagtttagaaataatttggatgtccaaaaatcttggagatccaaataaaataattagcatgttttagttcataagaatatgaaatgtagataaactgtctatattaatttttccatagaattggctttttatcgaggtgatgtacttaatgactttgttgattactactcgtataacaataaagaatatgatactatgtgagacttataatgaatttggtgtgtgttaattaatccagttgaaacagtttaataacaaatcagaataaaaattgtagtaagaaaatttgaacgctgatccttcaacctagatagtgaacctttcaaatactatatgattcacgtgtaatgtttttgaccgttggttatttttgtgtgaactatattaacttatcaatatcgaaaggctaaataagtaaataactaaaagaaagttcaggaaacaactcgacctaatgacctatcatttctgatcacccgtcctataaatacatacgtaagatcattcgttact

SEQ ID NO: 274 - DNA, 2709 bp, Populus balsamifera subsp. trichocarpa
  misc_feature (1)..(2709): Ceres ANNOT ID no. 1506868
atggcatcaaggcgcaatgttcgttacagttctcttcctgatgatgacaatgatgataaatttggtgtacaatatgaccctcgatttgattatacacctggatcttttgatagagtcccatggaagtccattttccttgcaatttttctgctcttccttggatgtgtacttctctctctgtcattctttatcttcactggtcacatgggaggagagaagtcccaagcttatggtctcctagctctgggaatcattaccttcatgccgggtatgttgaaactcaatgagcgattgattacataatatttacaacgaccatgatgaagactatgattagttaatcatttgtttatatctttggaaattttcacatgttccattctttatgttgcgagttaaatgtttctgtagaactaaggaacgaatggtgaaccaaaggttcttggctttttcgaggtttatcaggttaatgaataaaaaggaagtgcaaacagatttagaactaccttaaataacaaagtgatataatagaagttgatagttttttcaataaaatggttcactcacatgcaaggctagtttagactgttatttgttaaaaatgtgttgttagaggaagttttgatgattcatatttgctatataggcattgctcaacatgcctgactgtcattcaatgttttaattaacgtagttaatgccatgagtccaaaaggatgcgcactgaagtgatgctgtcttaggttgatcaaacttcctattgcttgtcattaagcataatgaatttcctctctttcgttttgtgcaaaatgattttcatgtagtgctcattattagtttgtatatgttagatcaaccttgtattgtctgtgacatctctctcatttttgtggtccacaaccttcagctcactgggatgtagaagttagttgtctaaaaaattatgaaagatattccaccatgtgtttatagaagcctgtaaaagcttgtggaacatgagggtgcactctctttattgcttctgtgatgctgatttaccaaaacggcacagcattgctgcaatttaggcttgcaattgaagaaataaaataagttaaagatgcggtaactggcccaaaaattttcagatcgctctccttgtgccatgatgttctagatcacatgtgaatagttgtggcgggggtggggtggggggtcaataaattagatttttagccttaaaaatagaaaattttaaattttggccccccatgaaatcttttctctgatttggccctcccaagctaaaattcctagctccgcccctgcggctacagtggaatcctacaaaccattaattacttgcaatttctccatgaagctttttgacatgatgatgtttgtatattgcttttgaattccttatttgaaatatggacaggatttgatggccaatcacttacctgcctttattgtattctgtaattaggatatggaaacttggtgtcaccttcgtttggtgaatttgtagagatattattgtgaatgagttctgctgattcagtttctacagacactgatcaagtttcctttagcttttatttgaatatatatataaattgctctaatagagcaatggtattctagacttcttgaaatgacaatactttaattgccttttttttgtgggggatgatggcatggttggggttggtgctcaataacaactaaataagcaccttttccatttggcttcctgccttttataccgcatagaaaattggttactatcatgatgctataggttctcctgttatggagtgaaactatattcatccttttcgattgctgaacttctgtaaaattaatactaaacgcattgagtatgtcttagaaccatctgactatagttcataagttcattaaatggaaggtttaaaattccacaatcaagaaaaaatgaaaaacccaatcaatattgctttgttattggttgttatggaggttaacgccttgcatttattgctgctaaatcacaccatattcatatgttgggtaaatccatatacatgtcgtagaagaaagcctatcctggccataaaaaacaggttgcttgtcttaaccgagacaaccaggatttagagtaggaagctagagcagatttagtcatgtccaaattcagaaatcatgatggctttgctagcattatgacaattaggtttatagaaagattggccacctaaattgcagaatgagcgtctaggatttgctcagtagatgtggaggatattgtgaaatggggactgacataaagtagtttaataatatggagttttcagttccttttcttttgaaacatagtaatttaacgacacatggaatttttattgagctatcatgaaatttataagcatggtgtttttgaggttgtttgtatccattatggactcgctttggcattgcctttttgtggaactcccatctgtgttcccacgttgcatttatttttcagctcataatccaaattacatgaaatcatatttccaattcaaccaatatgtttccatatgatttttctatgacatgctcttatatttttcatgacctgttcaactgaccactgctaaattgacctttgttgtggttgcaggcttttacgaaactcggatagcatattattcatggaggggtgctaagggatatcagtttgcttctatccctaaatattag

SEQ ID NO: 275 - DNA, 3129 bp, Populus balsamifera subsp. trichocarpa
  misc_feature (1)..(3129): Ceres ANNOT ID no. 1450498
atgtcgggcatgggagacgggtacgtgggcactgcccaagacgccgtgaggatccggcggctacagaagcaaagagaagctgaacgccttaaaatccaagagctcaaatctaagtcagcctccgctaaggaccagcccggtctcctccaattcgggtcaagtacatccgaggtaaatttatagggtttatctaaaaatctcattttctagggtttttaatcgtctgacctgattaattgctagttaactgcgattatgcctgtgttgatgttaatttggattcaatttgttttttagattctcgagactgcatttaagaaggaaactgtaggtctggttacaagagaacagtatgtagagaaggtagcgaagtctattaatttattgtttttgggtttcgtttttttgttaacgttagtgaaaatgataaggtgcttaatcaatatgttgtttgatgtgcgtttgtttacatagagggttaatattcagaccaaatttgaagaagaagagaaggagaagttagaaaagctacggaaagagtgagtttgattatggttttttcgttttatattttttgttttgattctgaatgttagtgaagatttttatcttggtaatggcagggaggaggagcttcagttgcagaagcggaaaaagaggaagattaaggggaattctaagttatcgtttgctgatgattttgagaatggaagtgatgaagaggatggcgaaaatagtaagaagttacattagttgacatactaagctatttaatctgtgtataagaatctggttttgtgttgggtgcattttgtggatattaccttttgctttgacgagagcattgtcaactagctaagcgctttatattctatttctgttttggaaacctgctgttaagctgtactacatcaaccatgtctgacttgttgtcgatggtcttattggtcttttggtgtccagttcgccttataatttcttgttagcttttttttatatataaaaaaaatatgttatttagattggctgaaaaggcttaagctgagttctcttcatgatcaaatcattctctgtacatatgttgaaagtggtttctcatggtttttaaattgtggcatggttagtatttttaggagagggaacatgaataataatattctggaataatagagtaggcactgttccttgtgcgtatccgagagtttcgaattgacttttctgtgcaggactcttgctttgtaaattcttccccgttgctatgaagtttgtttaactttatccaatcttggtagtgagaggaactggactacaacttttattttaacctaaacaattttctttatcaatgttatcttcagaaagcttagagccaaagagattagtgaacggtaaatttggaaaagatcccacagtggaaacaagctttttgcctgacaggtgcaatgtttttccttttccttccacctatcactctcccttcatgcaccttcaactgtgaaaggcttttgcatggacttggaactaacatatgagcttaggggtgctgaggatgctgtttcagtgagcgagaggcagaggagcaagctgagcgtgaaaggctgcggaaacagtggcttcgtgaacaggaacagattcgaagtaattattttcatgggcctctcctatttaacatggatgcaatgaaatacattttgagctaaatcaattttctctaatgactcagatgagccccttgaaatcacttacagctactgggatggagcaggccatagaagagtcatccaggttagacttcaccccacttttgtaatttattgacgtgctttcaatttttttcttatcaatattgtaaatagtttacatatcctttttcttattaattcatggttggtttattgctctctatctttcaggttcgtaaaggtgataccataggagagtttcttcgggcagttcagcaacaacttgcgccagagttcagagaaattagaacaacttctgtggagaatttgctttatgtgaaagaggatcttatcattccccatgtgagataattttgaaatccagcttttaataacaacagcttttgcacttgttatcttgaattcattcattctaacaaacttataccattttcatggctgtggaatttcagcaacacagtttctatgacctgatcattaacaaggctaggggaaaaagtggtccggtaagtctaatttcccaccctcttaactatccattgagtagccaacaagacaaaataagcatacatattatttgaaccatctaattagtgatattttccttcccttgtttttgtattcacttggacagtataagaattgaaaaactaagacaatggttggtttcgggacttgttcttcttataaaaaaatctatcatggaaagtgccgcaagtgcttaacctgttagtagtggcctacccaactacaattttcctgcaactctagattcttttactgagactgttggtgcttttaaaaaagcaattccagttctcagtataaattctcatgataatatgaaggtaatttgaatgattgtatttttattctttttctttgcacagcttttccactttgatgtgcacgaggatgtgcgaacaattgctgatgcaactatagagaaggatgaggtgtgttttttttataatctgtttgttgtttctacattgatctatggatttttctccattgtaacgtgcctatttgtgctttttcacatactttgctgaaacaaccatgcttattttttgtctttgttccttaacagtcccatgccggtaaagttgttgagaggcactggtatgaaaagaataaacacatctttcctgcttcaagatgggaggtgtgtcctccctctctctctgagcatattcacatatccgtatgcatgatttgtttccaaacacacgtttcacattttgtatcgtgtgtgtttcatggagcccggcagtacttgcatacctgctactaatgtggtggagccaaggggcagttttagtgatcagtttttacctcaagttcttataattttcctttgcgtttgtggcagatatatgacccgacacagaagtgggagcgttacaccatccatggggattga

SEQ ID NO: 276 - DNA, 3241 bp, Populus balsamifera subsp. trichocarpa
  misc_feature (1)..(3241): Ceres ANNOT ID no. 1460687
atgtcgggcatgggagacgggtacgtgggcacggcccaagacggcgtgaggatacggcggttacagaagcagagagaagctgaacgccttaaaatccaagagctcaaaaccaagtctgcctccgataacggccagcctggtctcctccaattcgggtcaagtacctccgaggtaaatttctagggtttcctgaaaaacaaatctcatttcttagggttttaatcatttctagctgaataattgttagttaacagcgatgatgtttgttttgatgattatctgggtttaatttgttcttttagattcttgagactgcatttaagaaggaaactgtgggtttggttacaagagaacagtacgtggagaaggtaataacgtttattattttcttttcttttccttttaatttcgctcttttgttaatgttaatgaaaatgttgagatgcttgagaaatgttgttgctgttgttgtgatgtgggtttatacagagggttaatattcagaccaagtttgaagaagaagagaaggagaaactagaaaagctacggaaagagtgagtttgattactgcgtttttgttttattttttgggttttgaatatgaatgtcagtgaagattgatatgtttgtattgccagggaggaggatcttcagttgcagaagcgtaaaaagaggaagattaaggggagttctaagttgtcttttgcggatgattttgagaatggaagtgacgaagaggatgttgaaaacagtaagaaatcacatgatttggcgtagtaagctatttactttgtgtattatctggatttgcgttggcttcatttttgggtaaaaaatgccttttgtttcgactagagcattgtccgctatcgaagtgctgcacattctaattctgtaatggacatttgcagtcaagttgtactgctgcatattctaattctgtaatgcacatttgcagtcaagttgtactgcatcggcctttctaactggttgttgattgtcctattattggtcttttagcatacagttcaccttatattttattggttgctgttaaggaatcaggttgtttagatgggttgaaaaggcttacattgaattttcatcatgatcaaatcacatgtgtatgtttgaagtgcttgctcctggcatttaaattgtggtcctgtgtttatttttagaaagagtgaacatgaataataatattctggagcagtagagtagccattgttttttatgtgtatgtgagaaatttggattgtcttatccatgcagaactcactttgtaattttttttgccctgctgctatgaactttgtttgattttacttgtatccttcttttcagtagtaataggaactgtactacaatttttacgttttaacctcaacacttcacttttatcaatgctgtctgcagaaacctcagagccaaataatttagtgcgggggaaatttggaaaagatcccactgtggaaacaagctttttgcctgacaggtgtaatatttttccctcaccctccttccctctttctacctctccctgtccctccatgcaccttctactttgaaaggcttttgcatggacttggaactaacatataacttaggggtgctgaggatgctgtttcagtgagcgagaggcagaggagcaggctgagcgtgaaaggctgcggaaacagtggcttcgtgaacaggaacagattcaaagtaattattttcctgaacctcttctatttaacatggatccattgaaacacactttgtgctaaatcatttttctctaatgtttcagatgagccccttgaaatcacttacagctactgggatggagcaggccatagaagagtgatccaggttagtttgacttcactttccttttcaatcttttgacatgctttcaatttccttttctcagtattggtaaaatgttttacttatcctttttcttattaattcatcattggcttgctgttttatcttttaggttcgtaaaggtgatgccataggagagtttcttcggggagttcagcaacaacttgcgccagagttcagagaaattaggactacttctgtggagaatttgctttatgtgaaagaggatcttatcattcctcatgtgagataattattaagtttaactttcaactacaatttttgcacttgctatcttgaattcatgctaacaaatttaaaacattttttgtggctgtggaatttcagcaacacagtttctatgagttgatcgttaacaaagctaggggaaaaagtggaccggtaaggctaatttcacactctctaactatccatcgagtagccaataagaaaaaaatgaagaattagacatggtgactatttagacaatctgattactgatattttccctgccttcttatttttgtattgacttgaaccgtatagaattgaaaaactaagttaatggttggttttggaaattgttcttttcattaacaaaaacctaacagggaaagtgctgcaagcacgttggtcctggcctacccaatcacatttttcttgcaaaccaagattcttttactaagattgttggtgctttcaaaaaagaactgccagttcttcttagtataatttctcataactggagagcttgagctaagtcgagcttgcgtctagtgttataaagtagtttatttcacgaacttaatttgaattattatgttttattcctttactttgaccagctcttccactttgacgtgcacgaggatgtgcgaacaattgctgatgcaaccatagagaaggatgaggtatgtctttcttgataccatctttttgttgtttctatattgatttccccctttataatgcgcctgattatgttgtctaaacatacttgcttaaactaccatgcttatttggtctttgttcttgaacagtcccatgctggtaaagttgttgagaggcactggtatgaaaagaacaaacacatctttcctgcttcgagatgggaggtgctctcttcatctatctctctctctatatttgtgcgcatgtgcacataacttgtatgcatgttttgtttccgcactcatgtttcacattctgtacggtgtttgtgggatagctgccactaatgtggtggagccagggtcaggtttaaggatcagttttcacctcaagttcttaccattttcgtttgtggttgtggcagatatatgatccgacaaagaagtgggagcgttacaccatccacggggattga

SEQ ID NO: 277 - DNA, 2101 bp, Populus balsamifera subsp. trichocarpa
  misc_feature (1)..(2101): Ceres ANNOT ID no. 1465436
atggaggtggtaattggaatttggtactgcaagaggggatgggtgtactatcaatatgtgtggtacaagtctgtacttgtactgccttcacttgaaagggaatccatacatatctcaacaaaaagacatggccttcttctcaagtttgctcagctaccgaatcaaaaccgtgcctccacaaaaatcttagattatgagacaaaaaaataaaaaaaaaatattaatactatcaatggaaaaaccattgccaataaaattatatttgttttttttttcttataaagtatatttgtcattgttgttgcttttacggttataatttttaaaaaatatttttaattatatttttaaaaattattttaaaaatatgcattagaattgttgtgagattgatttttttgcatataaaataaatcacagcatcatcacaaattggtaaaagaaacaacacaaatgttaattttttattgaaataatattaacaagttaaagacaaaataaaatattaaaacattgatccggaaatagaaatagttttatgttgactaacttaaaaaaaaaaaaggaaaaattgagattaaaaaaataggaaaatgttgccgggacccacaagaaaaacaaacaaaattcatcgcgcacataaaaaattctataaattcacgactttgaagaatgcataatcaagaacaaacttaaaatcttctctctgtcttcttgattccttgtttttcgttctgtttattacagcagcaataccataatcatgtctcaggtaccaaacattcttatttcttgattcatcttttatggcaaagtctatgtttttattgtgttatttacttgttgttttgacttgtgatgggatgtttatgagtctttgattgattttggattgccatgttgaatttcaagtttgaatcttttctagggttattgggtttttgttcattttgttttgtttgaggatttaagtgaaatctgttatggttaaaaaagttggtaaaggtgtcaaatttaattttgttaacctagtttttgagggagaaagattgctgctttgttgttggggagctgtggttttgaatcatttctggttttagttgaatgaaaattgtaggataaaagggatacaaaagtggctttttttctttttgtgttattgttacgagttagaaatttatctaggctcggatgttaactgaagtcttcacctgttgttgagcatgtcttggtttttgctgccttccctcttcttgatttgttgacattatgattgttgctgatttttgaaagtgtaatgattttcaattttgcttgatttcagactgttgtcctcaaggttggtatgtcatgcgaaggctgtgttggggctgtgaaaagggttttgggaaaaatggaaggtttgttcttgcccttgcttttgatctttttagtctctggagctttgttgaccgtgctgtctttctcccaacacacacacaaacaattgaaatggccaaacattttagatgggctttgctgggttgtcaatcttagtccttgatgctaaggtactattccaggaattttagggcaaggtgtacatcgtgttcatggtctatttgataatgaactagagcaaagattggttacagaattttgcacctttgcaagtaggttagttagcagtttggtatgatgaggtttcggagcatctgcaatttgtgaagattaggatatttactaattgtacattagaaagcaatgcacttctttactcttcatcaacgaataaagaaaaaaaaatgaaaggtagatttttgaagtattaatcaaatctggcttagaattccatgagcaaatgctattttcatatttagcggggacctttggttcatagtttctgttatttgatgataatagagtgtttcttctgtgagcatcttcaggtgtggaatcatatgacattgatttgaaggagcaaaaagtcacagtgaaaggaaatgtgcagccagatgctgttcttcagaccgtctctaagaccgggaagaagactgccttctgggaagcagaggcaccagctgaacccgcaaagcctgcagaaaccgtggctgctgcataa

SEQ ID NO: 278 - DNA, 4422 bp, Populus balsamifera subsp. trichocarpa
  misc_feature (1)..(4422): Ceres ANNOT ID no. 1512371
atgggttctgaaggatcgagtgttgttggtaagattaccctttcaaggaaaaaaaacctaactttccttgttagattttgtagtagtaatttttcttcttcttgttttttttcacgaggttagcattctgtgtttctttgcactttgcagtatctgtggtgtctttgagttggagttttgctccgaggtttacaattggttagggttttcgagttatcattgaggatgatctgtttttgagttgagattcaggatgatccgtttctcttagaccttttttttccctgattggctgtggtggaatggtttggaatttattgttttggctagatggttttctttacagcatctatatcttgtcctcgagcataaagaaattatcttgaagtaatttttttttgccttggagggctattttctccactcctgtacatgataaatgtttaatgaaatgcctcacaagatatgaaatgcatatgtggactttctgttacagagggagcaaatgccgagtacaatcaggctcctttgctcgagtttactcgtgcatctagagatcttacaaatcatgaccaagggtggcctttccaactctttggttttacctttagagagtaattttaaaatgggatgtgatacgtgggtaatggcagaaattagatggtatgagttggttgaattgaaagagatcagatttaagaggcctagaaacaattgatttttttgctgatttgggcaatgatgcaaattgtccctggagtggaaaaataaaatcctactcatgcgttgtaaatcaagtgagcttcctaattaaaagtagtatagatgaaataccacatttttgccccatgattttccatgtatctcccccaaatcctccaacagtaacaattttttctctctctgtttggtggtgtgtgtcttgggggggaggatttaatttctttttctatttgatgattcctagaatgcatgcatacatgattactattttgtaactgaatatttctgtcctcttcagtgcctaggaacttcagactactggaggagcttgagagaggagaaaaggggatcggaaatggaactgtcagttatggaatggacgatgctgatgatatctacatgcagtcatggacaggaactataattggccccccaaatgtatgtaatttttttgctattctctctttcaatattctcattcatgttttatgcatactggattgccaatgcatgaaccggttttaataaaattcttggtttaaattgcaacaaagtggtagattacgattcatgatgccagctcatttcattggtaagatgtctatgtttatggccttataggatgagaattcttgccttaatcctttaactatgctttgctggtgtgggctgtggaggcataaaagcagggtttttgaggagagaagttacttctctcagtcaatctggatatcgatttaaagtttccaagatgatattcatgcttatatgtgtgggttcttcaatcaaaaagtttaatgtatatgagttcacatgtttttttcttatcaattacactttctttcattgtcataatgagctattataggatacagactgcagttgactggcaaagcaactttctcggatttgaacatccatacgagcaagacaaaattttcaaggaataatttatctttcttacagttcatgtcatcagcccatctacttttaaagctttactactcatgaaaataaacatttgggaatatcattttctgaaaatattattggatgtcatttgaaacaattaaaatttcctcaacttcacagcaagtcagatacttatttttttcctctagaatcacaactgtcctttatcttttgatgacagatgttatttgactaacttaatgtatatatctgcaatctctttatatctcttctgcttccttcagctctcctattaaaaaaaactattttaacgttgttaaatcttggtgtcctttcagacgctctcaatcagaagtgtaatgtctttgtaatgcattagcaatccagttgcctttgcatattgttctgacttttttgtgaaatcttcaaggttctctttttgtttgtagtttgaagttttaaaatcatttctcttttatttctttttctgcattagatcgtcaatggaaaacttatttaacaaacaaacaaataaaagattggcactgcaaatcttatgttgtcaaatcttctataatctgataattcctcataaatcaagtagctttgatgccagtcttgccttgtcatctgcagaggttgattctgaaagaaaattgcagttgatctcttgtgctgtagtgtaccaacaatttttaaaacatggaaattgaaagagtataagggaaaggtgaaataaaatcactctaacatcagggaaagtagaaccaattctagatagtgacagcaaagaccttccaatgaaatctaaataataatttatccaaagaccttagaatatgcacttgccatgattctaaaataatattttagtcaaaataacttaaagataccccccagacaagaaaacccccaattgtcaaaacaaagaactacatcagatgaggtttggaatttctatttcccttagctacaccaaaaagattgacccctacttcacatgcacatatctatagtccaaatctgggcatctccatggcatttgatggtggcatcctatcatggtggataaggttggaatgagaatggtcctggaatggctatggtgctgctatttccaaacaccgcccccccccccccccccaagaaaaaaaatatcccagaaagcttctccatacctgctgcatacacaggtcaggggaaccaaagtcatccagagcctccaaggcccgcatcttacttgcttcatgaagcccttactgaaattagtgataaataaattcaaggtgaagttttttcttaggtagaattgcatcaattgctcatttgatctgaatgggcaaaaatgctagaagctttctaaaccctaaaatattgtaatagatcctaaaattttattgttaaaaaatttgcacgtctttttcaatgaatgcctggtagattctggttccaattaggttaggttgaattagtgggagagaattagtcacaattccctctaaatacagtgcataggtgattatgctggaaataagtacgtcttagaaatacgtgatcatctgggtggggtggcagggcttttttaaacttgctcctctaatgaatccaatcaaattatacatcttatcccattacccatccacggtattgccacaccttgtagtggcatggtaggcccccagtcctgactagtggggagaattgtcatccctagtttactttacaaagcagaccaccttatgactttgttggcctggcgtgatagtcatactatctctgccttgattcttgaatgatgagacatgatttttcaatgtaattatgtatgctttatattatttttcaaaaggaacaataatgtattgactcatgctacttgccagactgttcatgaagggcgtatctaccagttaaaattgttttgtggcaaggattatccagataatccgccgagtgtgaggttccaaactcggataaatatggcttgtgtcaatcctgcaagcggaatggtaggtgatgcaaattttagttattttacactaattgattgaaaatgatctcattggtaactaaattaacaggtcgagcctagtcttttccctatgcttgctaattggcagagggagtgtacaatggaggatatattaactcagttgaagaaagaaatgatggctccacaaaacaggaagctcacccagcctcctgaaggtgtgtctattttgcttctgcatattcttggtgtatcctatctatttggtattgattttctagtcaagcaagaataacttaatgaaattaacaactcttgttttcatggggagatgggaaggagtggttaaaaatgatgaaatgtgctgtttaggaagtgaaagatagacaccgccagtacacaaccaaggcaaaaacaaaattgaagtgtacatcagttaatgattgtactttgaatcacgggtgtgagtttgatttactgctcaaacgtttgaaatgattcaactaaattgggatttgttagcggttgttattaaatggagtgaaacccgatatgaactccatgcattagcactggcaccaagtcaggatggtaagatgcggaattatgagcaactgaacaatgggcatcttccttgcaatgcaattacttggtttatgcatatgttttgtaaaattctcaagaaatatacagtaatcattagttgtgtgtgtgcaggaaatgaggaggcaaggttggatcaaaaggggctagtcctaaagtgttgtattctctga

SEQ ID NO: 279 - DNA, 1218 bp, Populus balsamifera subsp. trichocarpa
  misc_feature (1)..(1218): Ceres ANNOT ID no. 1459859
atggatttcttcctcaatgccaaaactgtccgtctccgcagccaccacgacaagtacctcctcgcggaggaagatgaggactccgtaacccaagaccgaaatggatcctccaagatagccagatggactgtcgaacccgtacctggatccgactccattatccgcctcaagtcctgcaatggtaaatacctcactgcctctaacgagccttttcttttgggcatgactggtcgaaaggtccttcagactgtacccagacgatttgactcgtccgtcgagtgggaacccgtgagagaagggggccaggtgaagctcaagacccggtatgggaacttcttgagagccaatggggggttgccaccttggagaaactcggttactcatgacattcctcataggagtgctacacaagattggatcctttgggatgttgatgttgtggagactcgagcgcttcagtctcctactggccatgctcattatctacagaaaattgtttctcagtctgattctctggattccgaatccacctctccaccttctatctctatcaaatccggagattatttaagacagggggtaattctgatcttctcctgcttttcttaatgatttgattgaagtcctgaaatttcttttctttcttggtttttcatctgcttagtggtaatttgtttatggaatgttcagtcgagtgattctaatgctagttcgcctcggaagtctgacgggaggacaatatactaccatgtggctgatgagagcggtgaggttgatgatgatgcaacagagcgttgctcgttgaattttaagggaaatggggtggatgggttgacacaaaaattgaaggaagatacgggacttgaggatattgttgtgtgtactcgcagtcctttgaatggagagctttatccacttcgattgcagcttcctccaaacaatgcagatatgcatgttattctagttcagccatcgtccaaaggtgagcttataatggcttaagttcaatatattttgtttcgttctggtgttgctaatgcaaagcgtagttgttgaacttgtttgtcattttttgatgttgttctgatatacacaattcaatgctatgtgggttatagattattaacattgaaatctcagcttatcagaaatgaatgaggtgtattcattttatgtgtagatgctgctgcatttaccgagagtccttgcatttcatga

(19) United States
(12) Patent Application Publication
(10) Pub. No.: US 2009/0304901 A1
(43) Pub. Date: Dec. 10, 2009

(54) MODULATING PLANT PROTEIN LEVELS

(76) Inventors: Steven Craig Bobzin, Malibu, CA (US); Daniel Mumenthaler, Bonita, CA (US); Boris Jankowski, Newbury Park, CA (US); Joel Cruz Rarang, Granada Hills, CA (US)

Correspondence Address: FISH & RICHARDSON P.C., PO BOX 1022, MINNEAPOLIS, MN, 55440-1022, US

(21) Appl. No.: 12/161,928

(22) PCT Filed: Jan. 25, 2007
(86) PCT No.: PCT/US2007/061052

§ 371 Date: Apr. 16, 2009

Related U.S. Application Data
(60) Provisional application No. 60/762,226, filed on Jan. 25, 2006.

(51) Int. Cl. A23K 1/00; C12N 15/82; A01H 5/00; C12N 5/10; C07K 14/415; C07H 21/04

(52) U.S. Cl. 426/635; 800/278; 800/298; 435/419; 530/350; 536/23.6

(57) ABSTRACT

Methods and materials for modulating, e.g., increasing or decreasing, protein levels in plants are disclosed. For example, nucleic acids encoding protein-modulating polypeptides are disclosed as well as methods for using such nucleic acids to transform plant cells. Also disclosed are plants having increased protein levels and plant products produced from plants having increased protein levels.

### CROSS-REFERENCE TO RELATED APPLICATIONS

This Application claims priority under 35 U.S.C. 119(e) to U.S. Provisional Application No. 60/762,226, filed Jan. 25, 2006, incorporated herein by reference in its entirety.

### BACKGROUND

1. Technical Field

This document relates to methods and materials involved in modulating (e.g., increasing or decreasing) protein levels in plants. For example, this document provides plants having increased protein levels as well as materials and methods for making plants and plant products having increased protein levels.

2. Incorporation-By-Reference & Texts

The material in the accompanying sequence listing is hereby incorporated by reference into this application. The accompanying file, named 203WO1-Sequence.txt was created on Jan. 25, 2007 and is 470 KB. The file can be accessed using Microsoft Word on a computer that uses Windows OS.

3. Background Information

Protein is an important nutrient required for growth, maintenance, and repair of tissues. The building blocks of proteins are 20 amino acids that may be consumed from both plant and animal sources. Most microorganisms such as E. coli can synthesize the entire set of 20 amino acids, whereas human beings cannot make nine of them. The amino acids that must be supplied in the diet are called essential amino acids, whereas those that can be synthesized endogenously are termed nonessential amino acids. These designations refer to the needs of an organism under a particular set of conditions. For example, enough arginine is synthesized by the urea cycle to meet the needs of an adult, but perhaps not those of a growing child. A deficiency of even one amino acid results in a negative nitrogen balance. In this state, more protein is degraded than is synthesized, and so more nitrogen is excreted than is ingested.

According to U.S. government standards, the Recommended Daily Allowance (RDA) of protein is 0.8 gram per kilogram of ideal body weight for the adult human. The biological value of a dietary protein is determined by the amount and proportion of essential amino acids it provides. If the protein in a food supplies all of the essential amino acids, it is called a complete protein. If the protein in a food does not supply all of the essential amino acids, it is designated as an incomplete protein. Meat and other animal products are sources of complete proteins. However, a diet high in meat can lead to high cholesterol or other diseases, such as gout. Some plant sources of protein are considered to be partially complete because, although consumed alone they may not meet the requirements for essential amino acids, they can be combined to provide amounts and proportions of essential amino acids equivalent to those in proteins from animal sources. Soy protein is an exception because it is a complete protein. Soy protein products can be good substitutes for animal products because soybeans contain all of the amino acids essential to human nutrition and they have less fat, especially saturated fat, than animal-based foods. The U.S. Food and Drug Administration (FDA) determined that diets including four daily soy servings can reduce levels of low-density lipoproteins (LDLs), the cholesterol that builds up in blood vessels, by as much as 10 percent (Henkel, FDA Consumer, 34:3 (2000); fda.gov/fdac/features/2000/300_soy.html). FDA allows a health claim on food labels stating that a daily diet containing 25 grams of soy protein, that is also low in saturated fat and cholesterol, may reduce the risk of heart disease (Henkel, FDA Consumer, 34:3 (2000); fda.gov/fdac/features/2000/300_soy.html).

There is a need for methods of increasing protein production in plants, which provide healthier and more economical sources of protein than animal products.

### SUMMARY

This document provides methods and materials related to plants having modulated (e.g., increased or decreased) levels of protein. For example, this document provides transgenic plants and plant cells having increased levels of protein, nucleic acids used to generate transgenic plants and plant cells having increased levels of protein, and methods for making plants and plant cells having increased levels of protein. Such plants and plant cells can be grown to produce, for example, seeds having increased protein content. Seeds having increased protein levels may be useful to produce foodstuffs and animal feed having increased protein content, which may benefit both food producers and consumers.

In one aspect, a method of modulating the level of protein in a plant is provided. The method comprises introducing into a plant cell an isolated nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, and the consensus sequences set forth in FIGS. 1-9, where a tissue of a plant produced from the plant cell has a difference in the level of protein as compared to the corresponding level in tissue of a control plant that does not comprise the nucleic acid.

In another aspect, a method of modulating the level of protein in a plant is provided. Thc method comprises introducing into a plant cell an isolated nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, and the consensus sequences set forth in FIGS. 1-9, where a tissue of a plant produced from the plant cell has a difference in the level of protein as compared to thc corresponding level in tissue of a control plant that does not comprise the nucleic acid.

In another aspect, a method of modulating the level of protein in a plant is provided. The method comprises introducing into a plant cell an isolated nucleic acid comprising a nucleotide sequence encoding a polypeptide having 80 percent or greater sequence identity to an amino acid sequence selected. from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:11, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, and SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, where a tissue of a plant produced from the plant cell has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:81. The nucleotide sequence can encode a polypeptide comprising an amino acid sequence corresponding to SEQ ID NO:83. The nucleotide sequence can encode a polypeptide comprising an amino acid sequence corresponding to SEQ ID NO:95. The nucleotide sequence can encode a polypeptide comprising an amino acid sequence corresponding to SEQ ID NO:107. The nucleotide sequence can encode a polypeptide comprising an amino acid sequence corresponding to SEQ 5 ID NO:114. The nucleotide sequence can encode a polypeptide comprising an amino acid sequence corresponding to SEQ ID NO:119. The nucleotide sequence can encode a polypeptide comprising an amino acid sequence corresponding to SEQ ID NO:127. The nucleotide sequence can encode a polypeptide comprising an amino acid sequence corresponding to SEQ ID NO:148. The nucleotide sequence can encode a polypeptide comprising an amino acid sequence corresponding to SEQ ID NO:155. The nucleotide sequence can encode a polypeptide comprising an amino acid sequence corresponding to SEQ ID NO:167. The nucleotide sequence can encode a polypeptide comprising an amino acid sequence corresponding to a consensus sequence set forth in FIG. 1, FIG. 2, FIG. 3, FIG. 4, FIG. 5, FIG. 6, FIG. 7, FIG. 8, or FIG. 9. The difference can be an increase in the level of protein. The isolated nucleic acid can be operably linked to a regulatory region. The regulatory region can be a tissue-preferential regulatory region. The tissue-preferential regulatory region can be a promoter. The regulatory region can be a broadly expressing promoter. The plant can be a dicot. The plant can be a member of the genus Arachis, Brassica, Carthamus, Glycine, Gossypium, Helianthus, Lactuca, Linum, Lycopersicon, Medicago, Olea, Pisuln, Solanum, Trifolium, or Vitis. The plant can be a monocot. The plant can be a member of the genus Avena, Elaeis, Hordeum, Musa, Oryza, Panicum, Phleum, Secale, Sorghum, Triticosecale, Triticum, or Zea. The tissue can be seed tissue.

A method of producing a plant tissue is also provided. The method comprises growing a plant cell comprising an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, and the consensus sequences set forth in FIGS. 1-9, where the tissue has a difference in the level of protein as compared to the corresponding level in tissue of a control plant that does not comprise the nucleic acid.

In another aspect, a method of producing a plant tissue is provided. Thc method comprises growing a plant cell comprising an exogenous nucleic acid. comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, and the consensus sequences set forth in FIGS. 1-9, where the tissue has a difference in thc level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, and SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, where the tissue has a difference in the level of protein as compared to the corresponding level in tissue of a control plant that does not comprise the nucleic acid.

The sequence identity can be 85 percent or greater. The sequence identity can be 90 percent or greater. The sequence identity can be 95 percent or greater. The nucleotide sequence can encode a polypeptide comprising an amino acid sequence corresponding to SEQ ID NO:81. The nucleotide sequence can encode a polypeptide comprising an amino acid sequence corresponding to SEQ ID NO:83. The nucleotide sequence can encode a polypeptide comprising an amino acid sequence corresponding to SEQ ID NO:95. The nucleotide sequence can encode a polypeptide comprising an amino acid sequence corresponding to SEQ ID NO:107. The nucleotide sequence can encode a polypeptide comprising an amino acid sequence corresponding to SEQ ID NO:114. The nucleotide sequence can encode a polypeptide comprising an amino acid sequence corresponding to SEQ ID NO:119. The nucleotide sequence can encode a polypeptide comprising an amino acid sequence corresponding to SEQ ID NO:127. Thc nucleotide sequence can encode a polypeptide comprising an amino acid sequence corresponding to SEQ ID NO:148. The nucleotide sequence can encode a polypeptide comprising an amino acid sequence corresponding to SEQ ID NO:155. The nucleotide sequence can encode a polypeptide comprising an amino acid sequence corresponding to SEQ ID NO:167. The nucleotide sequence can encode a polypeptide comprising an amino acid sequence corresponding to a consensus sequence set forth in FIG. 1, FIG. 2, FIG. 3, FIG. 4, FIG. 5, FIG. 6, FIG. 7, FIG. 8, or FIG. 9. The difference can be an increase in the level of protein. The exogenous nucleic acid can be operably linked to a regulatory region. The regulatory region can be a tissue-preferential regulatory region. The tissue-preferential regulatory region can be a promoter. The regulatory region can be a broadly expressing promoter. The plant tissue can be dicotyledonous. The plant tissue can be a member of the genus Arachis, Brassica, Carthamus, Glycine, Gossypium, Helianthus, Lactuca, Linum, Lycopersicon, Medicago, Olea, Pisum, Solanum, Trifolium, or Vitis. The plant tissue can be monocotyledonous. The plant tissue can be a member of the genus Avena, Elaeis, Hordeum, Musa, Oryza, Panicum, Phleum, Secale, Sorghum, Triticosecale, Triticum, or Zea. The tissue can bc seed tissue.

A plant cell is also provided. The plant cell comprises an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO: , SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, and the consensus sequences set forth in FIGS. 1-9, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, and thc consensus sequences set forth in FIGS. 1-9, where a tissue of a plant produced. from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, and SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, SEQ ID NO:228, where a tissue of a plant produced from the plant cell has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:81. The nucleotide sequence can encode a polypeptide comprising an amino acid sequence corresponding to SEQ ID NO:83. The nucleotide sequence can encode a polypeptide comprising an amino acid sequence corresponding to SEQ ID NO:95. The nucleotide sequence can encode a polypeptide comprising an amino acid sequence corresponding to SEQ ID NO:107. The nucleotide sequence can encode a polypeptide comprising an amino acid sequence corresponding to SEQ ID NO:114. The nucleotide sequence can encode a polypeptide comprising an amino acid sequence corresponding to SEQ ID NO:119. The nucleotide sequence can encode a polypeptide comprising an amino acid sequence corresponding to SEQ ID NO:127. The nucleotide sequence can encode a polypeptide comprising an amino acid sequence corresponding to SEQ ID NO:148. The nucleotide sequence can encode a polypeptide comprising an amino acid sequence corresponding to SEQ ID NO:155. The nucleotide sequence can encode a polypeptide comprising an amino acid sequence corresponding to SEQ ID NO:167. The nucleotide sequence can encode a polypeptide comprising an amino acid sequence corresponding to a consensus sequence set forth in FIG. 1, FIG. 2, FIG. 3, FIG. 4, FIG. 5, FIG. 6, FIG. 7, FIG. 8, or FIG. 9. The difference can be an increase in the level of protein. The exogenous nucleic acid can be operably linked to a regulatory region. The regulatory region can be a tissue-preferential regulatory region. The tissue-preferential regulatory region can be a promoter. The regulatory region can be a broadly expressing promoter. The plant can be a dicot. The plant can be a member of the genus Arachis, Brassica, Carthamus, Glycine, Gossypium, Helianthus, Lactuca, Linum, Lycopersicon, Medicago, Olea, Pisum, Solanum, Trifolium, or Vitis. The plant can be a monocot. The plant can be a member of the genus Avena, Elaeis, Hordeum, Musa, Oryza, Panicum, Phleum, Secale, Sorghum, Triticosecale, Triticum, or Zea. The tissue can be seed tissue.

A transgenic plant is also provided. The transgenic plant comprises any of the plant cells described above. Progeny of the transgenic plant are also provided. The progeny have a difference in the level of protein as compared to the level of protein in a corresponding control plant that does not comprise the exogenous nucleic acid. Seed and vegetative tissue from the transgenic plant are also provided. In addition, food products and feed products comprising seed or vegetative tissue from the transgenic plant are provided. Protein from the transgenic plant, which can be soybean, is also provided.

In another aspect, an isolated nucleic acid molecule is provided. The isolated nucleic acid molecule comprises a nucleotide sequence having 95% or greater sequence identity to the nucleotide sequence set forth in SEQ ID NO:105.

In another aspect, an isolated nucleic acid molecule is provided. The isolated nucleic acid molecule comprises a nucleotide sequence having 95% or greater sequence identity to the nucleotide sequence set forth in SEQ ID NO:87.

In another aspect, an isolated nucleic acid is provided. The isolated nucleic acid comprises a nucleotide sequence encoding a polypeptide having 80% or greater sequence identity to the amino acid sequence set forth in SEQ ID NO:88.

In another aspect, an isolated nucleic acid molecule is provided. The isolated nucleic acid molecule comprises a nucleotide sequence having 95% or greater sequence identity to the nucleotide sequence set forth in SEQ ID NO:98.

In another aspect, an isolated nucleic acid is provided. The isolated nucleic acid comprises a nucleotide sequence encoding a polypeptide having 80% or greater sequence identity to the amino acid sequence set forth in SEQ ID NO:99.

In another aspect, an isolated nucleic acid molecule is provided. The isolated nucleic acid molecule comprises a nucleotide sequence having 95% or greater sequence identity to the nucleotide sequence set forth in SEQ ID NO:120.

In another aspect, an isolated nucleic acid is provided. The isolated nucleic acid comprises a nucleotide sequence encoding a polypeptide having 80% or greater sequence identity to the amino acid sequence set forth in SEQ ID NO:121.

In another aspect, an isolated nucleic acid molecule is provided. The isolated nucleic acid molecule comprises a nucleotide sequence having 95% or greater sequence identity to the nucleotide sequence set forth in SEQ ID NO:122.

In another aspect, an isolated nucleic acid is provided. The isolated nucleic acid comprises a nucleotide sequence encoding a polypeptide having 80% or greater sequence identity to the amino acid sequence set forth in SEQ ID NO:123.

In another aspect, an isolated nucleic acid molecule is provided. The isolated nucleic acid molecule comprises a nucleotide sequence having 95% or greater sequence identity to the nucleotide sequence set forth in SEQ ID NO:140.

In another aspect, an isolated nucleic acid is provided. The isolated nucleic acid comprises a nucleotide sequence encoding a polypeptide having 80% or greater sequence identity to the amino acid sequence set forth in SEQ ID NO:141.

In another aspect, an isolated nucleic acid. molecule is provided. The isolated nucleic acid molecule comprises a nucleotide sequence having 95% or greater sequence identity to the nucleotide sequence set forth in SEQ ID NO:142.

In another aspect, an isolated. nucleic acid is provided. The isolated nucleic acid comprises a nucleotide sequence encoding a polypeptide having 80% or greater sequence identity to the amino acid sequence set forth in SEQ ID NO:143.

In another aspect, an isolated nucleic acid molecule is provided. The isolated nucleic acid molecule comprises a nucleotide sequence having 95% or greater sequence identity to the nucleotide sequence set forth in SEQ ID NO:159.

In another aspect, an isolated nucleic acid is provided. The isolated nucleic acid comprises a nucleotide sequence encoding a polypeptide having 80% or greater sequence identity to the amino acid sequence set forth in SEQ ID NO:160.

In another aspect, an isolated nucleic acid molecule is provided. The isolated nucleic acid molecule comprises a nucleotide sequence having 95% or greater sequence identity to the nucleotide sequence set forth in SEQ ID NO:215.

In another aspect, an isolated nucleic acid is provided. The isolated nucleic acid. comprises a nucleotide sequence encoding a polypeptide having 80% or greater sequence identity to the amino acid sequence set forth in SEQ ID NO:216.

In another aspect, an isolated nucleic acid molecule is provided. The isolated nucleic acid molecule comprises a nucleotide sequence having 95% or greater sequence identity to the nucleotide sequence set forth in SEQ ID NO:217.

In another aspect, an isolated nucleic acid is provided. The isolated nucleic acid comprises a nucleotide sequence encoding a polypeptide having 80% or greater sequence identity to the amino acid sequence set forth in SEQ ID NO:218.

In another aspect, an isolated nucleic acid molecule is provided. The isolated nucleic acid molecule comprises a nucleotide sequence having 95% or greater sequence identity to the nucleotide sequence set forth in SEQ ID NO:221.

In another aspect, an isolated nucleic acid is provided. The isolated nucleic acid comprises a nucleotide sequence encoding a polypeptide having 80% or greater sequence identity to the amino acid sequence set forth in SEQ ID NO:222.

In another aspect, an isolated nucleic acid molecule is provided. The isolated nucleic acid molecule comprises a nucleotide sequence having 95% or greater sequence identity to the nucleotide sequence set forth in SEQ ID NO:223.

In another aspect, an isolated nucleic acid is provided. The isolated nucleic acid comprises a nucleotide sequence encoding a polypeptide having 80% or greater sequence identity to the amino acid sequence set forth in SEQ ID NO:224.

In another aspect, an isolated nucleic acid molecule is provided. The isolated nucleic acid molecule comprises a nucleotide sequence having 95% or greater sequence identity to the nucleotide sequence set forth in SEQ ID NO:225.

In another aspect, an isolated nucleic acid is provided. The isolated nucleic acid comprises a nucleotide sequence encoding a polypeptide having 80% or greater sequence identity to the amino acid sequence set forth in SEQ ID NO:226.

In another aspect, an isolated nucleic acid molecule is provided. The isolated nucleic acid molecule comprises a nucleotide sequence having 95% or greater sequence identity to the nucleotide sequence set forth in SEQ ID NO:227.

In another aspect, an isolated nucleic acid is provided. The isolated nucleic acid comprises a nucleotide sequence encoding a polypeptide having 80% or greater sequence identity to the amino acid sequence set forth in SEQ ID NO:228.

In another aspect, an isolated nucleic acid molecule is provided. The isolated nucleic acid molecule comprises a nucleotide sequence having 95% or greater sequence identity to the nucleotide sequence set forth in SEQ ID NO:229.

In another aspect, an isolated nucleic acid is provided. The isolated nucleic acid comprises a nucleotide sequence encoding a polypeptide having 80% or greater sequence identity to the amino acid sequence set forth in SEQ ID NO:230.

In another aspect, an isolated nucleic acid molecule is provided. The isolated nucleic acid molecule comprises a nucleotide sequence having 95% or greater sequence identity to the nucleotide sequence set forth in SEQ ID NO:231.

In another aspect, an isolated nucleic acid is provided. The isolated nucleic acid comprises a nucleotide sequence encoding a polypeptide having 80% or greater sequence identity to the amino acid sequence set forth in SEQ ID NO:232.

In another aspect, an isolated nucleic acid molecule is provided. The isolated nucleic acid molecule comprises a nucleotide sequence having 95% or greater sequence identity to the nucleotide sequence set forth in SEQ ID NO:233.

In another aspect, an isolated nucleic acid is provided. The isolated nucleic acid comprises a nucleotide sequence encoding a polypeptide having 80% or greater sequence identity to the amino acid sequence set forth in SEQ ID NO:234.

In another aspect, an isolated nucleic acid molecule is provided. The isolated nucleic acid molecule comprises a nucleotide sequence having 95% or greater sequence identity to the nucleotide sequence set forth in SEQ ID NO:235.

In another aspect, an isolated nucleic acid is provided. The isolated nucleic acid comprises a nucleotide sequence encoding a polypeptide having 80% or greater sequence identity to the amino acid sequence set forth in SEQ ID NO:236.

In another aspect, an isolated nucleic acid molecule is provided. The isolated nucleic acid molecule comprises a nucleotide sequence having 95% or greater sequence identity to the nucleotide sequence set forth in SEQ ID NO:237.

In another aspect, an isolated nucleic acid is provided. The isolated nucleic acid. comprises a nucleotide sequence encoding a polypeptide having 80% or greater sequence identity to the amino acid sequence set forth in SEQ ID NO:238.

In another aspect, an isolated nucleic acid molecule is provided. The isolated. nucleic acid molecule comprises a nucleotide sequence having 95% or greater sequence identity to the nucleotide sequence set forth in SEQ ID NO:243.

In another aspect, an isolated nucleic acid is provided. The isolated nucleic acid comprises a nucleotide sequence encoding a polypeptide having 80% or greater sequence identity to the amino acid sequence set forth in SEQ ID NO:244.

In another aspect, an isolated nucleic acid molecule is provided. The isolated nucleic acid molecule comprises a nucleotide sequence having 95% or greater sequence identity to the nucleotide sequence set forth in SEQ ID NO:245.

In another aspect, an isolated nucleic acid is provided. The isolated nucleic acid comprises a nucleotide sequence encoding a polypeptide having 80% or greater sequence identity to the amino acid sequence set forth in SEQ ID NO:246.

In another aspect, an isolated nucleic acid molecule is provided. The isolated nucleic acid molecule comprises a nucleotide sequence having 95% or greater sequence identity to the nucleotide sequence set forth in SEQ ID NO:249.

In another aspect, an isolated nucleic acid is provided. The isolated nucleic acid comprises a nucleotide sequence encoding a polypeptide having 80% or greater sequence identity to the amino acid sequence set forth in SEQ ID NO:250.

In another aspect, an isolated nucleic acid molecule is provided. The isolated nucleic acid molecule comprises a nucleotide sequence having 95% or greater sequence identity to the nucleotide sequence set forth in SEQ ID NO:251.

In another aspect, an isolated nucleic acid is provided. The isolated nucleic acid comprises a nucleotide sequence encoding a polypeptide having 80% or greater sequence identity to the amino acid sequence set forth in SEQ ID NO:252.

In another aspect, an isolated nucleic acid molecule is provided. The isolated nucleic acid molecule comprises a nucleotide sequence having 95% or greater sequence identity to the nucleotide sequence set forth in SEQ ID NO:253.

In another aspect, an isolated nucleic acid is provided. The isolated nucleic acid comprises a nucleotide sequence encoding a polypeptide having 80% or greater sequence identity to the amino acid sequence set forth in SEQ ID NO:254.

In another aspect, an isolated nucleic acid molecule is provided. The isolated nucleic acid molecule comprises a nucleotide sequence having 95% or greater sequence identity to the nucleotide sequence set forth in SEQ ID NO:255.

In another aspect, an isolated nucleic acid is provided. The isolated nucleic acid comprises a nucleotide sequence encoding a polypeptide having 80% or greater sequence identity to the amino acid sequence set forth in SEQ ID NO:256.

In another aspect, an isolated nucleic acid molecule is provided. The isolated nucleic acid molecule comprises a nucleotide sequence having 95% or greater sequence identity to the nucleotide sequence set forth in SEQ ID NO:274.

In another aspect, an isolated nucleic acid molecule is provided. The isolated nucleic acid molecule comprises a nucleotide sequence having 95% or greater sequence identity to the nucleotide sequence set forth in SEQ ID NO:275.

In another aspect, an isolated nucleic acid molecule is provided. The isolated nucleic acid molecule comprises a nucleotide sequence having 95% or greater sequence identity to the nucleotide sequence set forth in SEQ ID NO:276.

In another aspect, an isolated nucleic acid molecule is provided. The isolated nucleic acid molecule comprises a nucleotide sequence having 95% or greater sequence identity to the nucleotide sequence set forth in SEQ ID NO:277.

In another aspect, an isolated nucleic acid molecule is provided. The isolated nucleic acid molecule comprises a nucleotide sequence having 95% or greater sequence identity to the nucleotide sequence set forth in SEQ ID NO:278.

In another aspect, an isolated nucleic acid molecule is provided. The isolated nucleic acid. molecule comprises a nucleotide sequence having 95% or greater sequence identity to the nucleotide sequence set forth in SEQ ID NO:279.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of Lead 121-Ceres Clone 11852 (SEQ ID NO:83) with homologous and/or orthologous amino acid sequences Ceres Clone:975428 (SEQ ID NO:84), Ceres Clone:635196 (SEQ ID NO:86), Ceres Annot:1506868 (SEQ ID NO:88), Ceres Clone:891349 (SEQ ID NO:89), Ceres Clone:1602143 (SEQ ID NO:91), and gi|77548568 (SEQ ID NO:92). The consensus sequence determined by the alignment is set forth.

FIG. 2 is an alignment of Lead 122-Ceres Clone 8166 (SEQ ID NO:95) with homologous and/or orthologous amino acid sequences Ceres Clone:1064651 (SEQ ID NO:96), Ceres Clone:970655 (SEQ ID NO:97), Ceres Annot:1475146 (SEQ ID NO:99), Ceres Clone:465057 (SEQ ID NO:100), gi|62701864 (SEQ ID NO:103), and Ceres Clone:632710 (SEQ ID NO:104). The consensus sequence determined by thc alignment is set forth.

FIG. 3 is an alignment of Lead 123-Ceres Clone 38311 (SEQ ID NO:107) with homologous and/or orthologous amino acid sequences gi|72140114 (SEQ ID NO:109), gi|33320073 (SEQ ID NO:110), and gi|34895690 (SEQ ID NO:112). The consensus sequence determined by the alignment is set forth.

FIG. 4 is an alignment of Ceres Clone 109289 (SEQ ID NO:114) with homologous and/or orthologous amino acid sequences Ceres Clone:566154 (SEQ ID NO:115) and Ceres Clone:218121 (SEQ ID NO:117). The consensus sequence determined by the alignment is set forth.

FIG. 5 is an alignment of Ceres Clone 19342 (SEQ ID NO:119) with homologous and/or orthologous amino acid sequences Ceres Annot:1450498 (SEQ ID NO:121), Ceres Clone:1043576 (SEQ ID NO:124), and gi|50726581 (SEQ ID NO:125).

FIG. 6 is an alignment of Ceres Clone 21006 (SEQ ID NO:127) with homologous and/or orthologous amino acid sequences Ceres Clone: 1079973 (SEQ ID NO:128), Ceres Clone:1030898 (SEQ ID NO:131), Ceres Clone:510704 (SEQ ID NO:139), Ceres Annot:1525141 (SEQ ID NO:141), gi|53748489 (SEQ ID NO:144), and gi|58737210 (SEQ ID NO:145).

FIG. 7 is an alignment of Ceres Clone 2296 (SEQ ID NO:148) with homologous and/or orthologous amino acid sequences Ceres Clone:525163 (SEQ ID NO:149), gi|50937115 (SEQ ID NO:150), Ceres Clone:242812 (SEQ ID NO:151), and Ceres Clone:687022 (SEQ ID NO:153).

FIG. 8 is an alignment of Ceres Clone 33038 (SEQ ID NO:155) with homologous and/or orthologous amino acid sequences Ceres Clone:1064435 (SEQ ID NO:157), Ceres Clone:622673 (SEQ ID NO:158), Ceres Annot:1465436 (SEQ ID NO:160), gi|30039180 (SEQ ID NO:162), Ceres Clone:625242 (SEQ ID NO:163), and gi|50942155 (SEQ ID NO:165).

FIG. 9 is an alignment of Ceres Clone 5821 (SEQ ID NO:167) with homologous and/or orthologous amino acid sequences gi|71040677 (SEQ ID NO:170), Ceres Clone:540991 (SEQ ID NO:171), gi|50918253 (SEQ ID NO:172), Ceres Clone:616699 (SEQ ID NO:173), and Ceres Clone:220463 (SEQ ID NO:175).

### DETAILED DESCRIPTION

The invention features methods and materials related to modulating (e.g., increasing or decreasing) protein levels in plants. In some embodiments, the plants may also have modulated levels of oil. The methods can include transforming a plant cell with a nucleic acid encoding a protein-modulating polypeptide, wherein expression of the polypeptide results in a modulated level of protein. Plant cells produced using such methods can be grown to produce plants having an increased or decreased protein content. Such plants, and the seeds of such plants, may be used to produce, for example, foodstuffs and animal feed having an increased protein content and nutritional value.

### Polypeptides

The term “polypeptide” as used herein refers to a compound of two or more subunit amino acids, amino acid analogs, or other peptidomimeties, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include protein-modulating polypeptides. Protein-modulating polypeptides can be effective to modulate protein levels when expressed in a plant or plant cell. Modulation of the level of protein can be either an increase or a decrease in the level of protein relative to the corresponding level in control plants.

A protein-modulating polypeptide can be a polypeptide that is involved in plant defense responses, such as a harpin-induced family polypeptide. A protein-modulating polypeptide can also be a nuclear polypeptide, such as a transcription factor polypeptide, or a membrane bound polypeptide. A protein-modulating polypeptide can also be an electron carrier polypeptide or a polypeptide that transports heavy metals. A protein-modulating polypeptide can also be an enzyme, such as an ubiquitin-conjugating enzyme. A protein-modulating polypeptide can also be a polypeptide of unknown function.

A protein-modulating polypeptide can be a harpin-induced family polypeptide. Harpin-induced family polypeptides are reported to be up-regulated during the hypersensitive response generated by an incompatible plant-pathogen interaction and during senescence. SEQ ID NO:95 sets forth the amino acid sequence of an Arabidopsis clone, identified herein as Ceres Clone 8166 (SEQ ID NO:94), that is predicted to encode a harpin-induced family polypeptide. A protein-modulating polypeptide can comprise the amino acid sequence set forth in SEQ ID NO:95. Alternatively, a protein-modulating polypeptide can be a homolog, ortholog, or variant of the polypeptide having the amino acid sequence set forth in SEQ ID NO:95. For example, a protein-modulating polypeptide can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:95.

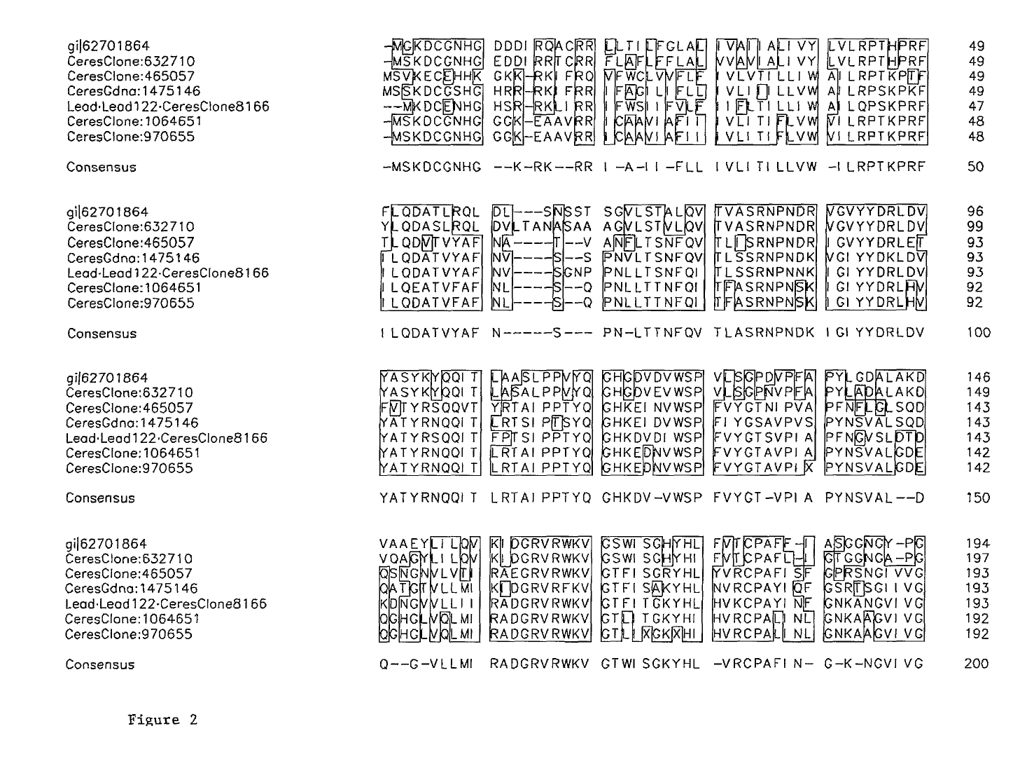

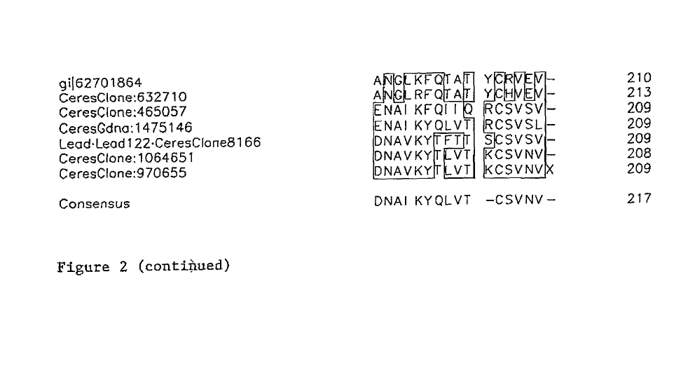

Amino acid sequences of homologs and/or orthologs of the polypeptide having the amino acid sequence set forth in SEQ ID NO:95 are provided in FIG. 2, along with a consensus sequence. A consensus amino acid sequence for such homologs and/or orthologs was determined by aligning amino acid sequences, e.g., amino acid sequences related to SEQ ID NO:95, from a variety of species and determining the most common amino acid or type of amino acid at each position. For example, the alignment in FIG. 2 provides the amino acid sequences of Ceres Clone 8166 (SEQ ID NO:95), Ceres Clone:1064651 (SEQ ID NO:96), Ceres Clone:970655 (SEQ ID NO:97), Ceres Annot:1475146 (SEQ ID NO:99), Ceres Clone:465057 (SEQ ID NO:100), gi|62701864 (SEQ ID NO:103), and Ceres Clone:632710 (SEQ ID NO:104). Other homologs and/or orthologs include Ceres CLONE ID no. 650444 (SEQ ID NO:101), Ceres Clone:662698 (SEQ ID NO:102), Public GI no. 77553726 (SEQ ID NO:105), Ceres Clone:1833556 (SEQ ID NO:230), Ceres Clone:1816384 (SEQ ID NO:232), and Ceres Clone:1952828 (SEQ ID NO:234).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:96, SEQ ID NO:97, SEQ ID NO:99, SEQ ID NO:100, SEQ ID NO:101, SEQ ID NO:102, SEQ ID NO:103, SEQ ID NO:104, SEQ ID NO:105, SEQ ID NO:230, SEQ ID NO:232, SEQ ID NO:234 or the consensus sequence set forth in FIG. 2.

SEQ ID NO:81 sets forth the amino acid sequence of an Arabidopsis clone, identified herein as Ceres Clone 120446 (SEQ ID NO:80), that is predicted to encode a polypeptide of unknown function. A protein-modulating polypeptide can comprise the amino acid sequence set forth in SEQ ID NO:81. Alternatively, a protein-modulating polypeptide can be a homolog, ortholog, or variant of the polypeptide having the amino acid sequence set forth in SEQ ID NO:81. For example, a protein-modulating polypeptide can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:81.

A protein-modulating polypeptide can have a DUF872 domain characteristic of a eukaryotic polypeptide of unknown function. SEQ ID NO:83 sets forth the amino acid sequence of an Arabidopsis clone, identified herein as Ceres Clone 11852 (SEQ ID NO:82), that is predicted to encode a eukaryotic polypeptide of unknown function. A protein-modulating polypeptide can comprise the amino acid sequence set forth in SEQ ID NO:83. Alternatively, a protein-modulating polypeptide can be a homolog, ortholog, or variant of the polypeptide having the amino acid sequence set forth in SEQ ID NO:83. For example, a protein-modulating polypeptide can have an amino acid sequence with at least 55% sequence identity, e.g., 55%, 60%, 65%, 70%, 75%, 80%, 85%, 90%, 95%, 97%, 98%, or 99% sequence identity, to the amino acid sequence set forth in SEQ ID NO:83.

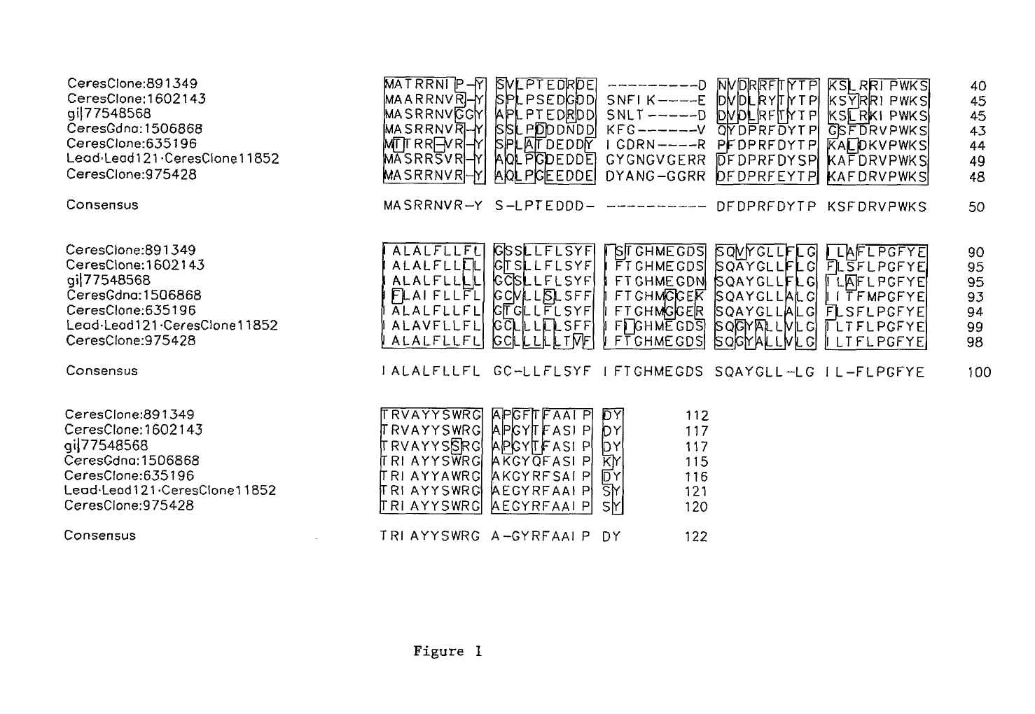

Amino acid sequences of homologs and/or orthologs of the polypeptide having the amino acid sequence set forth in SEQ ID NO:83 are provided in FIG. 1, along with a consensus sequence. A consensus amino acid sequence for such homologs and/or orthologs was determined by aligning amino acid sequences, e.g., amino acid sequences related to SEQ ID NO:83, from a variety of species and determining the most common amino acid or type of amino acid at each position. For example, the alignment in FIG. 1 provides the amino acid sequences of Ceres Clone 11852 (SEQ ID NO:83), Ceres Clone:975428 (SEQ ID NO:84), Ceres Clone:635196 (SEQ ID NO:86), Ceres Annot:1506868 (SEQ ID NO:88), Ceres Clone:891349 (SEQ ID NO:89), Ceres Clone: 1602143 (SEQ ID NO:91), and gi|77548568 (SEQ ID NO:92). Other homologs and/or orthologs include Ceres CLONE ID no. 965227 (SEQ ID NO:85), Ceres Clone: 1054465 (SEQ ID NO:90), Public GI no. 77553579 (SEQ ID NO:93), Ceres Clone:1899078 (SEQ ID NO:216), and Ceres Clone:1891899 (SEQ ID NO:218).

In some cases, a protein-modulating polypeptide includes a polypeptide having at least 80% sequence identity, e.g., 80%, 85%, 90%, 95%, 97%, 98%, or 99% sequence identity, to an amino acid. sequence corresponding to SEQ ID NO:84, SEQ ID NO:85, SEQ ID NO:86, SEQ ID NO:88, SEQ ID NO:89, SEQ ID NO:90, SEQ ID NO:91, SEQ ID NO:92, SEQ ID NO:93, SEQ ID NO:216, SEQ ID NO:218, or the consensus sequence set forth in FIG. 1.

A protein-modulating polypeptide can be a transcription factor polypeptide containing B3 and AP2 domains. A B3 DNA binding domain is found in VP1/AB13 transcription factor polypeptides, which have various roles in development. Some polypeptides having a B3 domain also have a second, AP2 DNA binding domain. AP2 is a prototypic member of a family of transcription factors unique to plants, which has the distinguishing characteristic that all members contain the so-called AP2 DNA-binding domain. SEQ ID NO:107 sets forth the amino acid sequence of an Arabidopsis clone, identified herein as Ceres Clone 38311 (SEQ ID NO:106), that is predicted to encode a transcription factor polypeptide containing B3 and AP2 domains. A protein-modulating polypeptide can comprise the amino acid sequence set forth in SEQ ID NO:107. Alternatively, a protein-modulating polypeptide can be a homolog, ortholog, or variant of the polypeptide having the amino acid sequence set forth in SEQ ID NO:107. For example, a protein-modulating polypeptide can have an amino acid sequence with at least 60% sequence identity, e.g., 60%, 65%, 70%, 75%, 80%, 85%, 90%, 95%, 97%, 98%, or 99% sequence identity, to the amino acid sequence set forth in SEQ ID NO:107.

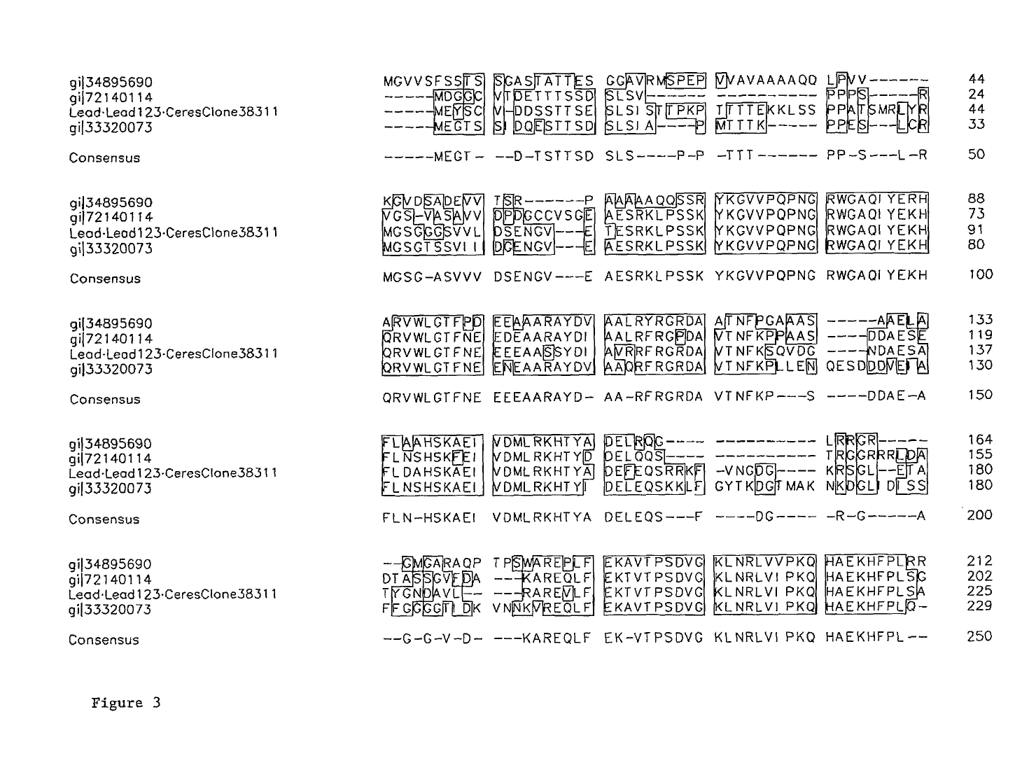

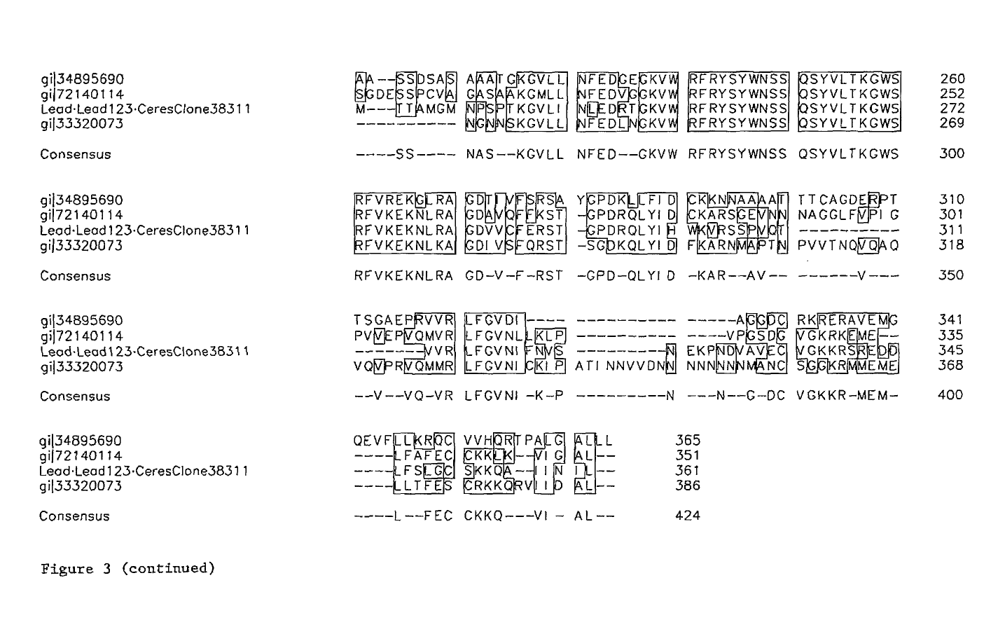

Amino acid sequences of homologs and/or orthologs of the polypeptide having the amino acid sequence set forth in SEQ ID NO:107 are provided in FIG. 3, along with a consensus sequence. A consensus amino acid sequence for such homologs and/or orthologs was determined by aligning amino acid sequences, e.g., amino acid sequences related to SEQ ID NO:107, from a variety of species and determining the most common amino acid or type of amino acid at each position. For example, the alignment in FIG. 3 provides the amino acid sequences of Ceres Clone 38311 (SEQ ID NO:107), gi|72140114 (SEQ ID NO:109), gi|33320073 (SEQ ID NO:110), and gi|34895690 (SEQ ID NO:112). Other homologs and/or orthologs include Ceres CLONE ID no. 19561 (SEQ ID NO:108), Ceres CLONE ID no. 597624 (SEQ ID NO:111), and Ceres Clone:1464039 (SEQ ID NO:236).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:108, SEQ ID NO:109, SEQ ID NO:110, SEQ ID NO:111, SEQ ID NO:112, or the consensus sequence set forth in FIG. 3.

A protein-modulating polypeptide can have a DUF569 domain characteristic of a polypeptide of unknown function. SEQ ID NO:114 sets forth the amino acid sequence of an Arabidopsis clone, identified herein as Ceres Clone 109289 (SEQ ID NO:113), that is predicted to encode a polypeptide of unknown function. A protein-modulating polypeptide can comprise the amino acid sequence set forth in SEQ ID NO:114. Alternatively, a protein-modulating polypeptide can be a homolog, ortholog, or variant of the polypeptide having the amino acid sequence set forth in SEQ ID NO:114. For example, a protein-modulating polypeptide can have an amino acid sequence with at least 30% sequence identity, e.g., 30%, 35%, 40%,45%, 50%, 55%, 60%, 65%, 70%, 75%, 80%, 85%, 90%, 95%, 97%, 98%, or 99% sequence identity, to the amino acid sequence set forth in SEQ ID NO:114.

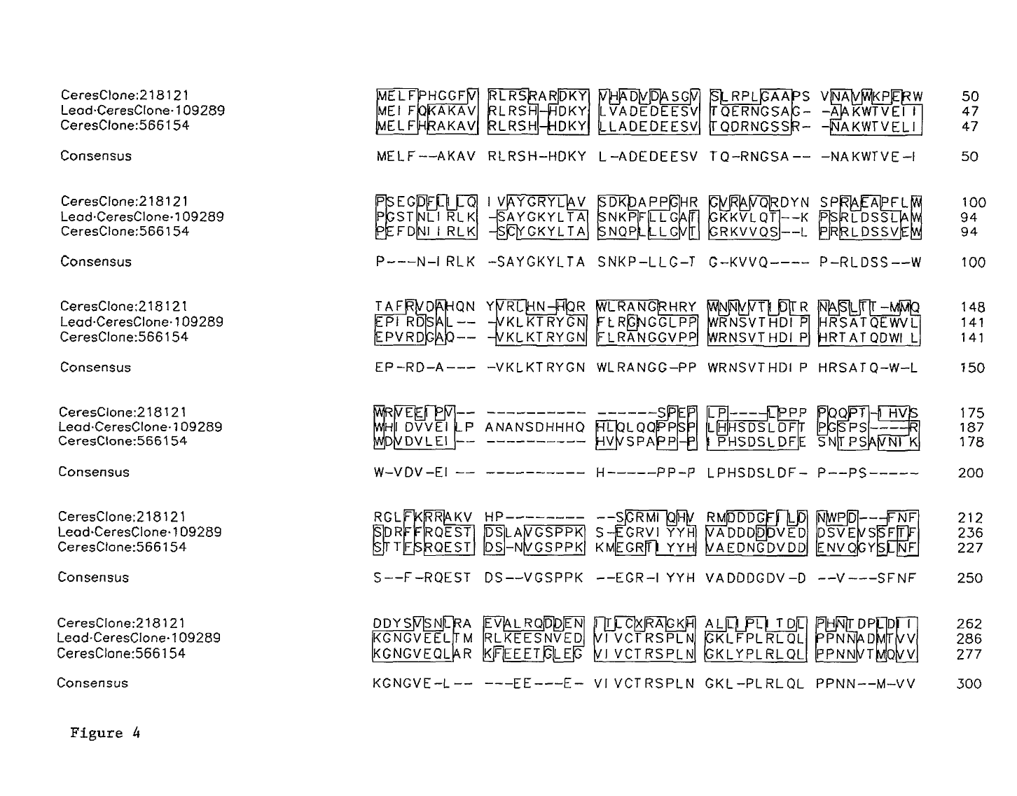

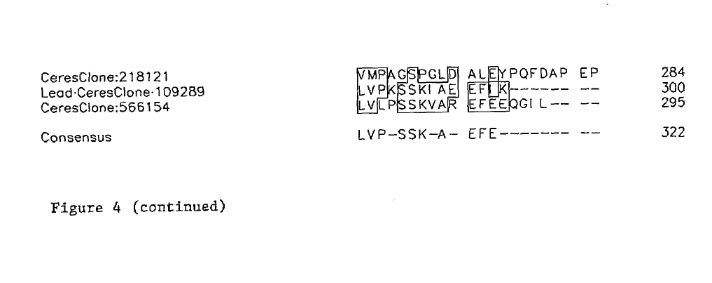

Amino acid sequences of homologs and/or orthologs of the polypeptide having the amino acid sequence set forth in SEQ ID NO:114 are provided in FIG. 4, along with a consensus sequence. A consensus amino acid sequence for such homologs and/or orthologs was determined by aligning amino acid sequences, e.g., amino acid sequences related to SEQ ID NO:114, from a variety of species and determining the most common amino acid or type of amino acid at each position. For example, the alignment in FIG. 4 provides the amino acid sequences of Ceres Clone 109289 (SEQ ID NO:114), Ceres Clone:566154 (SEQ ID NO:115) and Ceres Clone:218121 (SEQ ID NO:117). Other homologs and/or orthologs include Ceres CLONE ID no. 541790 (SEQ ID NO:116) and Ceres Clone:1459859 (SEQ ID NO:252).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:115, SEQ ID NO:116, SEQ ID NO:117, SEQ ID NO:252, or the consensus sequence set forth in FIG. 4.

A protein-modulating polypeptide can be a nuclear polypeptide, such as a XAP5 polypeptide. XAP5 polypeptides are found in a wide range of eukaryotes and may have DNA binding activity. SEQ ID NO:119 sets forth the amino acid sequence of an Arabidopsis clone, identified herein as Ceres Clone 19342 (SEQ ID NO:118), that is predicted to encode a XAP5 polypeptide. A protein-modulating polypeptide can comprise the amino acid sequence set forth in SEQ ID NO:119. Alternatively, a protein-modulating polypeptide can be a homolog, ortholog, or variant of the polypeptide having the amino acid sequence set forth in SEQ ID NO:119. For example, a protein-modulating polypeptide can have an amino acid sequence with at least 70% sequence identity, e.g., 70%, 75%, 80%, 85%, 90%, 95%, 97%, 98%, or 99% sequence identity, to the amino acid sequence set forth in SEQ ID NO:119.

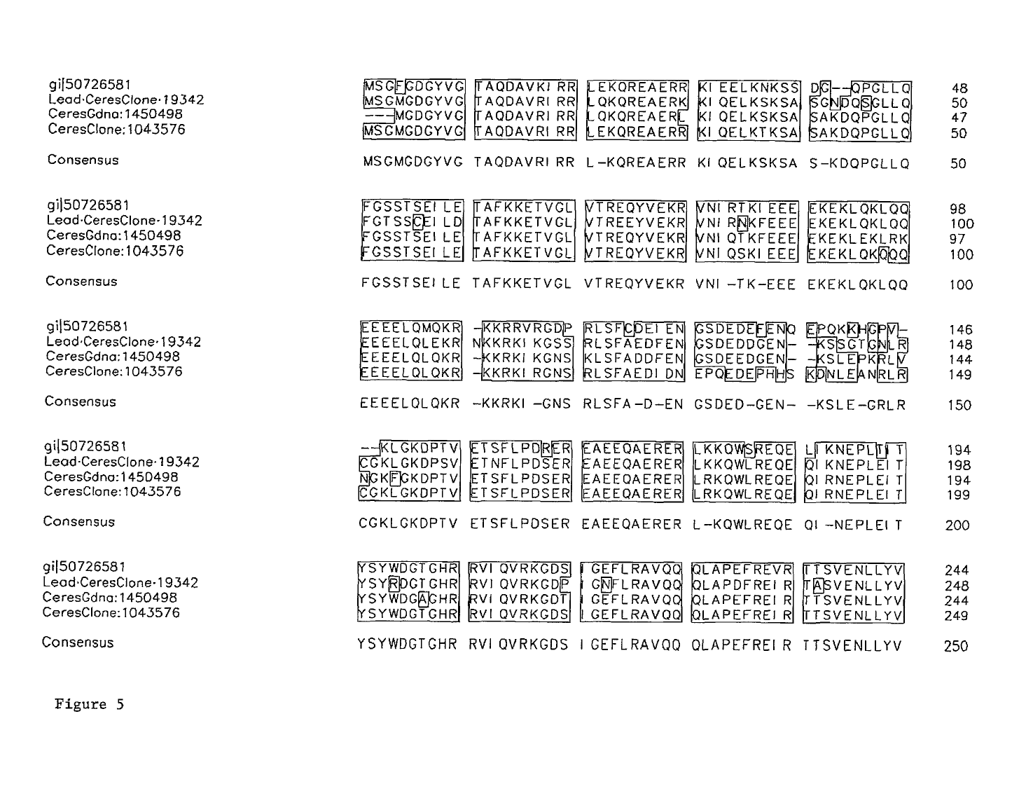

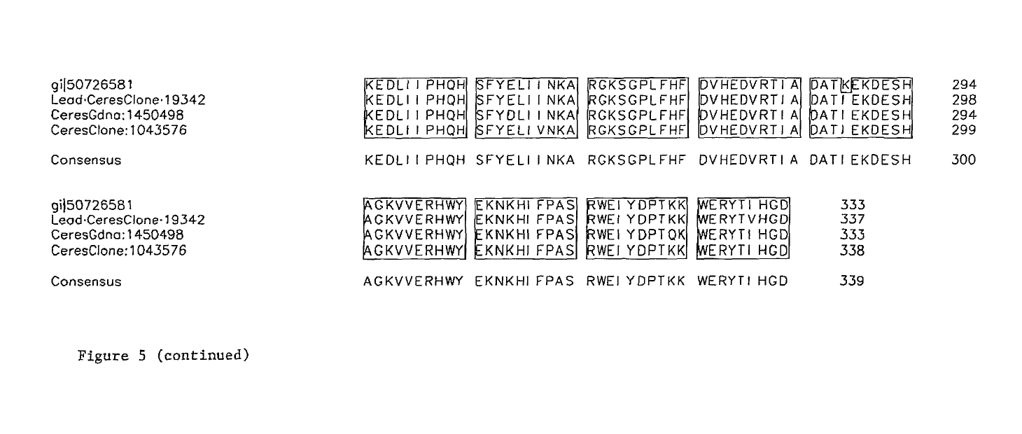

Amino acid sequences of homologs and/or orthologs of the polypeptide having the amino acid sequence set forth in SEQ ID NO:119 are provided in FIG. 5, along with a consensus sequence. A consensus amino acid sequence for such homologs and/or orthologs was determined by aligning amino acid sequences, e.g., amino acid sequences related to SEQ ID NO:119, from a variety of species and determining the most common amino acid or type of amino acid at each position. For example, the alignment in FIG. 5 provides the amino acid sequences of Ceres Clone 19342 (SEQ ID NO:119), Ceres Annot:1450498 (SEQ ID NO:121), Ceres Clone:1043576 (SEQ ID NO:124), and gi|50726581 (SEQ ID NO:125). Other homologs and/or orthologs include Ceres Annot:1460687 (SEQ ID NO:123).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:121, SEQ ID NO:123, SEQ ID NO:124, SEQ ID NO:125, or the consensus sequence set forth in FIG. 5.

A protein-modulating polypeptide can be an electron carrier polypeptide, such as glutaredoxin polypeptide. Glutaredoxin polypeptides, also known as thioltransferase polypeptides, are small polypeptides of approximately one hundred amino-acid residues. Glutaredoxin polypeptides function as electron carriers in the glutathione-dependent synthesis of deoxyribonucleotides by the enzyme ribonucleotide reductase. Like thioredoxin polypeptides, which function in a similar way, glutaredoxin polypeptides possess an active center disulphide bond. A glutaredoxin polypeptide exists in either a reduced or an oxidized form where two cysteine residues are linked in an intramolecular disulphide bond. SEQ ID NO:127 sets forth the amino acid sequence of an Arabidopsis clone, identified herein as Ceres Clone 21006 (SEQ ID NO:126), that is predicted to encode a glutaredoxin polypeptide. A protein-modulating polypeptide can comprise the amino acid sequence set forth in SEQ ID NO:127. Alternatively, a protein-modulating polypeptide can be a homolog, ortholog, or variant of the polypeptide having the amino acid sequence set forth in SEQ ID NO:127. For example, a protein-modulating polypeptide can have an amino acid sequence with at least 50% sequence identity, e.g., 50%, 55%, 60%, 65%, 70%, 75%, 80%, 85%, 90%, 95%, 97%, 98%, or 99% sequence identity, to the amino acid sequence set forth in SEQ ID NO:127.

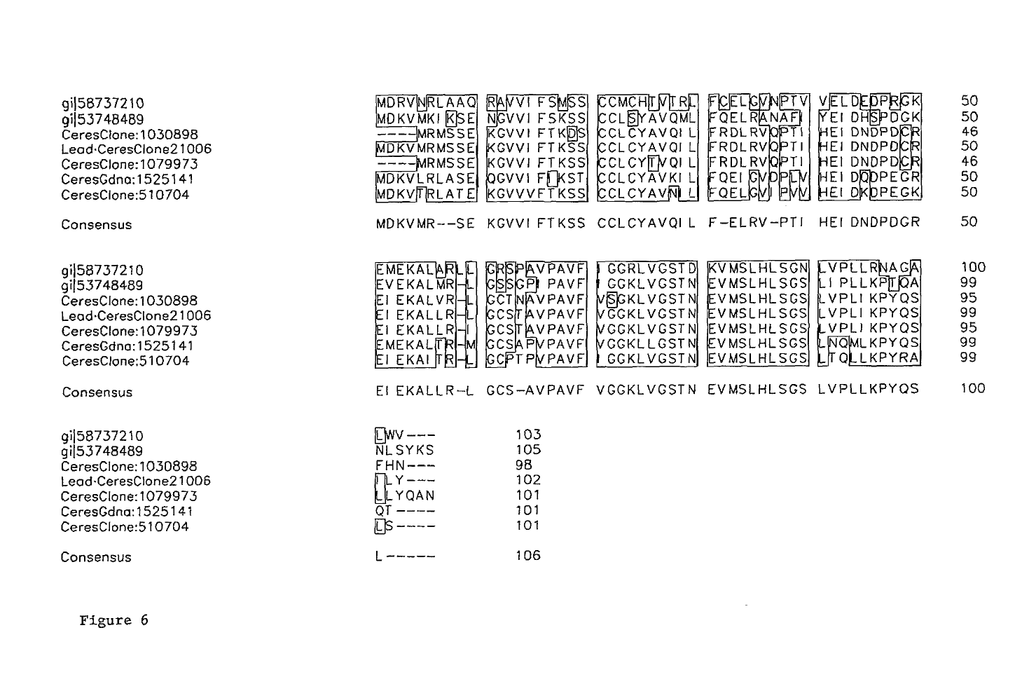

Amino acid sequences of homologs and/or orthologs of the polypeptide having the amino acid sequence set forth in SEQ ID NO:127 are provided in FIG. 6, along with a consensus sequence. A consensus amino acid sequence for such homologs and/or orthologs was determined by aligning amino acid sequences, e.g., amino acid sequences related to SEQ ID NO:127, from a variety of species and determining the most common amino acid or type of amino acid at each position. For example, the alignment in FIG. 6 provides the amino acid sequences of Ceres Clone 21006 (SEQ ID NO:127), Ceres Clone:1079973 (SEQ ID NO:128), Ceres Clone:1030898 (SEQ ID NO:131), Ceres Clone:510704 (SEQ ID NO:139), Ceres Annot:1525141 (SEQ ID NO:141), gi|53748489 (SEQ ID NO:144), and giÅ58737210 (SEQ ID NO:145). Other homologs and/or orthologs include Public GI no. 7573425 (SEQ ID NO:129), Ceres CLONE ID no. 953083 (SEQ ID NO:130), Ceres CLONE ID no. 940212 (SEQ ID NO:132), Ceres CLONE ID no. 1070065 (SEQ ID NO:133), Ceres CLONE ID no. 125679 (SEQ ID NO:134), Public GI no. 21537263 (SEQ ID NO:135), Public GI no. 24111317 (SEQ ID NO:136), Ceres CLONE ID no. 39560 (SEQ ID NO:137), Ceres CLONE ID no. 871147 (SEQ ID NO:138), Ceres Annot:1472813 (SEQ ID NO:143), Public GI no. 77556540 (SEQ ID NO:146), Ceres Clone: 1448879 (SEQ ID NO:240), Ceres Clone:1490481 (SEQ ID NO:242), Ceres Clone:1856294 (SEQ ID NO:244), Ceres Clone:100028679 (SEQ ID NO:246), Ceres Clone:1629347 (SEQ ID NO:248), and Ceres Clone:1768062 (SEQ ID NO:250).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:141, SEQ ID NO:143, SEQ ID NO:144, SEQ ID NO:145, SEQ ID NO:146, SEQ ID NO:240, SEQ ID NO:242, SEQ ID NO:244, SEQ ID NO:246, SEQ ID NO:248, SEQ ID NO:250, or the consensus sequence set forth in FIG. 6.

A protein-modulating polypeptide can have a PQ loop repeat. This repeated motif of unknown function has been found between the transmembrane helices of cystinosin, yeast ERS1, and mannose-P-dolichol utilization defect 1. The positioning of this repeat suggests that it may be associated with glycosylation machinery. SEQ ID NO:148 sets forth the amino acid sequence of an Arabidopsis clone, identified herein as Ceres Clone 2296 (SEQ ID NO:147), that is predicted to encode a polypeptide having a PQ loop repeat. A protein-modulating polypeptide can comprise the amino acid sequence set forth in SEQ ID NO:148. Alternatively, a protein-modulating polypeptide can be a homolog, ortholog, or variant of the polypeptide having the amino acid sequence set forth in SEQ ID NO:148. For example, a protein-modulating polypeptide can have an amino acid sequence with at least 60% sequence identity, e.g., 60%, 65%, 70%, 75%, 80%, 85%, 90%, 95%, 97%, 98%, or 99% sequence identity, to the amino acid sequence set forth in SEQ ID NO:148.

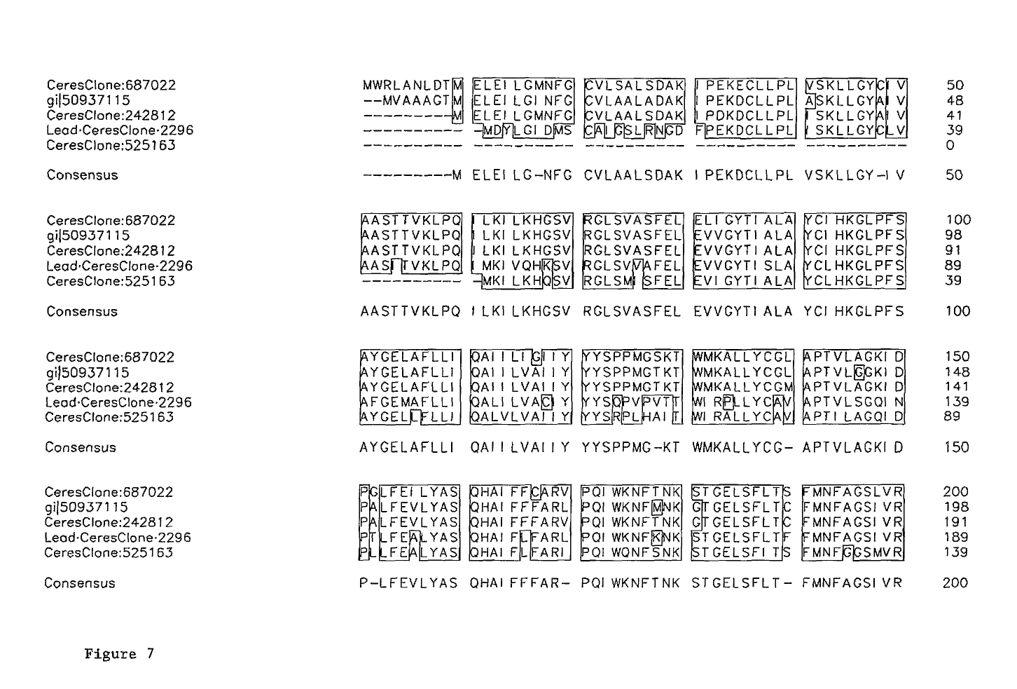

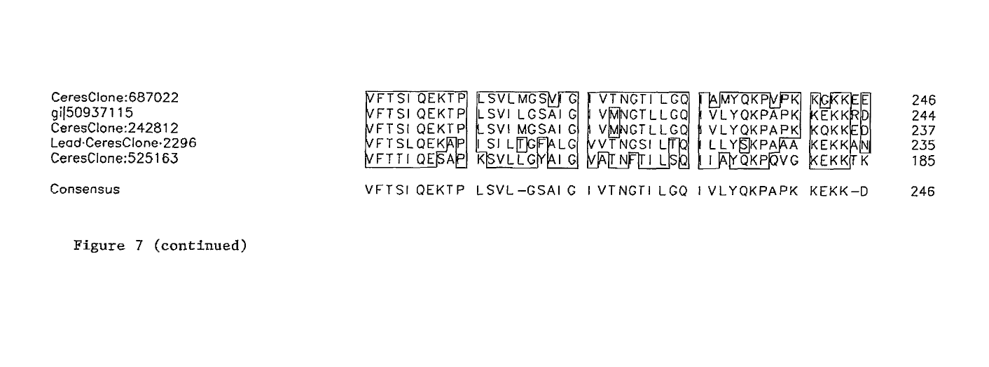

Amino acid sequences of homologs and/or orthologs of the polypeptide having the amino acid sequence set forth in SEQ ID NO:148 are provided in FIG. 7, along with a consensus sequence. A consensus amino acid sequence for such homologs and/or orthologs was determined by aligning amino acid sequences, e.g., amino acid sequences related to SEQ ID NO:148, from a variety of species and determining the most common amino acid or type of amino acid at each position. For example, the alignment in FIG. 7 provides the amino acid sequences of Ceres Clone 2296 (SEQ ID NO:148), Ceres Clone:525163 (SEQ ID NO:149), gi|50937115 (SEQ ID NO:150), Ceres Clone:242812 (SEQ ID NO:151), and Ceres Clone:687022 (SEQ ID NO:153). Other homologs and/or orthologs include Ceres CLONE ID no. 243125 (SEQ ID NO:152) and Ceres Clone:1937560 (SEQ ID NO:238).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:149, SEQ ID NO:150, SEQ ID NO:151, SEQ ID NO:152, SEQ ID NO:153, SEQ ID NO:238, or the consensus sequence set forth in FIG. 7.

A protein-modulating polypeptide can have a heavy metal associated (HMA) domain characteristic of polypeptides that transport heavy metals. An HMA domain contains two conserved cysteine residues that may be involved in metal binding. SEQ ID NO:155 sets forth the amino acid sequence of an Arabidopsis clone, identified herein as Ceres Clone 33038 (SEQ ID NO:154), that is predicted to encode a polypeptide having an HMA domain. A protein-modulating polypeptide can comprise the amino acid sequence set forth in SEQ ID NO:155. Alternatively, a protein-modulating polypeptide can be a homolog, ortholog, or variant of the polypeptide having the amino acid sequence set forth in SEQ ID NO:155. For example, a protein-modulating polypeptide can have an amino acid sequence with at least 70% sequence identity, e.g., 70%, 75%, 80%, 85%, 90%, 95%, 97%, 98%, or 99% sequence identity, to the amino acid sequence set forth in SEQ ID NO:155.

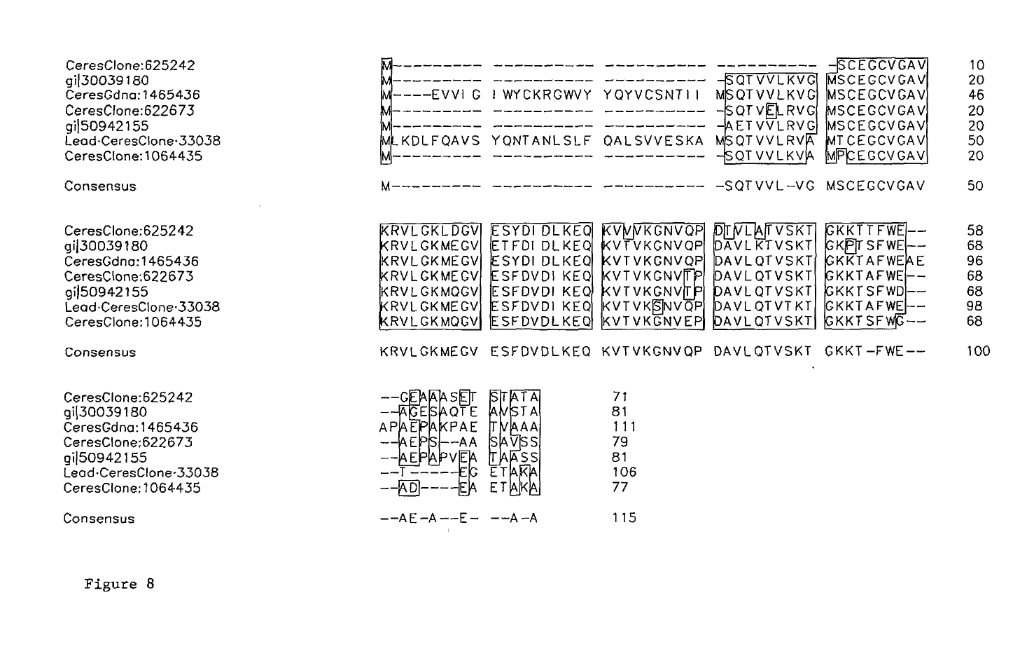

Amino acid sequences of homologs and/or orthologs of the polypeptide having the amino acid sequence set forth in SEQ ID NO:155 are provided in FIG. 8, along with a consensus sequence. A consensus amino acid sequence for such homologs and/or orthologs was determined by aligning amino acid sequences, e.g., amino acid sequences related to SEQ ID NO:155, from a variety of species and determining the most common amino acid or type of amino acid at each position. For example, the alignment in FIG. 8 provides the amino acid sequences of Ceres Clone 33038 (SEQ ID NO:155), Ceres Clone:1064435 (SEQ ID NO:157), Ceres Clone:622673 (SEQ ID NO:158), Ceres Annot:1465436 (SEQ ID NO:160), gi|30039180 (SEQ ID NO:162), Ceres Clone:625242 (SEQ ID NO:163), and gi|50942155 (SEQ ID NO:165). Other homologs and/or orthologs include Public GI no. 18655401 (SEQ ID NO:156), Public GI no. 47176684 (SEQ ID NO:161), Ceres CLONE ID no. 944316 (SEQ ID NO:164), Ceres Clone:100063116 (SEQ ID NO:254), Ceres Clone:1771295 (SEQ ID NO:256), and Ceres Clone:1609456 (SEQ ID NO:258).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:156, SEQ ID NO:157, SEQ ID NO:158, SEQ ID NO:160, SEQ ID NO:161, SEQ ID NO:162, SEQ ID NO:163, SEQ ID NO:164, SEQ ID NO:165, SEQ ID NO:254, SEQ ID NO:256, SEQ ID NO:258, or the consensus sequence set forth in FIG. 8.

A protein-modulating polypeptide can have a UQ_CON domain characteristic of an ubiquitin-conjugating enzyme. An ubiquitin-conjugating enzyme (E2) is one of at least three enzymes involved in ubiquitinylation. The E2 enzyme transfers a ubiquitin moiety directly to a substrate, or to a ubiquitin ligase (E3). E2 enzymes are broadly grouped into four classes: class I enzymes possess the catalytic core domain (UBC) containing the active site cysteine, class II enzymes possess a UBC and a C-terminal extension, class III enzymes possess a UBC and an N-terminal extension, and class IV enzymes possess a UTBC and both N- and C-terminal extensions. These extensions appear to be important for some subfamily function, including E2 localization and protein-protein interactions. In addition, there are proteins with an E2-like fold that are devoid of catalytic activity, but which appear to assist in poly-ubiquitin chain formation. SEQ ID NO:167 sets forth the amino acid sequence of an Arabidopsis clone, identified herein as Ceres Clone 5821 (SEQ ID NO:166), that is predicted to encode a ubiquitin-conjugating enzyme. A protein-modulating polypeptide can comprise the amino acid sequence set forth in SEQ ID NO:167. Alternatively, a protein-modulating polypeptide can be a homolog, ortholog, or variant of the polypeptide having the amino acid sequence set forth in SEQ ID NO:167. For example, a protein-modulating polypeptide can have an amino acid sequence with at least 65% sequence identity, e.g., 65%, 70%, 75%, 80%, 85%, 90%, 95%, 97%, 98%, or 99% sequence identity, to the amino acid sequence sot forth in SEQ ID NO:167.

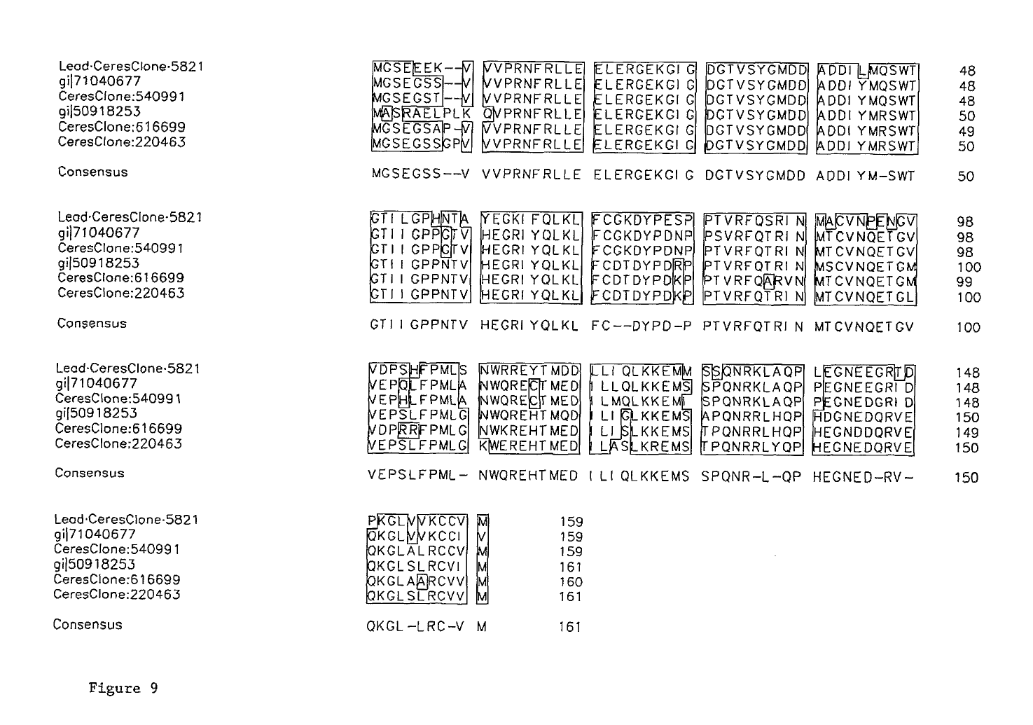

Amino acid sequences of homologs and/or orthologs of the polypeptide having the amino acid sequence set forth in SEQ ID NO:167 are provided in FIG. 9, along with a consensus sequence. A consensus amino acid sequence for such homologs and/or orthologs was determined by aligning amino acid sequences, e.g., amino acid sequences related to SEQ ID NO:167, from a variety of species and determining the most common amino acid or type of amino acid at each position. For example, the alignment in FIG. 9 provides the amino acid sequences of Ceres Clone 5821 (SEQ ID NO:167), gi|71040677 (SEQ ID NO:170), Ceres Clone:540991 (SEQ ID NO:171), gi|50918253 (SEQ ID NO:172), Ceres Clone:616699 (SEQ ID NO:173), and Ceres Clone:220463 (SEQ ID NO:175). Other homologs and/or orthologs include Public GI no. 28827264 (SEQ ID NO:168), Public GI no. 20259984 (SEQ ID NO:169), Ceres CLONE ID no. 677401 (SEQ ID NO:174), Ceres Clone:980825 (SEQ ID NO:220), Ceres Clone:1850191 (SEQ ID NO:222), Ceres Clone:1838128 (SEQ ID NO:224), Ceres Clone:1512371 (SEQ ID NO:226), and Ceres Clone:1767492 (SEQ ID NO:228).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:220, SEQ ID NO:222, SEQ ID NO:224, SEQ ID NO:226, SEQ ID NO:228, or the consensus sequence set forth in FIG. 9.

A protein-modulating polypeptide encoded by a recombinant nucleic acid can be a native protein-modulating polypeptide, i.e., one or more additional copies of the coding sequence for a protein-modulating polypeptide that is naturally present in the cell. Alternatively, a protein-modulating polypeptide can be heterologous to the cell, e.g., a transgenic Lycopersicon plant can contain the coding sequence for a transcription factor polypeptide from a Glycine plant.

A protein-modulating polypeptide can include additional amino acids that are not involved in protein modulation, and thus can be longer than would otherwise bc the case. For example, a protein-modulating polypeptide can include an amino acid sequence that functions as a reporter. Such a protein-modulating polypeptide can be a fusion protein in which a green fluorescent protein (GFP) polypeptide is fused to, e.g., SEQ ID NO:81, or in which a yellow fluorescent protein (YFP) polypeptide is fused to, e.g., SEQ ID NO:83. In some embodiments, a protein-modulating polypeptide includes a purification tag, a chloroplast transit peptide, a mitochondrial transit peptide, or a leader sequence added to the amino or carboxy terminus.

Protein-modulating polypeptide candidates suitable for use in the invention can be identified by analysis of nucleotide and polypeptide sequence alignments. For example, performing a query on a database of nucleotide or polypeptide sequences can identify homologs and/or orthologs of protein-modulating polypeptides. Sequence analysis can involve BLAST, Reciprocal BLAST, or PSI-BLAST analysis of nonredundant databases using known protein-modulating polypeptide amino acid sequences. Those polypeptides in the database that have greater than 30% sequence identity can be identified as candidates for further evaluation for suitability as a protein-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in protein-modulating polypeptides, e.g., conserved functional domains.

The identification of conserved regions in a template or subject polypeptide can facilitate production of variants of wild type protein-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999).

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within protein-modulating polypeptides. These conserved regions can be useful in identifying functionally similar (orthologous) protoin-modulating polypeptides.

In some instances, suitable protein-modulating polypeptides can be synthesized on the basis of consensus functional domains and/or conserved regions in polypeptides that are homologous protein-modulating polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of protein-modulating polypeptides are shown in FIGS. 1-9. Each Figure represents an alignment of the amino acid sequence of a protein-modulating polypeptide with the amino acid sequences of corresponding homologs and/or orthologs. Amino acid sequences of protein-modulating polypeptides and their corresponding homologs and/or orthologs have been aligned to identify conserved amino acids and to determine consensus sequences that contain frequently occurring amino acid residues at particular positions in the aligned sequences, as shown in FIGS. 1-9. A dash in an aligned sequence represents a gap, i.e., a lack of an amino acid at that position. Identical amino acids or conserved amino acid substitutions among aligned sequences are identified by boxes.

Each consensus sequence is comprised of conserved regions. Each conserved region contains a sequence of contiguous amino acid residues. A dash in a consensus sequence indicates that the consensus sequence either lacks an amino acid at that position or includes an amino acid at that position. If an amino acid is present, the residue at that position corresponds to one found in any aligned sequence at that position.

Useful polypeptides can be constructed based on the consensus sequence in FIG. 1, FIG. 2, FIG. 3, FIG. 4, FIG. 5, FIG. 6, FIG. 7, FIG. 8, or FIG. 9. Such a polypeptide includes the conserved regions in the selected consensus sequence, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Consensus domains and conserved regions can be identified by homologous polypeptide sequence analysis as described above. The suitability of polypeptides for use as protein-modulating polypeptides can be evaluated by functional complementation studies.

### Nucleic Acids

Isolated nucleic acids are provided herein. The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

Nucleic acids described herein include protein-modulating nucleic acids. Protein-modulating nucleic acids can be effective to modulate protein levels when transcribed in a plant or plant cell. A protein-modulating nucleic acid can comprise the nucleotide sequence set forth in SEQ ID NO:80, SEQ ID NO:82, SEQ ID NO:87, SEQ ID NO:94, SEQ ID NO:98, SEQ ID NO:106, SEQ ID NO:113, SEQ ID NO:118, SEQ ID NO:120, SEQ ID NO:122, SEQ ID NO:126, SEQ ID NO:140, SEQ ID NO:142, SEQ ID NO:147, SEQ ID NO:154, SEQ ID NO:159, SEQ ID NO:166, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:217, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:249, SEQ ID NO:251, SEQ ID NO:253, SEQ ID NO:255, SEQ ID NO:257, SEQ ID NO:274, SEQ ID NO:275, SEQ ID NO:276, SEQ ID NO:277, SEQ ID NO:278, or SEQ ID NO:279. Alternatively, a protein-modulating nucleic acid can be a variant of the nucleic acid having the nucleotide sequence set forth in SEQ ID NO: SEQ ID NO:80, SEQ ID NO:82, SEQ ID NO:87, SEQ ID NO:94, SEQ ID NO:98, SEQ ID NO:106, SEQ ID NO:113, SEQ ID NO:118, SEQ ID NO:120, SEQ ID NO:122, SEQ ID NO:126, SEQ ID NO:140, SEQ ID NO:142, SEQ ID NO:147, SEQ ID NO:154, SEQ ID NO:159, SEQ ID NO:166, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:217, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:249, SEQ ID NO:251, SEQ ID NO:253, SEQ ID NO:255, SEQ ID NO:257, SEQ ID NO:274, SEQ ID NO:275, SEQ ID NO:276, SEQ ID NO:277, SEQ ID NO:278, or SEQ ID NO:279. For example, a protein-modulating nucleic acid can have a nucleotide sequence with at least 80% sequence identity, e.g., 81%, 85%, 90%, 95%, 97%, 98%, or 99% sequence identity, to the nucleotide sequence sect forth in SEQ ID NO: SEQ ID NO:80, SEQ ID NO:82, SEQ ID NO:87, SEQ ID NO:94, SEQ ID NO:98, SEQ ID NO:106, SEQ ID NO:113, SEQ ID NO:118, SEQ ID NO:120, SEQ ID NO:122, SEQ ID NO:126, SEQ ID NO:140, SEQ ID NO:142, SEQ ID NO:147, SEQ ID NO:154, SEQ ID NO:159, SEQ ID NO:166, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:217, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:249, SEQ ID NO:251, SEQ ID NO:253, SEQ ID NO:255, SEQ ID NO:257, SEQ ID NO:274, SEQ ID NO:275, SEQ ID NO:276, SEQ ID NO:277, SEQ ID NO:278, or SEQ ID NO:279.

An “isolated nucleic acid” can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can bc used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated. into a vector. Isolated, nucleic acids of the invention also can be obtained by mutagenesis of, e.g., a naturally occurring DNA.

As used herein, the term “percent sequence identity” refers to the degree of identity between any given query sequence, e.g., SEQ ID NO:81, and a subject sequence. A subject sequence typically has a length that is from 80 percent to 200 percent of the length of the query sequence, e.g., 82, 85, 87, 89, 90, 93, 95, 97, 99, 100, 105, 110, 115, 120, 130, 140, 150, 160, 170, 180, 190, or 200 percent of the length of the query sequence. A percent identity for any subject nucleic acid or polypeptide relative to a query nucleic acid or polypeptide can be determined as follows. A query sequence (e.g., a nucleic acid sequence or an amino acid sequence) is aligned to one or more subject sequences using the computer program ClustalW (version 1.83, default parameters), which allows alignments of nucleic acid or polypeptide sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a subject nucleic acid or amino acid sequence to a query sequence, the sequences are aligned using ClustalW, the number of identical matches in the alignment is divided by the length of the query sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to modulate protein levels. A recombinant nucleic acid construct can comprise a nucleic acid encoding a protein-modulating polypeptide as described herein, operably linked to a regulatory region suitable for expressing the protein-modulating polypeptide in the plant or cell. Thus, a nucleic acid can comprise a coding sequence that encodes any of the protein-modulating polypeptides as set forth in SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, and the consensus sequences set forth in FIGS. 1-9. Examples of nucleic acids encoding protein-modulating polypeptides are set forth in SEQ ID NO: SEQ ID NO:80, SEQ ID NO:82, SEQ ID NO:87, SEQ ID NO:94, SEQ ID NO:98, SEQ ID NO:106, SEQ ID NO:113, SEQ ID NO:118, SEQ ID NO:120, SEQ ID NO:122, SEQ ID NO:126, SEQ ID NO:140, SEQ ID NO:142, SEQ ID NO:147, SEQ ID NO:154, SEQ ID NO:159, SEQ ID NO:166, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:217, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:249, SEQ ID NO:251, SEQ ID NO:253, SEQ ID NO:255, SEQ ID NO:257, SEQ ID NO:274, SEQ ID NO:275, SEQ ID NO:276, SEQ ID NO:277, SEQ ID NO:278, and SEQ ID NO:279.

In some cases, a recombinant nucleic acid construct can include a nucleic acid comprising less than the full-length of a coding sequence. Typically, such a construct also includes a regulatory region operably linked to the protein-modulating nucleic acid. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given protein-modulating polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence.

Some suitable regulatory regions initiate transcription only, or predominantly, in certain cell types, for example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat). Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of regulatory regions are described below. Some of the regulatory regions indicated below as well as additional regulatory regions are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/034308; and PCT/US05/23639. Nucleotide sequences of promoters are set forth in SEQ ID NOs:1-79 and 259-274. It will be appreciated that a regulatory region may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:76), YP0144 (SEQ ID NO:55), YP0190 (SEQ ID NO:59), p13879 (SEQ ID NO:75), YP0050 (SEQ ID NO:35), p32449 (SEQ ID NO:77),21876 (SEQ ID NO:1), YP0158 (SEQ ID NO:57), YP0214 (SEQ ID NO:61), YP0380 (SEQ ID NO:70), PT0848 (SEQ ID NO:26), and PT0633 (SEQ ID NO:7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO:52), YP0275 (SEQ ID NO:63), PT0625 (SEQ ID NO:6), PT0660 (SEQ ID NO:9), PT0683 (SEQ ID NO:14), and PT0758 (SEQ ID NO:22) promoters. Other root-preferential promoters include the PT0613 (SEQ ID NO:5), PT0672 (SEQ ID NO:11), PT0688 (SEQ ID NO:15), and PT0837 (SEQ ID NO:24) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in -maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombc et al., Plant Physiol., 104(4):167-176 (1994)), the soybean a subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092 (SEQ ID NO:38), PT0676 (SEQ ID NO:12), and PT0708 (SEQ ID NO:17) promoters.

Ovary Tissue Promoters

Promoters that are active in ovary tissues such as the ovule wall and mesocarp can also be useful, e.g., a polygalacturonidase promoter, the banana TRX promoter, the melon actin promoter, YP0396 (SEQ ID NO:74), and PT0623 (SEQ ID NO:273). Examples of promoters that are active primarily in ovules include YP0007 (SEQ ID NO:30), YP0111 (SEQ ID NO:46), YP0092 (SEQ ID NO:38), YP0103 (SEQ ID NO:43), YP0028 (SEQ ID NO:33), YP0121 (SEQ ID NO:51), YP0008 (SEQ ID NO:31), YP0039 (SEQ ID NO:34), YP0115 (SEQ ID NO:47), YP0119 (SEQ ID NO:49), YP0120 (SEQ ID NO:50), and YP0374 (SEQ ID NO:68).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO:34), YP0101 (SEQ ID NO:41), YP0102 (SEQ ID NO:42), YP0110 (SEQ ID NO:45), YP0117 (SEQ ID NO:48), YP0119 (SEQ ID NO:49), YP0137 (SEQ ID NO:53), DME, YP0285 (SEQ ID NO:64), and YP0212 (SEQ ID NO:60). Other promoters that may be useful include the following rice promoters: p530c10 (SEQ ID NO:259), pOsFIE2-2 (SEQ ID NO:260), pOsMEA (SEQ ID NO:261), pOsYp102 (SEQ ID NO:262), and pOsYp285 (SEQ ID NO:263).

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep (2001) 20:647-654), YP0097 (SEQ ID NO:40), YP0107 (SEQ ID NO:44), YP0088 (SEQ ID NO:37), YP0143 (SEQ ID NO:54), YP0156 (SEQ ID NO:56), PT0650 (SEQ ID NO:8), PT0695 (SEQ ID NO:16), PT0723 (SEQ ID NO:19), PT0838 (SEQ ID NO:25), PT0879 (SEQ ID NO:28), and PT0740 (SEQ ID NO:20).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), PR0924 (SEQ ID NO:78), YP0144 (SEQ ID NO:55), YP0380 (SEQ ID NO:70), and PT0585 (SEQ ID NO:4).

Vascular Tissue Promoters

Examples of promoters that have high or preferential activity in vascular bundles include YP0087 (SEQ ID NO:266), YP0093 (SEQ ID NO:267), YP0108 (SEQ ID NO:268), YP0022 (SEQ ID NO:269), and YP0080 (SEQ ID NO:270). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO:70), PT0848 (SEQ ID NO:26), YP0381 (SEQ ID NO:71), YP0337 (SEQ ID NO:66), PT0633 (SEQ ID NO:7), YP0374 (SEQ ID NO:68), PT0710 (SEQ ID NO:18), YP0356 (SEQ ID NO:67), YP0385 (SEQ ID NO:73), YP0396 (SEQ ID NO:74), YP0388 (SEQ ID NO:271), YP0384 (SEQ ID NO:72), PT0688 (SEQ ID NO:15), YP0286 (SEQ ID NO:65), YP0377 (SEQ ID NO:69), PD1367 (SEQ ID NO:79), and PD0901 (SEQ ID NO:272). Nitrogen-inducible promoters include PT0863 (SEQ ID NO:27), PT0829 (SEQ ID NO:23), PT0665 (SEQ ID NO:10), and PT0886 (SEQ ID NO:29). Example of a shade-inducible promoters are PR0924 (SEQ ID NO:78) and PT0678 (SEQ ID NO:13).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, shoot-preferential, callus-preferential, trichome cell-preferential, guard cell-preferential such as PT0678 (SEQ ID NO:13), tuber-preferential, parenchyma cell-preferential, and senescence-preferential promoters. Promoters designated YP0086 (SEQ ID NO:36), YP0188 (SEQ ID NO:58), YP0263 (SEQ ID NO:62), PT0758 (SEQ ID NO:22), PT0743 (SEQ ID NO:21), PT0829 (SEQ ID NO:23), YP0119 (SEQ ID NO:49), and YP0096 (SEQ ID NO:39), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a protein-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can bc propagated vegetatively for those species amenable to such techniques. As used herein, a transgenic plant also refers to progeny of an initial transgenic plant. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F2, F3, F4, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous protein-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

In aspects related to making transgenic plants, a typical step involves selection or screening of transformed plants, e.g., for the presence of a functional vector as evidenced by expression of a selectable marker. Selection or screening can be carried out among a population of recipient cells to identify transformants using selectable marker genes such as herbicide resistance genes. Physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

A population of transgenic plants can be screened and/or selected for those members of the population that have a desired trait or phenotype conferred by expression of the transgene. For example, a population of progeny of a single transformation event can be screened for those plants having a desired level of expression of a heterologous protein-modulating polypeptide or nucleic acid. As an alternative, a population of plants comprising independent transformation events can be screened for those plants having a desired trait, such as a modulated level of protein. Selection and/or screening can be carried out over one or more generations, which can be useful to identify those plants that have a statistically significant difference in a protein level as compared to a corresponding level in a control plant. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is expected to be exhibited by the plant. Selection and/or screening can be carried out to choose those transgenic plants having a statistically significant difference in a protein level relative to a control plant that lacks the transgene. Selected or screened transgenic plants have an altered phenotype as compared to a corresponding control plant, as described in the “Transgenic Plant Phenotypes” section below.

### Plant Species

The polynucleotides and vectors described herein can be used to transform a number of monocotyledonous and dicotyledonous plants and plant cell systems, including dicots such as alfalfa, almond, amaranth, apple, beans (including kidney beans, lima beans, dry beans, green beans), brazil nut, broccoli, cabbage, carrot, cashew, castor bean, cherry, chick peas, chicory, clover, cocoa, coffee, cotton, crambe, flax, grape, grapefruit, hazelnut, lemon, lentils, lettuce, linseed, macadamia nut, mango, melon (e.g., watermelon, cantaloupe), mustard, orange, peach, peanut, pear, peas, pecan, pepper, pistachio, plum, potato, oilseed rape, quinoa, rapeseed (high erucic acid and canola), safflower, sesame, soybean, spinach, strawberry, sugar beet, sunflower, sweet potatoes, tea, tomato, walnut, and yams, as well as monocots such as banana, barley, bluegrass, date palm, fescue, field corn, garlic, millet, oat, oil palm, onion, pineapple, popcorn, rice, rye, ryegrass, sorghum, sudangrass, sugarcane, sweet com, switchgrass, timothy, and wheat. Brown seaweeds, green seaweeds, red seaweeds, and microalgae can also be used.

Thus, the methods and compositions described herein can be used with dicotyledonous plants belonging, for example, to the orders Apiales, Arecales, Aristochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucoiniales, Euphorbiales, Fabales, Fagales, Gentianales, Geraniales, Haloragales, Hamamelidales, Illiciales, Juglandales, Lamiates, Laurales, Lecythidales, Leitneriales, Linales, Magniolales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhainnales, Rosales, Rubiales, Salicales, Santales, Sapindales, Sanraceniaceae, Scrophulariales, Solanales, Trochodendrales, Theales, Umbellales, Urticales, and Violales. Thc methods and compositions described herein also can be utilized. with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycacaales, Ginkgoales, Gnetales, and Pinales.

The methods and compositions can be used over a broad range of plant species, including species from the dicot genera Amaranthus, Anacardiumn, Arachis, Bertholletia, Brassica, Calendula, Camellia, Capsicum, Carthamus, Carya, Chenopodium, Cicer, Cichorium, Cinnamomum, Citrus, Citrullus, Coffea, Corylus, Crambe, Cucumis, Cucurbita, Daucus, Dioscorea, Fragaria, Glycine, Gosvypium, Helianthus, Juglans, Lactuca, Lens, Linum, Lycopersicon, Macadamia, Malus, Mangifera, Medicago, Mentha, Nicotiana, Ocimum, Olea, Phaseolus, Pistacia, Pisum, Prunus, Pyrus, Rosmarinus, Salvia, Sesamum, Solanum, Spinacia, Theobroma, Thymus, Trifolium, Vaccinium, Vigna, and Vitis; and the monocot genera Allium, Ananas, Asparagus, Avena, Curcuma, Elaeis, Festuca, Hordeum, Lemna, Lolium, Musa, Oryza, Panicum, Pennisetum, Phleum, Poa, Saccharum, Secale, Sorghum, Triticosecale, Triticum, and Zea.

The methods and compositions described herein also can be used with brown seaweeds, e.g., Ascophyllum nodosum, Fucus vesiculosus, Fucus serratus, Himanthalia elongata, and Undaria pinnatifida; red seaweeds, e.g., Chondrus crispus, Cracilaria verrucosa, Porphyra umbilicalis, and Palmaria palmata; green seaweeds, e.g., Enteromorpha spp. and Ulva spp.; and microalgae, e.g., Spirulina spp. (S. platensis and S. maxima) and Odontella aurita. In addition, the methods and compositions can be used with Crypthecodinium cohnii, Schizochytrium spp., and Haematococcus pluvialis.

In some embodiments, a plant is a member of the species Avena sativa, Brassica spp., Cicer arietinum, Gossypium spp., Glycine max, Hordeum vulgare, Lactuca saliva, Medicago sativa, Oryza sativa, Pennisetum glaucum, Phaseolus spp., Phleum pratense, Secale cereale, Trifolhum pratense, Triticum aestivum, and Zea mays.

### Expression of Protein-Modulating Polypeptides

The polynucleotides and recombinant vectors described herein can be used to express a protein-modulating polypeptide in a plant species of interest. The term “expression” refers to the process of converting genetic information of a polynucleotide into RNA through transcription, which is catalyzed by an enzyme, RNA polymerase, and into protein, through translation of mRNA on ribosomes. “Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

The polynucleotides and recombinant vectors described herein can be used to inhibit expression of a protein-modulating polypeptide in a plant species of interest. A number of nucleic acid based methods, including antisense RNA, ribozyme directed RNA cleavage, post-transcriptional gene silencing (PTGS), e.g., RNA interference (RNAi), and transcriptional gene silencing (TGS) can be used to inhibit gene expression in plants. Antisense technology is one well-known method. In this method, a nucleic acid segment from a gene to bc repressed is cloned and operably linked to a regulatory region and a transcription termination sequence so that the antisense strand of RNA is transcribed. The recombinant vector is then transformed into plants, as described herein, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence of a protein-modulating polypeptide, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the protein-modulating polypeptide, and can have a length that is shorter, the same as, or longer than the corresponding length of the sense sequence. In some cases, one strand of the stem portion of a double stranded RNA comprises a sequence that is similar or identical to the 3′ or 5′ untranslated region of an mRNA encoding a protein-modulating polypeptide, and the other strand of the stem portion of the double stranded RNA comprises a sequence that is similar or identical to the sequence that is complementary to the 3′ or 5′ untranslated region, respectively, of the mRNA encoding the protein-modulating polypeptide. In other embodiments, one strand of the stem portion of a double stranded RNA comprises a sequence that is similar or identical to the sequence of an intron in the pre-mRNA encoding a protein-modulating polypeptide, and the other strand of the stem portion comprises a sequence that is similar or identical to the sequence that is complementary to the sequence of the intron in the pre-mRNA. The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron. A double stranded RNA can have zero, one, two, three, four, five, six, seven, eight, nine, ten, or more stem-loop structures. A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing regulatory regions operably linked to nucleic acid molecules in sense orientation can also be used to inhibit the expression of a gene. The transcription product can be similar or identical to the sense coding sequence of a protein-modulating polypeptide. The transcription product can also be unpolyadenylated, lack a 5′ cap structure, or contain an unsplicable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can bc a sequence that is identical or complementary to the sequence of an mRNA, the 3′ or 5′ untranslated region of an mRNA, or an intron in a pre-mRNA encoding a protein-modulating polypeptide. In some embodiments, the sense or antisense sequence is identical or complementary to a sequence of the regulatory region that drives transcription of the gene encoding a protein-modulating polypeptide. In each case, the sense sequence is the sequence that is complementary to the antisense sequence.

The sense and antisense sequences can be any length greater than about 12 nucleotides (e.g., 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence encoding a protein-modulating polypeptide described herein. The sense sequence complementary to the antisense sequence can be a sequence present within the mRNA of the protein-modulating polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or P-DNA such that the left and right T-DNA border sequences, or the left and right border-like sequences of the P-DNA, flank or are on either side of the nucleic acid. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

### Transgenic Plant Phenotypes

In some embodiments, a plant in which expression of a protein-modulating polypeptide is modulated can have increased levels of seed protein. For example, a protein-modulating polypeptide described herein can be expressed in a transgenic plant, resulting in increased levels of seed protein. The seed protein level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the seed protein level in a corresponding control plant that does not express the transgene. In some embodiments, a plant in which expression of a protein-modulating polypeptide is modulated can have decreased levels of seed protein. The seed protein level can be decreased by at least 2 percent, e.g., 2, 3, 4, 5, 10, 15, 20, 25, 30, 35, or more than 35 percent, as compared to the seed protein level in a corresponding control plant that does not express the transgene.

Plants for which modulation of levels of seed protein can be useful include, without limitation, amaranth, barley, beans, canola, coffee, cotton, edible nuts (e.g., almond, brazil nut, cashew, hazelnut, macadamia nut, peanut, pecan, pine nut, pistachio, walnut), field corn, millet, oat, oil palm, peas, popcorn, rapeseed, rice, rye, safflower, sorghum, soybean, sunflower, sweet corn, and wheat. Increases in seed protein in such plants can provide improved nutritional content in geographic locales where dietary intake of protein/amino acid is often insufficient. Decreases in seed protein in such plants can be useful in situations where seeds are not the primary plant part that is harvested for human or animal consumption.

In some embodiments, a plant in which expression of a protein-modulating polypeptide is modulated can have increased or decreased levels of protein in one or more non-seed tissues, e.g., leaf tissues, stem tissues, root or corm tissues, or fruit tissues other than seed. For example, the protein level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the protein level in a corresponding control plant that does not express the transgene. In some embodiments, a plant in which expression of a protein-modulating polypeptide is modulated can have decreased levels of protein in one or more non-seed tissues. The protein level can be decreased by at least 2 percent, e.g., 2, 3, 4, 5, 10, 15, 20, 25, 30, 35, or more than 35 percent, as compared to the protein level in a corresponding control plant that does not express the transgene.

Plants for which modulation of levels of protein in non-seed tissues can be useful include, without limitation, alfalfa, amaranth, apple, banana, barley, beans, bluegrass, broccoli, carrot, cherry, clover, coffee, fescue, field corn, grape, grapefruit, lemon, lettuce, mango, melon, millet, oat, oil palm, onion, orange, peach, peanut, pear, peas, pineapple, plum, popcorn, potato, rapeseed, rice, rye, ryegrass, safflower, sorghum, soybean, strawberry, sugarcane, sudangrass, sunflower, sweet corn, switchgrass, timothy, tomato, and wheat. Increases in non-seed protein in such plants can provide improved nutritional content in edible fruits and vegetables, or improved animal forage. Decreases in non-seed protein can provide more efficient partitioning of nitrogen to plant part(s) that are harvested for human or animal consumption.

In some embodiments, a plant in which expression of a protein-modulating polypeptide having an amino acid sequence corresponding to SEQ ID NO:107 is modulated can have modulated levels of seed oil accompanying increased levels of seed protein. The oil level can be modulated by at least 2 percent, e.g., 2, 3, 4, 5, 10, 15, 20, 25, 30, 35, or 40 percent, as compared to the oil level in a corresponding control plant that does not express the transgene.

In some embodiments, a plant in which expression of a protein-modulating polypeptide having an amino acid sequence corresponding to SEQ ID NO:83 or SEQ ID NO:95 is modulated can have decreased levels of seed oil accompanying increased levels of seed protein. The oil level can be decreased by at least 2 percent, e.g., 2, 3, 4, 5, 10, 15, 20, 25, 30, 35, or 40 percent, as compared to the oil level in a corresponding control plant that does not express the transgene.

Typically, a difference (e.g., an increase) in the amount of oil or protein in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of oil or protein is statistically significant at p<0.01, p<0.005, or p<0.001. A statistically significant difference in, for example, the amount of protein in a transgenic plant compared to the amount in cells of a control plant indicates that the recombinant nucleic acid present in the transgenic plant results in altered protein levels.

The phenotype of a transgenic plant is evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant is said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

Information that the polypeptides disclosed herein can modulate protein content can be useful in breeding of crop plants. Based on the effect of disclosed polypeptides on protein content, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in protein content. Those polymorphisms that are correlated with an alteration in protein content can be incorporated into a marker assisted breeding program to facilitate the development of lines that have a desired alteration in protein content. Typically, a polymorphism identified in such a manner is used with polymorphisms at other loci that are also correlated with a desired alteration in protein content.

Articles of Manufacture

Transgenic plants provided herein have particular uses in the agricultural and nutritional industries. For example, transgenic plants described herein can be used to make animal feed and food products, such as grains and fresh, canned, and frozen vegetables. Suitable plants with which to make such products include alfalfa, barley, beans, clover, corn, millet, oat, peas, rice, rye, soybean, timothy, and wheat. For example, soybeans can be used to make various food products, including tofu, soy flour, and soy protein concentrates and isolates. Soy protein concentrates can be used to make textured soy protein products that resemble meat products. Soy protein isolates can be added to many soy food products, such as soy sausage patties, soybean burgers, soy protein bars, powdered soy protein beverages, soy protein baby formulas, and soy protein supplements. Such products are useful to provide desired protein and caloric content in the diet.

Seeds from transgenic plants described herein can be used as is, e.g., to grow plants, or can be used to make food products, such as flour. Seeds can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package. The label can indicate that plants grown from the seeds contained within the package can produce a crop having an altered level of protein relative to corresponding control plants.

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

The following is a list of nucleic acids that were isolated from Arabidopsis thaliana plants. Ceres Clone 38311 (Lead Number 123; At1g25560; SEQ ID NO:106) is a cDNA clone that is predicted to encode a 361 amino acid transcription factor polypeptide containing B3 and AP2 domains. Ceres Clone 120446 (Lead Number 116; SEQ ID NO:80) is a cDNA clone that is predicted to encode a 107 amino acid polypeptide. Ceres Clone 11852 (Lead Number 121; At3g29170; SEQ ID NO:82) is a cDNA clone that is predicted to encode a 121 amino acid polypeptide. Ceres Clone 8166 (Lead Number 122; At3g11660; SEQ ID NO:94) is a cDNA clone that is predicted to encode a 209 amino acid harpin induced family polypeptide. Ceres Clone 109289 (SEQ ID NO:113) is a DNA clone that is predicted to encode a 300 amino acid polypeptide. Ceres Clone 19342 (SEQ ID NO:118) is a DNA clone that is predicted to encode a 337 amino acid XAP5 polypeptide. Ceres Clone 21006 (SEQ ID NO:126) is a DNA clone that is predicted to encode a 102 amino acid glutaredoxin polypeptide. Ceres Clone 2296 (SEQ ID NO:147) is a DNA clone that is predicted to encode a 235 amino acid polypeptide having a PQ loop repeat. Ceres Clone 33038 (SEQ ID NO:154) is a DNA clone that is predicted to encode a 106 amino acid polypeptide having a heavy metal associated domain. Ceres Clone 5821 (SEQ ID NO:166) is a DNA clone that is predicted to encode a 159 amino acid ubiquitin-conjugating enzyme.

Each isolated nucleic acid described above was cloned into a Ti plasmid vector, CRS 338 or CRS 311, containing a phosphinothricin acetyltransferase gene which confers Finale™ resistance to transformed plants. Constructs were made using CRS 338 that contained Ceres Clone 38311, Ceres Clone 120446, Ceres Clone 109289, Ceres Clone 19342, Ceres Clone 21006, Ceres Clone 2296, Ceres Clone 33038, or Ceres Clone 5821, each operably linked to a CaMV 35S promoter. Constructs were made using CRS 311 that contained Ceres Clone 11852 or Ceres Clone 8166, each operably linked to the 32449 promoter. Wild-type Arabidopsis thaliana ecotype Wassilewskija (Ws) plants were transformed separately with each construct. The transformations were performed essentially as described in Bechtold et al., C.R. Acad. Sci. Paris, 316:1194-1199 (1993).

Transgenic Arabidopsis lines containing Ceres Clone 38311, Ceres Clone 120446, Ceres Clone 11852, Ceres Clone 8166, Ceres Clone 109289, Ceres Clone 19342, Ceres Clone 21006, Ceres Clone 2296, Ceres Clone 33038, or Ccres Clone 5821 were designated ME01208, ME01375, ME00363, ME00365, ME00120, ME00013, ME01386, ME00074, ME00084, or ME00090, respectively. The presence of each vector containing a Ceres clone described above in the respective transgenic Arabidopsis line transformed with the vector was confirmed by Finale™ resistance, polymerase chain reaction (PCR) amplification from green leaf tissue extract, and/or sequencing of PCR products. As controls, wild-type Arabidopsis ecotype Ws plants were transformed with the empty vector CRS 338 or the empty vector CRS 311.

### Example 2

### Analysis of Protein Content in Transgenic Arabidopsis Seeds

An analytical method based on Fourier transform near-infrared (FT-NIR) spectroscopy was developed, validated, and used to perform a high-throughput screen of transgenic seed lines for alterations in seed protein content. To calibrate the FT-NIR spectroscopy method, total nitrogen elemental analysis was used as a primary method to analyze a sub-population of randomly selected transgenic seed lines. The overall percentage of nitrogen in each sample was determined. Percent nitrogen values were multiplied by a conversion factor to obtain percent total protein values. A conversion factor of 5.30 was selected based on data for cotton, sunflower, safflower, and sesame seed (Rhee, K. C., Determination of Total Nitrogen In Handbook of Food Analytical Chemistry—Water, Proteins, Enzymes, Lipids, and Carbohydrates (R. Wrolstad, et al., ed.), John Wiley and Sons, Inc., p. 105, (2005)). The same seed lines were then analyzed by FT-NIR spectroscopy, and the protein values calculated via the primary method were entered into the FT-NIR chemometrics software (Bruker Optics, Billerica, Mass.) to create a calibration curve for analysis of seed protein content by FT-NIR spectroscopy.

Elemental analysis was performed using a FlashEA 1112 NC Analyzer (Thermo Finnigan, San Jose, Calif.). To analyze total nitrogen content, 2.00±0.15 mg of dried transgenic Arabidopsis seed was weighed into a tared tin cup. The tin cup with the seed was weighed, crushed, folded in half, and placed into an autosampler slot on the FlashEA 1112 NC Analyzer (Thermo Finnigan). Matched controls were prepared in a manner identical to the experimental samples and spaced evenly throughout the batch. The first three samples in every batch were a blank (empty tin cup), a bypass, (approximately 5 mg of aspartic acid), and a standard (5.00±0.15 mg aspartic acid), respectively. Blanks were entered between every 15 experimental samples. Each sample was analyzed in triplicate.

The FlashEA 1112 NC Analyzer (Thermo Finnigan) instrument parameters were as follows: left furnace 900° C., right furnace 840° C., oven 50° C., gas flow carrier 130 mL/min., and gas flow reference 100 mL/min. The data parameter LLOD was 0.25 mg for the standard and different for other materials. The data parameter LLOQ was 3.0 mg for the standard, 1.0 mg for seed tissue, and different for other materials.

Quantification was performed using the Eager 300 software (Thermo Finnigan). Replicate percent nitrogen measurements were averaged and multiplied by a conversion factor of 5.30 to obtain percent total protein values. For results to be considered valid, the standard deviation between replicate samples was required to be less than 10%. The percent nitrogen of the aspartic acid standard was required to be within ±1.0% of the theoretical value. For a run to be declared valid, the weight of the aspartic acid (standard) was required to be between 4.85 and 5.15 mg, and the blank(s) were required to have no recorded nitrogen content.

The same seed lines that were analyzed for elemental nitrogen content were also analyzed by FT-NIR spectroscopy, and the percent total protein values determined by elemental analysis were entered into the FT-NIR chemometrics software (Bruker Optics, Billerica, Mass.) to create a calibration curve for protein content. The protein content of each seed line based on total nitrogen elemental analysis was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed protein content using the FT-NIR chemometrics software (Bruker Optics) and the protein calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with protein levels in T₂ seed that differed by more than two standard deviations from the population mean were selected for evaluation of protein levels in the T₃ generation. All events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 3

### Analysis of Oil Content in Transgenic Arabidopsis Seeds

An analytical method based on Fourier transform near-infrared (FT-NIR) spectroscopy was developed, validated, and used to perform a high-throughput screen of transgenic seed lines for alterations in seed oil content. To calibrate the FT-NIR spectroscopy method, a sub-population of transgenic seed lines was randomly selected and analyzed for oil content using a direct primary method. Fatty acid methyl ester (FAME) analysis by gas chromatography-mass spectroscopy (GC-MS) was used as the direct primary method to determine the total fatty acid content for each seed line and produce the FT-NIR spectroscopy calibration curves for oil.

To analyze seed oil content using GC-MS, seed tissue was homogenized in liquid nitrogen using a mortar and pestle to create a powder. The tissue was weighed, and 5.0±0.25 mg were transferred into a 2 mL Eppendorf tube. The exact weight of each sample was recorded. One mL of 2.5% H₂SO₄ (v/v in methanol) and 20 μL of undecanoic acid internal standard (1 mg/mL in hexane) were added to the weighed seed tissue. The tubes were incubated for two hours at 90° C. in a pre-equilibrated heating block. The samples were removed from the heating block and allowed to cool to room temperature. The contents of each Eppendorf tube were poured into a 15 mL polypropylene conical tube, and 1.5 mL of a 0.9% NaCl solution and 0.75 mL of hexane were added to each tube. The tubes were vortexed for 30 seconds and incubated at room temperature for 15 minutes. The samples were then centrifuged at 4,000 rpm for 5 minutes using a bench top centrifuge. If emulsions remained, then the centrifugation step was repeated until they were dissipated. One hundred μL of the hexane (top) layer was pipetted into a 1.5 mL autosampler vial with minimum volume insert. The samples were stored no longer than 1 week at −80° C. until they were analyzed.

Samples were analyzed using a Shimadzu QP-2010 GC-MS (Shimadzu Scientific Instruments, Columbia, Md.). The first and last sample of each batch consisted of a blank (hexane). Every fifth sample in the batch also consisted of a blank. Prior to sample analysis, a 7-point calibration curve was generated using the Supelco 37 component FAME mix (0.00004 mg/mL to 0.2 mg/mL). The injection volume was 1 μL.

The GC parameters were as follows: column oven temperature: 70° C., inject temperature: 230° C., inject mode: split, flow control mode: linear velocity, column flow: 1.0 mL/min, pressure: 53.5 mL/min, total flow: 29.0 mL/min, purge flow: 3.0 mL/min, split ratio: 25.0. The temperature gradient was as follows: 70° C. for 5 minutes, increasing to 350° C. at a rate of 5 degrees per minute, and then held at 350° C. for 1 minute. The MS parameters were as follows: ion source temperature: 200° C., interface temperature: 240° C., solvent cut time: 2 minutes, detector gain mode: relative, detector gain: 0.6 kV, threshold: 1000, group: 1, start time: 3 minutes, end time: 62 minutes, ACQ mode: scan, interval: 0.5 second, scan speed: 666 amu/sec., start M/z: 40, end M/z: 350. The instrument was tuned each time the column was cut or a new column was used.

The data were analyzed using the Shimadzu GC-MS Solutions software. Peak areas were integrated and exported to an Excel spreadsheet. Fatty acid peak areas were normalized to the internal standard, the amount of tissue weighed, and the slope of the corresponding calibration curve generated using the FAME mixture. Peak areas were also multiplied by the volume of hexane (0.75 mL) used to extract the fatty acids.

The same seed lines that were analyzed using GC-MS were also analyzed by FT-NIR spectroscopy, and the oil values determined by the GC-MS primary method were entered into the FT-NIR chemometrics software (Bruker Optics, Billerica, Mass.) to create a calibration curve for oil content. The actual oil content of each seed line analyzed using GC-MS was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed oil content using the FT-NIR chemometrics software (Bruker Optics) and the oil calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with protein levels in T₂ seed that differed by more than two standard deviations from the population mean were also analyzed to determine oil levels in the T₃ generation. Events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 4

### Results for ME01208 Events

T₂ and T₃ seed from four events and three events, respectively, of ME01208 containing Ceres Clone 38311 was analyzed for total protein content using FT-NIR spectroscopy as described in Example 2.

The protein content in T₂ seed from three events of ME01208 was significantly increased compared to the mean protein content in seed from transgenic Arabidopsis lines planted within 30 days of ME01208. As presented in Table 1, the protein content was increased to 122%, 124%, and 121% in seed from events -01, -03, and -04, respectively, compared to the population mean.

TABLE 1
Protein content (% control) in T₂ and T₃ seed from ME01208
events containing Ceres Clone 38311

| | Event-01 | Event-02 | Event-03 | Event-04 | Control |
|---|---|---|---|---|---|
| Protein | 122 | 105 | 124 | 121 | 100 ± 9* |
| content | | | | | |
| (% control) | | | | | |
| in T₂ seed | | | | | |
| p-value** | 0.02 | 0.34 | 0.01 | 0.03 | N/A |
| Protein | 106 ± 1 | 110 ± 3 | 118 | No data | 100 ± 5 |
| content | | | | | |
| (% control) | | | | | |
| in T₃ seed | | | | | |
| p-value*** | 0.02 | 0.12 | <0.01 | No data | N/A |
| No. of T₂ | 2 | 2 | 1 | 0 | 31 |
| plants | | | | | |

*Population mean of the protein content in seed from transgenic lines planted within 30 days of ME01208. Variation is presented as the standard error of the mean.
**The p-values for T₂ seed were calculated using z-scores.
***The p-values for T₃ seed were calculated using a Student's t-test.

The protein content in T₃ seed from two events of ME01208 was significantly increased compared to the protein content in corresponding control seed. As presented in Table 1, the protein content was increased to 106% and 118% in seed from events -01 and -03, respectively, compared to the protein content in control seed.

T₂ and T₃ seed from four events and three events, respectively, of ME01208 containing Ceres Clone 38311 was also analyzed for total oil content using FT-NIR spectroscopy as described in Example 3.

The oil content in T₂ seed from three events of ME01208 was significantly increased compared to the mean oil content in seed from transgenic Arabidopsis lines planted within 30 days of ME01208. As presented in Table 2, the oil content was increased to 118%, 121%, and 119% in seed from events -01, -03, and -04, respectively, compared to the population mean.

TABLE 2
Oil content (% control) in T₂ and T₃ seed from ME01208
events containing Ceres Clone 38311

| | Event-01 | Event-02 | Event-03 | Event-04 | Control |
|---|---|---|---|---|---|
| Oil content | 118 | 113 | 121 | 119 | 100 ± 8* |
| (% control) | | | | | |
| in T₂ | | | | | |
| seed | | | | | |
| p-value** | 0.03 | 0.14 | 0.01 | 0.03 | N/A |
| Oil content | 99 ± 0 | 99 ± 1 | 93 | No data | 100 ± 4 |
| (% control) | | | | | |
| in T₃ | | | | | |
| seed | | | | | |
| p-value*** | 0.08 | 0.55 | 0.15 | No data | N/A |
| No. of | 2 | 2 | 1 | 0 | 31 |
| T₂ plants | | | | | |

*Population mean of the oil content in seed from transgenic lines planted within 30 days of ME01208. Variation is presented as the standard error of the mean.
**The p-values for T₂ seed were calculated using z-scores.
***The p-values for T₃ seed were calculated using a Student's t-test.

The oil content in T₃ seed from ME01208 events was not observed to differ significantly from the oil content in corresponding control seed (Table 2).

There were no observable or statistically significant differences between T₂ ME01208 and control plants in germination, onset of flowering, rosette area, fertility, and general morphology/architecture.

### Example 5

### Results for ME01375 Events

T₂ and T₃ seed from five events of ME01375 containing Ceres Clone 120446 was analyzed for total protein content using FT-NIR spectroscopy as described in Example 2.

The protein content in T₂ seed from three events of ME01375 was significantly increased compared to the mean protein content in seed from transgenic Arabidopsis lines planted within 30 days of ME01375. As presented in Table 3, the protein content was increased to 119%, 121%, and 125% in seed from events -01, -03, and -05, respectively, compared to the population moan.

TABLE 3
Protein content (% control) in T₂ and T₃ seed
from ME01375 events containing Ceres Clone 120446

| | Event- | Event- | Event- | Event- | Event- | |
|---|---|---|---|---|---|---|
| | 01 | 02 | 03 | 04 | 05 | Control |
| Protein | 119 | 101 | 121 | 113 | 125 | 100 ± 8* |
| content | | | | | | |
| (% control) | | | | | | |
| in T₂ seed | | | | | | |
| p-value** | 0.03 | 0.40 | 0.02 | 0.12 | <0.01 | N/A |
| Protein | 124 ± 3 | 99 ± 3 | 130 | 124 ± 3 | 132 ± 4 | 100 ± 5 |
| content | | | | | | |
| (% control) | | | | | | |
| in T₃ seed | | | | | | |
| p-value*** | <0.01 | 0.59 | <0.01 | <0.01 | <0.01 | N/A |
| No. of | 3 | 3 | 1 | 4 | 3 | 31 |
| T₂ plants | | | | | | |

*Population mean of the protein content in seed from transgenic lines planted within 30 days of ME01375. Variation is presented as the standard error of the mean.
**The p-values for T₂ seed were calculated using z-scores.
***The p-values for T₃ seed were calculated using a Student's t-test.

The protein content in T₃ seed from four events of ME01375 was significantly increased compared to the protein content in corresponding control seed. As presented in Table 3, the protein content was increased to 124%, 130%, 124%, and 132% in seed from events -01, -03, -04, and -05, respectively, compared to the protein content in control seed.

T₂ and T₃ seed from five events of ME01375 containing Ceres Clone 120446 was also analyzed for total oil content using FT-NIR spectroscopy as described in Example 3. The oil content in T₂ seed from ME01375 events was not observed to differ significantly from the mean oil content in seed from transgenic Arabidopsis lines planted within 30 days of ME01375 (Table 4). The oil content in T₃ seed from one event of ME01375 was significantly decreased compared to the oil content in corresponding control seed. As presented in Table 4, the oil content was decreased to 96% in seed from event -04 compared to the oil content in control seed.

TABLE 4
Oil content (% control) in T₂ and T₃ seed
from ME01375 events containing Ceres Clone 120446

| | Event- | Event- | Event- | Event- | Event- | |
|---|---|---|---|---|---|---|
| | 01 | 02 | 03 | 04 | 05 | Control |
| Oil content | 104 | 87 | 91 | 89 | 94 | 100 ± 11* |
| (% control) | | | | | | |
| in T₂ seed | | | | | | |
| p-value** | 0.30 | 0.17 | 0.24 | 0.20 | 0.28 | N/A |
| Oil content | 96 ± 3 | 103 ± 2 | 95 | 96 ± 2 | 93 ± 6 | 100 ± 4 |
| (% control) | | | | | | |
| in T₃ seed | | | | | | |
| p-value*** | 0.14 | 0.12 | 0.26 | 0.01 | 0.16 | N/A |
| No. of | 3 | 3 | 1 | 4 | 3 | 31 |
| T₂ plants | | | | | | |

*Population mean of the oil content in seed from transgenic lines planted within 30 days of ME01375. Variation is presented as standard error of the mean.
**The p-values for T₂ seed were calculated using z-scores.
***The p-values for T₃ seed were calculated using a Student's t-test.

There were no observable or statistically significant differences between T₂ ME01375 and control plants in germination, onset of flowering, rosette area, or fertility. The general morphology/architecture appeared wild-type in all instances except for event -03, which had a significantly decreased plant size (>30% decrease; p<0.05). Events -01 and -05 had a decreased plant size (<30%; p<0.05) that was considered acceptable.

### Example 6

### Results for ME00363 Events

T₂ and T₃ seed from five events of ME00363 containing Ceres Clone 11852 was analyzed for total protein content using FT-NIR spectroscopy as described in Example 2.

The protein content in T₂ seed from three events of ME00363 was significantly increased compared to the mean protein content of seed from transgenic Arabidopsis lines planted within 30 days of ME00363. As presented in Table 5, the protein content was increased to 122%, 126%, and 124% in seed from events -01, -02, and -03, respectively, compared to the population mean.

TABLE 5
Protein content (% control) in T₂ and T₃ seed
from ME00363 events containing Ceres Clone 11852

| | Event- | Event- | Event- | Event- | Event- | |
|---|---|---|---|---|---|---|
| | 01 | 02 | 03 | 04 | 05 | Control |
| Protein | 122 | 126 | 124 | 113 | 114 | 100 ± 9* |
| content | | | | | | |
| (% control) | | | | | | |
| in T₂ seed | | | | | | |
| p-value** | 0.02 | 0.01 | 0.01 | 0.14 | 0.14 | N/A |
| Protein | 115 ± 5 | 110 ± 2 | 104 ± 3 | 104 ± 3 | 105 ± 2 | 100 ± 5 |
| content | | | | | | |
| (% control) | | | | | | |
| in T₃ seed | | | | | | |
| p-value*** | <0.01 | <0.01 | 0.02 | 0.06 | <0.01 | N/A |
| No. of | 5 | 4 | 5 | 5 | 5 | 31 |
| T₂ plants | | | | | | |

*Population mean of the protein content in seed from transgenic lines planted within 30 days of ME00363. Variation is presented as standard error of the mean.
**The p-values for T₂ seed were calculated using z-scores.
***The p-values for T₃ seed were calculated using a Student's t-test.

The protein content in T₃ seed from four events of ME00363 was significantly increased compared to the protein content in corresponding control seed. As presented in Table 5, the protein content was increased to 115%, 110%, 104%, and 105% in seed from events -01, -02, -03, and -05, respectively, compared to the protein content in control seed.

T₂ and T₃ seed from five events of ME00363 containing Ceres Clone 11852 was also analyzed for total oil content using FT-NIR spectroscopy as described in Example 3. The oil content in T₂ seed from ME00363 events was not observed to differ significantly from the mean oil content in seed from transgenic Arabidopsis lines planted within 30 days of ME00363 (Table 6).

TABLE 6
Oil content (% control) in T₂ and T₃ seed
from ME00363 events containing Ceres Clone 11852

| | Event- | Event- | Event- | Event- | Event- | |
|---|---|---|---|---|---|---|
| | 01 | 02 | 03 | 04 | 05 | Control |
| Oil content | 92 | 95 | 97 | 105 | 109 | 100 ± 7* |
| (% control) | | | | | | |
| in T₂ seed | | | | | | |
| p-value** | 0.29 | 0.41 | 0.46 | 0.38 | 0.22 | N/A |
| Oil content | 92 ± 2 | 93 ± 3 | 91 ± 4 | 96 ± 4 | 95 ± 2 | 100 ± 4 |
| (% control) | | | | | | |
| in T₃ seed | | | | | | |
| p-value*** | <0.01 | <0.01 | <0.01 | 0.08 | <0.01 | N/A |
| No. of | 5 | 4 | 5 | 5 | 5 | 31 |
| T₂ plants | | | | | | |

*Population mean of the oil content in seed from transgenic lines planted within 30 days of ME00363. Variation is presented as standard error of the mean.
**The p-values for T₂ seed were calculated using z-scores.
***The p-values for T₃ seed were calculated using a Student's t-test.

The oil content in T₃ seed from four events of ME00363 was significantly decreased compared to the oil content in corresponding control 25 seed. As presented in Table 6, the oil content was decreased to 92%, 93%, 91%, and 95% in seeds from events -01, -02, -03, and -05, respectively, compared to the oil content in control seed.

There were no observable or statistically significant differences between T₂ ML00363 and control plants in germination, onset of flowering, rosette area, fertility, and general morphology/architecture.

### Example 7

### Results for ME00365 Events

T₂ and T₃ seed from four events of ME00365 containing Ceres Clone 8166 was analyzed for total protein content using FT-NIR spectroscopy as described in Example 2.

The protein content in T₂ seed from three events of ME00365 was significantly increased compared to the mean protein content in seed from transgenic Arabidopsis lines planted within 30 days of ME00365. As presented in Table 7, the protein content was increased to 121%, 122%, and 119% in seed from events -02, -03, and -04, respectively, compared to the population mean.

TABLE 7
Protein content (% control) in T₂ and T₃ seed
from ME00365 events containing Ceres Clone 8166

| | Event-01 | Event-02 | Event-03 | Event-04 | Control |
|---|---|---|---|---|---|
| Protein | 115 | 121 | 122 | 119 | 100 ± 9* |
| content | | | | | |
| (% control) | | | | | |
| in T₂ seed | | | | | |
| p-value** | 0.11 | 0.03 | 0.03 | 0.05 | N/A |
| Protein | 105 ± 2 | 104 ± 4 | 108 ± 2 | 116 ± 2 | 100 ± 5 |
| content | | | | | |
| (% control) | | | | | |
| in T₃ seed | | | | | |
| p-value*** | <0.01 | 0.11 | <0.01 | <0.01 | N/A |
| No. of | 5 | 5 | 5 | 5 | 31 |
| T₂ plants | | | | | |

*Population mean of the protein content in seed from transgenic lines planted within 30 days of ME00365. Variation is presented as standard error of the mean.
**The p-values for T₂ seed were calculated using z-scores.
***The p-values for T₃ seed were calculated using a Student's t-test.

The protein content in T₃ seed from three events of ME00365 was significantly increased compared to the protein content in corresponding control seed. As presented in Table 7, the protein content was increased to 105%, 108%, and 116% in seed from events -01, -03, and -04, respectively, compared to the protein content in control seed.

T₂ and T₃ seed from four events of ME00365 containing Ceres Clone 8166 was also analyzed for total oil content using FT-NIR spectroscopy as described in Example 3.

The oil content in T₂ seed from one event of ME00365 was significantly decreased compared to the mean oil content in seed from transgenic Arabidopsis lines planted within 30 days of ME00365. As presented in Table 8, the oil content was decreased to 84% in seed from event -03 compared to the population mean.

TABLE 8
Oil content (% control) in T₂ and T₃ seed
from ME00365 events containing Ceres Clone 8166

| | Event-01 | Event-02 | Event-03 | Event-04 | Control |
|---|---|---|---|---|---|
| Oil content | 93 | 90 | 84 | 87 | 100 ± 7* |
| (% control) | | | | | |
| in T₂ seed | | | | | |
| p-value** | 0.32 | 0.21 | 0.04 | 0.10 | N/A |
| Oil content | 99 ± 2 | 98 ± 2 | 96 ± 1 | 101 ± 3 | 100 ± 4 |
| (% control) | | | | | |
| in T₃ seed | | | | | |
| p-value*** | 0.31 | 0.17 | <0.01 | 0.57 | N/A |
| No. of | 5 | 5 | 5 | 5 | 31 |
| T₂ plants | | | | | |

*Population mean of the oil content in seed from transgenic lines planted within 30 days of ME00365. Variation is presented as standard error of the mean.
**The p-values for T₂ seed were calculated using z-scores.
***The p-values for T₃ seed were calculated using a Student's t-test.

The oil content in T₃ seed from one event of ME00365 was significantly decreased compared to the oil content in corresponding control seed. As presented in Table 8, the oil content was decreased to 96% in seed from event -03 compared to the oil content in control seed.

There were no observable or statistically significant differences between T₂ ME00365 and control plants in germination, onset of flowering, rosette arca, fertility, and general morphology/architecture.

### Example 8

### Results for ME00013 Events

T₂ and T₃ seed from nine events and six events, respectively, of ME00013 containing Ceres Clone 19342 was analyzed for total protein content using FT-NIR spectroscopy as described in Example 2.

The protein content in T₂ seed from three events of ME00013 was significantly increased compared to the mean protein content in seed from transgenic Arabidopsis lines planted within 30 days of ME00013. As presented in Table 9, the protein content was increased to 112%, 115%, and 119% in seed from events -04, -08, and -09, respectively, compared to the population mean.

TABLE 9
Protein content (% control) in T₂ and T₃ seed
from ME00013 events containing Ceres Clone 19342

| | Event- | Event- | Event- | Event- | Event- | Event- | Event- | Event- | Event- | |
|---|---|---|---|---|---|---|---|---|---|---|
| | 01 | 02 | 03 | 04 | 05 | 06 | 07 | 08 | 09 | Control |
| Protein | 108 | 103 | 92 | 112 | 97 | 93 | 105 | 115 | 119 | 100 ± 15* |
| content | | | | | | | | | | |
| (% | | | | | | | | | | |
| control) | | | | | | | | | | |
| in T₂ | | | | | | | | | | |
| seed | | | | | | | | | | |
| p- | 0.11 | 0.20 | 0.11 | 0.04 | 0.20 | 0.13 | 0.17 | 0.01 | <0.01 | N/A |
| value** | | | | | | | | | | |
| Protein | No | No | No | 95 ± 3 | 100 ± 2 | 103 ± 4 | 109 ± 4 | 106 ± 2 | 103 ± 7 | 100 ± 5 |
| content | data | data | data | | | | | | | |
| (% | | | | | | | | | | |
| control) | | | | | | | | | | |
| in T₃ | | | | | | | | | | |
| seed | | | | | | | | | | |
| p- | No | No | No | 0.09 | 0.88 | 0.21 | <0.01 | 0.01 | 0.39 | N/A |
| value*** | data | data | data | | | | | | | |

*Population mean of the protein content in seed from transgenic lines planted within 30 days of ME00013. Variation is presented as standard error of the mean.
**The p-values for T₂ seed were calculated using z-scores.
***The p-values for T₃ seed were calculated using a Student's t-test.

The protein content in T₃ seed from two events of ME00013 was significantly increased compared to the protein content in corresponding control seed. As presented in Table 9, the protein content was increased to 109% and 106% in seed from events -07 and -08, respectively, compared to the protein content in control seed.

### Example 9

### Results for ME00074 Events

T₂ and T₃ seed from five events of ME00074 containing Ceres Clonc 2296 was analyzed for total protein content using FT-NIR spectroscopy as described in Example 2.

The protein content in T₂ seed from two events of ME00074 was significantly increased compared to the mean protein content in seed from transgenic Arabidopsis lines planted, within 30 days of ME00074. As presented in Table 10, the protein content was increased to 114% and 115% in seed from 20 events -06 and -09, respectively, compared to the population mean.

TABLE 10
Protein content (% control) in T₂ and T₃ seed
from ME00074 events containing Ceres Clone 2296

| | Event- | Event- | Event- | Event- | Event- | |
|---|---|---|---|---|---|---|
| | 05 | 06 | 07 | 08 | 09 | Control |
| Protein | 108 | 114 | 106 | 95 | 115 | 100 ± 15* |
| content | | | | | | |
| (% control) | | | | | | |
| in T₂ seed | | | | | | |
| p-vaule** | 0.11 | 0.02 | 0.15 | 0.16 | 0.02 | N/A |
| Protein | 107 ± 6 | 110 ± 4 | 104 ± 7 | 104 ± 4 | 96 ± 4 | 100 ± 5 |
| content | | | | | | |
| (% control) | | | | | | |
| in T₃ seed | | | | | | |
| p-value*** | 0.06 | 0.01 | 0.24 | 0.14 | 0.15 | N/A |

*Population mean of the protein content in seed from transgenic lines planted within 30 days of ME00074. Variation is presented as standard error of the mean.
**The p-values for T₂ seed were calculated using z-scores.
***The p-values for T₃ seed were calculated using a Student's t-test.

The protein content in T₃ seed from one event of ME00074 was significantly increased compared to the protein content in corresponding control seed. As presented in Table 10, the protein content was increased to 110% in seed from event -06 compared to the protein content in control seed.

### Example 10

### Results for ME00084 Events

T₂ and T₃ seed from five events and two events, respectively, of ME00084 containing Ceres Clone 33038 was analyzed for total protein content using FT-NIR spectroscopy as described in Example 2.

The protein content in T₂ seed from three events of ME00084 was significantly increased compared to the mean protein content in seed from transgenic Arabidopsis lines planted within 30 days of ME00084. As presented in Table 11, the protein content was increased to 118%, 122%, and 114% in seed from events -03, -05, and -08, respectively, compared to the population mean.

TABLE 11
Protein content (% control) in T₂ and T₃ seed
from ME00084 events containing Ceres Clone 33038

| | Event- | Event- | Event- | Event- | Event- | |
|---|---|---|---|---|---|---|
| | 01 | 02 | 03 | 05 | 08 | Control |
| Protein | 102 | 106 | 118 | 122 | 114 | 100 ± 15* |
| content | | | | | | |
| (% control) | | | | | | |
| in T₂ seed | | | | | | |
| p-value** | 0.21 | 0.14 | 0.01 | <0.01 | 0.02 | N/A |
| Protein | No data | No data | 112 ± 3 | 99 ± 2 | No data | 100 ± 5 |
| content | | | | | | |
| (% control) | | | | | | |
| in T₃ seed | | | | | | |
| p-value*** | No data | No data | 0.05 | 0.31 | No data | N/A |

*Population mean of the protein content in seed from transgenic lines planted within 30 days of ME00084. Variation is presented as standard error of the mean.
**The p-values for T₂ seed were calculated using z-scores.
***The p-values for T₃ seed were calculated using a Student's t-test.

The protein content in T₃ seed from one event of ME00084 was significantly increased compared to the protein content in corresponding control seed. As presented in Table 11, the protein content was increased to 112% in seed from event -03 compared to the protein content in control seed.

### Example 11

### Results for ME00120 Events

T₂ and T₃ seed from nine events and three events, respectively, of ME00120 containing Ceres Clone 109289 was analyzed for total protein content using FT-NIR spectroscopy as described in Example 2.

The protein content in T₂ seed from two events of ME00120 was significantly increased compared to the mean protein content in seed from transgenic Arabidopsis lines planted within 30 days of ME00120. As presented in Table 12, the protein content was increased to 120% and 113% in seed from events -05 and -09, respectively, compared to the population mean.

TABLE 12
Protein content (% control) in T₂ and T₃ seed
from ME00120 events containing Ceres Clone 109289

| | Event- | Event- | Event- | Event- | Event- | Event- | Event- | Event- | Event- | |
|---|---|---|---|---|---|---|---|---|---|---|
| | 01 | 02 | 03 | 04 | 05 | 06 | 07 | 08 | 09 | Control |
| Protein | 98 | 95 | 100 | 101 | 120 | 98 | 109 | 106 | 113 | 100 ± 15* |
| content | | | | | | | | | | |
| (% | | | | | | | | | | |
| control) | | | | | | | | | | |
| in T₂ | | | | | | | | | | |
| seed | | | | | | | | | | |
| p- | 0.22 | 0.17 | 0.22 | 0.22 | <0.01 | 0.22 | 0.09 | 0.16 | 0.03 | N/A |
| value** | | | | | | | | | | |
| Protein | No | No | No | No | No | 106 ± 2 | 109 ± 2 | No | 113 ± 8 | 100 ± 5 |
| content | data | data | data | data | data | | | data | | |
| (% | | | | | | | | | | |
| control) | | | | | | | | | | |
| in T₃ | | | | | | | | | | |
| seed | | | | | | | | | | |
| p- | No | No | No | No | No | 0.12 | 0.01 | No | 0.10 | N/A |
| value*** | data | data | data | data | data | | | data | | |

*Population mean of the protein content in seed from transgenic lines planted within 30 days of ME00120. Variation is presented as standard error of the mean.
**The p-values for T₂ seed were calculated using z-scores.
***The p-values for T₃ seed were calculated using a Student's t-test.

The protein content in T₃ seed from one event of ME00120 was significantly increased compared to the protein content in corresponding control seed. As presented in Table 12, the protein content was increased to 109% in seed from event -07 compared to the protein content in control seed.

### Example 12

### Results for ME01386 Events

T₂ and T₃ seed from five events of ME01386 containing Ceres Clone 21006 was analyzed for total protein content using FT-NIR spectroscopy as described in Example 2.

The protein content in T₂ seed from four events of ME01386 was significantly increased compared to the mean protein content in seed from transgenic Arabidopsis lines planted within 30 days of ME01386. As presented in Table 13, the protein content was increased to 118%, 111%, 121%, and 116% in seed from events -01, -02, -03, and -08, respectively, compared to the population mean.

TABLE 13
Protein content (% control) in T₂ and T₃ seed
from ME01386 events containing Ceres Clone 21006

| | Event- | Event- | Event- | Event- | Event- | |
|---|---|---|---|---|---|---|
| | 01 | 02 | 03 | 04 | 08 | Control |
| Protein | 118 | 111 | 121 | 102 | 116 | 100 ± 12* |
| content | | | | | | |
| (% control) | | | | | | |
| in T₂ seed | | | | | | |
| p-value** | <0.01 | 0.05 | <0.01 | 0.25 | 0.01 | N/A |
| Protein | 125 ± 3 | 128 ± | 131 ± 1 | 119 ± 4 | 131 ± 5 | 100 ± 5 |
| content | | 10 | | | | |
| (% control) | | | | | | |
| in T₃ seed | | | | | | |
| p-value*** | <0.01 | 0.01 | <0.01 | <0.01 | <0.01 | N/A |

*Population mean of the protein content in seed from transgenic lines planted within 30 days of ME01386. Variation is presented as the standard error of the mean.
**The p-values for T₂ seed were calculated using z-scores.
***The p-values for T₃ seed were calculated using a Student's t-test.

The protein content in T₃ seed from five events of ME01386 was significantly increased compared to the protein content in corresponding control seed. As presented in Table 13, the protein content was increased to 125%, 128%, 113%, 119%, and 131% in seed from events -01, -02, -03, -04, and -08, respectively, compared to the protein content in control seed.

### Example 13

### Results for ME00090 Events

T₂ and T₃ seed from six events and three events, respectively, of ME00090 containing Ceres Clone 5821 was analyzed for total protein content using FT-NIR spectroscopy as described in Example 2.

The protein content in T₂ seed from two events of ME00090 was significantly increased compared to the mean protein content in seed from transgenic Arabidopsis lines planted within 30 days of ME00090. As presented in Table 14, the protein content was increased to 114% and 121% in seed from events -05 and -08, respectively, compared to the population mean.

TABLE 14
Protein content (% control) in T₂ and T₃ seed
from ME00090 events containing Ceres Clone 5821

| | Event- | Event- | Event- | Event- | Event- | Event- | |
|---|---|---|---|---|---|---|---|
| | 04 | 05 | 06 | 07 | 08 | 09 | Control |
| Protein | 102 | 114 | 103 | 97 | 121 | 100 | 100 ± 15* |
| content | | | | | | | |
| (% control) in | | | | | | | |
| T₂ seed | | | | | | | |
| p-value** | 0.21 | 0.02 | 0.20 | 0.20 | <0.01 | 0.22 | N/A |
| Protein | 101 ± 13 | 90 ± 1 | No | No | 123 ± 2 | No | 100 ± 5 |
| content | | | data | data | | data | |
| (% control) in | | | | | | | |
| T₃ seed | | | | | | | |
| p-value*** | 0.90 | <0.01 | No | No | <0.01 | No | N/A |
| | | | data | data | | data | |

*Population mean of the protein content in seed from transgenic lines planted within 30 days of ME00090. Variation is presented as standard error of the mean.
**The p-values for T₂ seed were calculated using z-scores.
***The p-values for T₃ seed were calculated using a Student's t-test.

The protein content in T₃ seed from one event of ME00090 was significantly increased compared to the protein content in corresponding control seed. As presented in Table 14, the protein content was increased to 123% in seed from event -08 compared to the protein content in control seed. The protein content in T₃ seed from one event of ME00090 was significantly decreased compared to the protein content in corresponding control seed. As presented in Table 14, the protein content was decreased to 90% in seed from event -05 compared to the protein content in control seed.

### Example 14

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having BLAST sequence identity of 80% or greater to the query polypeptide and an alignment length of 85% or greater along the shorter sequence in the alignment. The query polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:83, SEQ ID NO:95, SEQ ID NO:107, SEQ ID NO:114, SEQ ID NO:119, SEQ ID NO:127, SEQ ID NO:148, SEQ ID NO:155, and SEQ ID NO:167 are shown in FIGS. 1-9, respectively. The percent identities of functional homologs and/or orthologs to SEQ ID NO:83, SEQ ID NO:95, SEQ ID NO:107, SEQ ID NO:114, SEQ ID NO:119, SEQ ID NO:127, SEQ ID NO:148, SEQ ID NO:155, and SEQ ID NO:167 are shown below in Tables 15-23, respectively. The BLAST sequence identities and E-values given in Tables 15-23 were taken from the forward search round of the Reciprocal BLAST process.

TABLE 15
Percent identity to Ceres Clone 11852 (SEQ ID NO: 83)

| | | SEQ | | |
|---|---|---|---|---|
| | | ID | % | |
| Designation | Species | NO: | Identity | e-value |
| Ceres CLONE ID no. | Brassica napus | 84 | 90.8 | 5.40E−55 |
| 975428 | | | | |
| Ceres CLONE ID no. | Brassica napus | 85 | 88.3 | 1.89E−52 |
| 965227 | | | | |
| Ceres CLONE ID no. | Glycine max | 86 | 73.9 | 4.19E−39 |
| 635196 | | | | |
| Ceres | Populus balsamifera | 88 | 72.1 | 1.20E−41 |
| Annot: 1506868_PRT | subsp. trichocarpa | | | |
| Ceres CLONE ID no. | Triticum aestivum | 89 | 67.8 | 3.19E−34 |
| 891349 | | | | |
| Ceres CLONE ID no. | Triticum aestivum | 90 | 67.8 | 4.09E−34 |
| 1054465 | | | | |
| Ceres CLONE ID no. | Zea mays | 91 | 66.6 | 2.70E−37 |
| 1602143 | | | | |
| Public GI no. | Oryza sativa subsp. | 92 | 66.3 | 3.19E−34 |
| 77548568 | japonica | | | |
| Public GI no. | Oryza sativa subsp. | 93 | 60 | 4.69E−24 |
| 77553579 | japonica | | | |
| Ceres CLONE ID no. | Gossypium hirsutum | 216 | 81 | 4.50E−44 |
| 1899078 | | | | |
| Ceres CLONE ID no. | Panicum virgatum | 218 | 70.9 | 1.30E−37 |
| 1891899 | | | | |

TABLE 16
Percent identity to Ceres Clone 8166 (SEQ ID NO: 95)

| | | SEQ | | |
|---|---|---|---|---|
| | | ID | % | |
| Designation | Species | NO: | Identity | e-value |
| Ceres CLONE ID no. | Zea mays | 96 | 68.9 | 1.29E−76 |
| 1064651 | | | | |
| Ceres CLONE ID no. | Brassica napus | 97 | 67.9 | 3.10E−75 |
| 970655 | | | | |
| Ceres | Populus balsamifera | 99 | 67.4 | 8.90E−78 |
| Annot: 1475146_PRT | subsp. trichocarpa | | | |
| Ceres CLONE ID no. | Glycine max | 100 | 61.8 | 2.20E−74 |
| 465057 | | | | |
| Ceres CLONE ID no. | Glycine max | 101 | 61.1 | 2.30E−70 |
| 650444 | | | | |
| Ceres CLONE ID no. | Glycine max | 102 | 60.8 | 1.39E−70 |
| 662698 | | | | |
| Public GI no. | Oryza sativa subsp. | 103 | 47.8 | 1.60E−46 |
| 62701864 | japonica | | | |
| Ceres CLONE ID no. | Triticum aestivum | 104 | 44.1 | 1.09E−45 |
| 632710 | | | | |
| Public GI no. | Oryza sativa subsp. | 105 | 44.1 | 8.00E−45 |
| 77553726 | japonica | | | |
| Ceres CLONE ID no. | Gossypium hirsutum | 230 | 63.5 | 1.09E−72 |
| 1833556 | | | | |
| Ceres CLONE ID no. | Panicum virgatum | 232 | 46.6 | 3.70E−49 |
| 1816384 | | | | |
| Ceres CLONE ID no. | Panicum virgatum | 234 | 46.1 | 6.00E−49 |
| 1952828 | | | | |

TABLE 17
Percent identity to Ceres Clone 38311 (SEQ ID NO: 107)

| | | SEQ ID | % | |
|---|---|---|---|---|
| Designation | Species | NO: | Identity | e-value |
| Ceres CLONE ID no. | Arabidopsis | 108 | 79.7 | 2.90E−120 |
| 19561 | thaliana | | | |
| Public GI no. | Glycine max | 109 | 68.7 | 8.80E−97 |
| 72140114 | | | | |
| Public GI no. | Capsicum | 110 | 68.3 | 9.00E−101 |
| 33320073 | annuum | | | |
| Ceres CLONE ID no. | Glycine max | 111 | 67.9 | 4.49E−106 |
| 597624 | | | | |
| Public GI no. | Oryza sativa | 112 | 67.3 | 6.00E−77 |
| 34895690 | subsp. | | | |
| | japonica | | | |
| Ceres CLONE ID no. | Populus | 236 | 69.6 | 3.69E−61 |
| 1464039 | balsamifera | | | |
| | subsp. | | | |
| | trichocarpa | | | |

TABLE 18
Percent identity to Ceres Clone 109289 (SEQ ID NO: 114)

| | | SEQ ID | | |
|---|---|---|---|---|
| Designation | Species | NO: | % Identity | e-value |
| Ceres CLONE ID | Glycine max | 115 | 71.1 | 1.79E−102 |
| no. 566154 | | | | |
| Ceres CLONE ID | Glycine max | 116 | 61.8 | 4.09E−89 |
| no. 541790 | | | | |
| Ceres CLONE ID | Zea mays | 117 | 32.5 | 5.00E−12 |
| no. 218121 | | | | |

TABLE 19
Percent identity to Ceres Clone 19342 (SEQ ID NO: 119)

| | | SEQ ID | % | |
|---|---|---|---|---|
| Designation | Species | NO: | Identity | e-value |
| Ceres | Populus | 121 | 87.9 | 3.89E−155 |
| Annot: 1450498_PRT | balsamifera | | | |
| | subsp. | | | |
| | trichocarpa | | | |
| Ceres | Populus | 123 | 87.3 | 1.69E−154 |
| Annot: 1460687_PRT | balsamifera | | | |
| | subsp. | | | |
| | trichocarpa | | | |
| Ceres CLONE ID no. | Glycine max | 124 | 86.6 | 2.79E−154 |
| 1043576 | | | | |
| Public GI no. 50726581 | Oryza sativa | 125 | 84 | 7.19E−147 |
| | subsp. | | | |
| | japonica | | | |
| Ceres CLONE ID no. | Populus | 252 | 64.2 | 1.20E−96 |
| 1459859 | balsamifera | | | |
| | subsp. | | | |
| | trichocarpa | | | |

TABLE 20
Percent identity to Ceres Clone 21006 (SEQ ID NO: 127)

| | | SEQ ID | % | |
|---|---|---|---|---|
| Designation | Species | NO: | Identity | e-value |
| Ceres CLONE ID no. 1079973 | Brassica napus | 128 | 96.9 | 2.09E−46 |
| Public GI no. 7573425 | Arabidopsis thaliana | 129 | 94.9 | 1.09E−45 |
| Ceres CLONE ID no. 953083 | Brassica napus | 130 | 94.7 | 1.89E−43 |
| Ceres CLONE ID no. 1030898 | Triticum aestivum | 131 | 94.7 | 1.89E−43 |
| Ceres CLONE ID no. 940212 | Brassica napus | 132 | 92.5 | 2.00E−41 |
| Ceres CLONE ID no. 1070065 | Brassica napus | 133 | 90.5 | 3.59E−42 |
| Ceres CLONE ID no. 125679 | Arabidopsis thaliana | 134 | 84.5 | 7.69E−40 |
| Public GI no. 21537263 | Arabidopsis thaliana | 135 | 84.5 | 7.69E−40 |
| Public GI no. 24111317 | Arabidopsis thaliana | 136 | 81.1 | 5.19E−41 |
| Ceres CLONE ID no. | Arabidopsis thaliana | 137 | 81 | 3.00E−38 |
| 39560 | | | | |
| Ceres CLONE ID no. | Brassica napus | 138 | 79.5 | 1.90E−36 |
| 871147 | | | | |
| Ceres CLONE ID no. | Glycine max | 139 | 73 | 6.40E−36 |
| 510704 | | | | |
| Ceres | Populus balsamifera | 141 | 72.7 | 3.50E−35 |
| Annot: 1525141_PRT | subsp. trichocarpa | | | |
| Ccrcs | Populus balsamifera | 143 | 71.5 | 6.59E−34 |
| Annot: 1472813_PRT | subsp. trichocarpa | | | |
| Public GI no. 53748489 | Plantago major | 144 | 70.2 | 3.60E−33 |
| Public GI no. 58737210 | Oryza sativa | 145 | 61 | 1.99E−25 |
| Public GI no. 77556540 | Oryza sativa subsp. | 146 | 57.8 | 3.19E−25 |
| | japonica | | | |
| Ceres CLONE ID no. | Zea mays | 240 | 97 | 3.29E−48 |
| 1448879 | | | | |
| Ceres CLONE ID no. | Zea mays | 242 | 94.1 | 3.79E−47 |
| 1490481 | | | | |
| Ceres CLONE ID no. | Gossypium hirsutum | 244 | 70 | 6.49E−36 |
| 1856294 | | | | |
| Ceres CLONE ID no. | Gossypium hirsutum | 246 | 68 | 6.70E−34 |
| 100028679 | | | | |
| Ceres CLONE ID no. | Papaver somniferum | 248 | 66 | 6.70E−34 |
| 1629347 | | | | |
| Ceres CLONE ID no. | Panicum virgatum | 250 | 62.1 | 9.59E−26 |
| 1768062 | | | | |

TABLE 21
Percent identity to Ceres Clone 2296 (SEQ ID NO: 148)

| | | SEQ | | |
|---|---|---|---|---|
| | | ID | % | |
| Designation | Species | NO: | Identity | e-value |
| Ceres CLONE ID no. | Glycine max | 149 | 73.1 | 1.60E−71 |
| 525163 | | | | |
| Public GI no. | Oryza sativa subsp. | 150 | 71.2 | 7.59E−88 |
| 50937115 | japonica | | | |
| Ceres CLONE ID no. | Zea mays | 151 | 69.4 | 2.59E−87 |
| 242812 | | | | |
| Ceres CLONE ID no. | Zea mays | 152 | 67.8 | 2.90E−79 |
| 243125 | | | | |
| Ceres CLONE ID no. | Triticum aestivum | 153 | 67.8 | 3.39E−85 |
| 687022 | | | | |
| Ceres CLONE ID no. | Gossypium hirsutum | 238 | 78.2 | 1.40E−95 |
| 1937560 | | | | |

TABLE 22
Percent identity to Ceres Clone 33038 (SEQ ID NO: 155)

| | | SEQ ID | | |
|---|---|---|---|---|
| Designation | Species | NO: | % Identity | e-value |
| Public GI no. 18655401 | Arabidopsis thaliana | 156 | 97.1 | 8.69E−48 |
| Ceres CLONE ID no. | Brassica napus | 157 | 85.7 | 1.29E−28 |
| 1064435 | | | | |
| Ceres CLONE ID no. | Triticum aestivum | 158 | 85.5 | 2.09E−28 |
| 622673 | | | | |
| Ceres | Populus balsamifera | 160 | 85.3 | 2.69E−28 |
| Annot: 1465436_PRT | subsp. trichocarpa | | | |
| Public GI no. 47176684 | Populus alba × Populus | 161 | 85.3 | 2.69E−28 |
| | glandulosa | | | |
| Public GI no. 30039180 | Lycopersicon esculentum | 162 | 81 | 5.09E−27 |
| Ceres CLONE ID no. | Glycine max | 163 | 79 | 2.09E−21 |
| 625242 | | | | |
| Ceres CLONE ID no. | Brassica napus | 164 | 78.9 | 4.00E−27 |
| 944316 | | | | |
| Public GI no. 50942155 | Oryza sativa subsp. | 165 | 78.9 | 6.50E−27 |
| | japonica | | | |
| Ceres CLONE ID no. | Gossypium hirsutum | 254 | 85.7 | 4.00E−27 |
| 100063116 | | | | |
| Ceres CLONE ID no. | Panicum virgatum | 256 | 82.1 | 1.39E−26 |
| 1771295 | | | | |
| Ceres CLONE ID no. | Parthenium argentatum | 258 | 78.9 | 8.39E−27 |
| 1609456 | | | | |

TABLE 23
Percent identity to Ceres Clone 5821 (SEQ ID NO: 167)

| | | SEQ ID | | |
|---|---|---|---|---|
| Designation | Species | NO: | % Identity | e-value |
| Public GI no. | Arabidopsis thaliana | 168 | 98.7 | 4.49E−83 |
| 28827264 | | | | |
| Public GI no. | Arabidopsis thaliana | 169 | 86.7 | 2.00E−71 |
| 20259984 | | | | |
| Public GI no. | Arachis hypogaea | 170 | 78.6 | 2.49E−66 |
| 71040677 | | | | |
| Ceres CLONE ID no. | Glycine max | 171 | 77.9 | 8.39E−66 |
| 540991 | | | | |
| Public GI no. | Oryza sativa subsp. | 172 | 72 | 1.99E−57 |
| 50918253 | japonica | | | |
| Ceres CLONE ID no. | Triticum aestivum | 173 | 71.8 | 3.50E−60 |
| 616699 | | | | |
| Ceres CLONE ID no. | Triticum aestivum | 174 | 71.6 | 4.39E−60 |
| 677401 | | | | |
| Ceres CLONE ID no. | Zea mays | 175 | 71.25 | 2.20E−58 |
| 220463 | | | | |
| Ceres CLONE ID no. | Brassica napus | 220 | 86.7 | 8.80E−71 |
| 980825 | | | | |
| Ccrcs CLONE ID no. | Gossypium hirsutum | 222 | 78.6 | 7.39E−67 |
| 1850191 | | | | |
| Ceres CLONE ID no. | Gossypium hirsutum | 224 | 78.4 | 2.49E−66 |
| 1838128 | | | | |
| Ceres CLONE ID no. | Populus balsamifera subsp. | 226 | 77.3 | 6.59E−66 |
| 1512371 | trichocarpa | | | |
| Ceres CLONE ID no. | Panicum virgatum | 228 | 71 | 1.70E−58 |
| 1767429 | | | | |

### Example 15

### Transgenic Plants containing Homologs and/or Orthologs

Cloned sequences of some of the functional homologs and/or orthologs of protein-modulating polypeptides that were identified as outlined in Example 14 were used to make transgenic plants.

Ceres Clone 19561 (SEQ ID NO:188) is a cDNA clone isolated from Arabidopsis that encodes a functional homologue of SEQ ID NO:107, and is predicted to encode a 315 amino acid transcription factor polypeptide containing B3 and AP2 domains. Ceres Clone 39560 (SEQ ID NO:200) is a cDNA clone isolated from Arabidopsis that encodes a functional homologue of SEQ ID NO:127, and is predicted to encode a 96 amino acid glutaredoxin polypeptide.

A construct was made using the CRS 311 vector that contained Ceres Clone 19561 operably linked to the 32449 promoter. A construct was made using the CRS 338 vector that contained Ceres Clone 39560 operably linked to a CaMV 35S promoter. Wild-type Arabidopsis thaliana ecotype Wassilewskija (Ws) plants were transformed separately with each construct as described in Example 1.

Transgenic Arabidopsis lines containing Ceres Clonc 19561 or Ceres Clone 39560 were designated ME03437 or ME04801, respectively. The presence of each vector containing a Ceres clone described above in the respective transgenic Arabidopsis line transformed with the vector was confirmed by Finate™ resistance, polymerase chain reaction (PCR) amplification from green leaf tissue extract, and/or sequencing of PCR products. As controls, wild-type Arabidopsis ecotype Ws plants were transformed with the empty vector CRS 338 or the empty vector CRS 311.

### Example 16

### Results for Transgenic Plants Containing Homologs, and/or Orthologs

T₂ seed from five events of ME03437 containing Ceres Clone 39560 was analyzed for total protein content using FT-NIR spectroscopy as described in Example 2.

The protein content in T₂ seed from four events of ME03437 was modulated compared to the mean protein content in seed from transgenic Arabidopsis lines planted within 30 days of ME03437. As presented in Table 24, the protein content was increased to 102% and 106% in seed from events -01 and -05, respectively, compared to the population mean, while the protein content was decreased to 75% and 85% of the population mean in events -02 and -03, respectively.

TABLE 24
Protein content (% control) in T₂ seed
from ME03437 events containing Ceres Clone 39560

| | Event- | Event- | Event- | Event- | | |
|---|---|---|---|---|---|---|
| | 01 | 02 | 03 | 04 | Event-05 | Control |
| Protein | 102 | 75 | 85 | 100 | 106 | 100 ± 9* |
| content (% | | | | | | |
| control) in | | | | | | |
| T₂ seed | | | | | | |
| p-value** | 0.22 | <0.01 | 0.09 | 0.3 | 0.11 | N/A |

*Population mean of the protein content in seed from transgenic lines planted within 30 days of ME03437. Variation is presented as standard error of the mean.
**The p-values for T₂ seed were calculated using z-scores.

T₂ seed from four events of ME04801 containing Ceres Clone 19561 was analyzed for total protein content using FT-NIR spectroscopy as described in Example 2.

The protein content in T₂ seed from four events of ME04801 was increased compared to the mean protein content in seed from transgenic Arabidopsis lines planted within 30 days of ME04801. As presented in Table 25, the protein content was increased to 104%, 108%, 104%, and 111% in seed from events -01, -02, -04, and -05, respectively, compared to the population mean.

TABLE 25
Protein content (% control) in T₂ seed
from ME04801 events containing Ceres Clone 19561

| | Event-01 | Event-02 | Event-04 | Event-05 | Control |
|---|---|---|---|---|---|
| Protein | 104 | 108 | 104 | 111 | 100 ± 14* |
| content (% | | | | | |
| control) in | | | | | |
| T₂ seed | | | | | |
| p-value** | 0.28 | 0.20 | 0.28 | 0.14 | N/A |

*Population mean of the protein content in seed from transgenic lines planted within 30 days of ME04801. Variation is presented as standard error of the mean.
**The p-values for T₂ seed were calculated using z-scores.

Transgenic plants containing cloned sequences of some of the other functional homologs and/or orthologs of Example 14 were analyzed for total oil content in seeds by FT-NIR spectroscopy. The results were inconclusive.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modulating the level of protein in a plant, said method comprising introducing into a plant cell an isolated nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228, wherein a tissue of a plant produced from said plant cell has a difference in the level of protein as compared to the corresponding level in tissue of a control plant that does not comprise said nucleic acid.

2. The method of claim 1, said polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, and SEQ ID NOs:173-175.

3. The method of claim 1, said polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, and SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228.

4. The method of claim 1, wherein said sequence identity is 85 percent or greater.

5. The method of claim 4, wherein said sequence identity is 90 percent or greater.

6. The method of claim 4, wherein said sequence identity is 95 percent or greater.

7. The method of claim 1, wherein said nucleotide sequence encodes a polypeptide comprising an amino acid sequence selected from the group consisting of SEQ ID NO:81, SEQ ID NO:83, SEQ ID NO:95, SEQ ID NO:107, SEQ ID NO:114, SEQ ID NO:119, SEQ ID NO:127, SEQ ID NO:148, SEQ ID NO:155, and SEQ ID NO:167.

8. The method of claim 1, wherein said difference is an increase in the level of protein.

9. The method of claim 1, wherein said isolated nucleic acid is operably linked to a regulatory region.

10. The method of claim 9, wherein said regulatory region is a tissue-preferential regulatory region.

11. The method of claim 10, wherein said tissue-preferential regulatory region is a promoter.

12. The method of claim 9, wherein said regulatory region is a broadly expressing promoter.

13. The method of claim 1, wherein said plant is a dicot.

14. The method of claim 13, wherein said plant is a member of the genus Arachis, Brassica, Carthamus, Glycine, Gossypium, Helianthus, Lactuca, Linum, Lycopersicon, Medicago, Olea, Pisum, Solanum, Trifolium, or Vitis.

15. The method of claim 1, wherein said plant is a monocot.

16. The method of claim 15, wherein said plant is a member of the genus Avena, Elaeis, Hordeum, Musa, Oryza, Panicum, Phleum, Secale, Sorghum, Triticosecale, Triticum, or Zea.

17. The method of claim 1, wherein said tissue is seed tissue.

18. A method of producing a plant tissue, said method comprising growing a plant cell comprising an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228, wherein said tissue has a difference in the level of protein as compared to the corresponding level in tissue of a control plant that does not comprise said nucleic acid.

19. The method of claim 18, said polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228.

20. The method of claim 18, said polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:111, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228.

21. The method of claim 18, wherein said sequence identity is 85 percent or greater.

22. The method of claim 21, wherein said sequence identity is 90 percent or greater.

23. The method of claim 21, wherein said sequence identity is 95 percent or greater.

24. The method of claim 18, wherein said nucleotide sequence encodes a polypeptide comprising an amino acid sequence selected from the group consisting of SEQ ID NO:81 SEQ ID NO:83, SEQ ID NO:95, SEQ ID NO:107, SEQ ID NO:114, SEQ ID NO:119, SEQ ID NO:127, SEQ ID NO:148, SEQ ID NO:155, and SEQ ID NO:167.

25. The method of claim 18, wherein said difference is an increase in the level of protein.

26. The method of claim 18, wherein said exogenous nucleic acid is operably linked to a regulatory region.

27. The method of claim 26, wherein said regulatory region is a tissue-preferential regulatory region.

28. The method of claim 27, wherein said tissue-preferential regulatory region is a promoter.

29. The method of claim 26, wherein said regulatory region is a broadly expressing promoter.

30. The method of claim 18, wherein said plant tissue is dicotyledonous.

31. The method of claim 30, wherein said plant tissue is a member of the genus Arachis, Brassica, Carthamus, Glycine, Gossypium, Helianthus, Lactuca, Linum, Lycopersicon, Medicago, Olea, Pisum, Solanum, Trifolium, or Vitis.

32. The method of claim 18, wherein said plant tissue is monocotyledonous.

33. The method of claim 32, wherein said plant tissue is a member of the genus Avena, Elaeis, Hordeum, Musa, Oryza, Panicum, Phleum, Secale, Sorghum, Triticosecale, Triticum, or Zea.

34. The method of claim 18, wherein said tissue is seed tissue.

35. A plant cell comprising an exogenous nucleic acid comprising a nucleotide sequence encoding a polypeptide having 80 percent or greater sequence identity to an amino acid sequence selected from the group consisting of SEQ ID NO:81, SEQ ID NOs:83-86, SEQ ID NOs:88-93, SEQ ID NOs:95-97, SEQ ID NOs:99-105, SEQ ID NOs:107-112, SEQ ID NOs:114-117, SEQ ID NO:119, SEQ ID NO:121, SEQ ID NOs:123-125, SEQ ID NOs:127-139, SEQ ID NO:141, SEQ ID NOs:143-146, SEQ ID NOs:148-153, SEQ ID NOs:155-158, SEQ ID NOs:160-165, SEQ ID NOs:167-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228, wherein a tissue of a plant produced from said plant cell has a difference in the level of protein as compared to the corresponding level in tissue of a control plant that does not comprise said nucleic acid.

36. The plant cell of claim 35, said polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102,
SEQ ID NO:104, SEQ ID NOs:107-108, SEQ ID NO:11, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228.

37. The plant cell of claim 35, said polypeptide having 80 percent or greater sequence identity to an amino acid sequence selected from the group consisting of SEQ ID NO:81, SEQ ID NOs:83-86, SEQ ID NOs:88-91, SEQ ID NOs:95-97, SEQ ID NOs:99-102, SEQ ID NO:104, SEQ ID NOs:107-108, SEQ ID NO:11, SEQ ID NOs:114-117, SEQ ID NO:119, SEQ ID NO:121, SEQ ID NOs:123-124, SEQ ID NOs:127-128, SEQ ID NOs:130-134, SEQ ID NOs:137-139, SEQ ID NO:141, SEQ ID NO:143, SEQ ID NOs:148-149, SEQ ID NOs:151-153, SEQ ID NO:155, SEQ ID NOs:157-158, SEQ ID NO:160, SEQ ID NOs:163-164, SEQ ID NO:167, SEQ ID NO:171, SEQ ID NOs:173-175, SEQ ID NO:216, SEQ ID NO:218, SEQ ID NO:230, SEQ ID NO:232, SEQ ID NO:234, SEQ ID NO:236, SEQ ID NO:252, SEQ ID NO:240, SEQ ID NO:242, SEQ ID NO:244, SEQ ID NO:246, SEQ ID NO:248, SEQ ID NO:250, SEQ ID NO:238, SEQ ID NO:254, SEQ ID NO:256, SEQ ID NO:258, SEQ ID NO:220, SEQ ID NO:222, SEQ ID NO:224, SEQ ID NO:226, and SEQ ID NO:228.

38. The plant cell of claim 35, wherein said sequence identity is 85 percent or greater.

39. The plant cell of claim 38, wherein said sequence identity is 90 percent or greater.

40. The plant cell of claim 38, wherein said sequence identity is 95 percent or greater.

41. The plant cell of claim 35, wherein said nucleotide sequence encodes a polypeptide comprising an amino acid sequence selected from the group consisting of SEQ ID NO:81 SEQ ID NO:83, SEQ ID NO:95, SEQ ID NO:107, SEQ ID NO:114, SEQ ID NO:119, SEQ ID NO:127, SEQ ID NO:148, SEQ ID NO:155, and SEQ ID NO:167.

42. The plant cell of claim 35, wherein said difference is an increase in the level of protein.

43. The plant cell of claim 35, wherein said exogenous nucleic acid is operably linked to a regulatory region.

44. The plant cell of claim 43, wherein said regulatory region is a tissue-preferential regulatory region.

45. The plant cell of claim 44, wherein said tissue-preferential regulatory region is a promoter.

46. The plant cell of claim 43, wherein said regulatory region is a broadly expressing promoter.

47. The plant cell of claim 35, wherein said plant is a dicot.

48. The plant cell of claim 47, wherein said plant is a member of the genus Arachis, Brassica, Carthamus, Glycine, Gossypium, Helianthus, Lactuca, Linum, Lycopersicon, Medicago, Olea, Pisum, Solanum, Trifolium, or Vitis.

49. The plant cell of claim 35, wherein said plant is a monocot.

50. The plant cell of claim 49, wherein said plant is a member of the genus Avena, Elaeis, Hordeum, Musa, Oryza, Panicum, Phleum, Secale, Sorghum, Triticosecale, Triticum, or Zea.

51. The plant cell of claim 35, wherein said tissue is seed tissue.

52. A transgenic plant comprising the plant cell of claim 35.

53. Progeny of the plant of claim 52, wherein said progeny has a difference in the level of protein as compared to the level of protein in a corresponding control plant that does not comprise said isolated nucleic acid.

54. Seed from a transgenic plant according to claim 52.

55. Vegetative tissue from a transgenic plant according to claim 52.

56. A food product comprising seed or vegetative tissue from a transgenic plant according to claim 52.

57. A feed product comprising seed or vegetative tissue from a transgenic plant according to claim 52.

58. Protein from a transgenic plant according to claim 52.

59. The protein of claim 58, wherein said plant is soybean.

60. An isolated nucleic acid comprising a nucleotide sequence having 95% or greater sequence identity to a nucleotide sequence selected from the group consisting of SEQ ID NO:87, SEQ ID NO:98, SEQ ID NO:120, SEQ ID NO:122, SEQ ID NO:140, SEQ ID NO:142, SEQ ID NO:159, SEQ ID NO:215, SEQ ID NO:217, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:243, SEQ ID NO:245, SEQ ID NO:249, SEQ ID NO:251, SEQ ID NO:253, SEQ ID NO:255, SEQ ID NO:274, SEQ ID NO:275, SEQ ID NO:276, SEQ ID NO:277, SEQ ID NO:278, and SEQ ID NO:279.

61. An isolated nucleic acid comprising a nucleotide sequence encoding a polypeptide having 80% or greater sequence identity to an amino acid sequence selected from the group consisting of SEQ ID NO:88, SEQ ID NO:99, SEQ ID NO:121, SEQ ID NO:123, SEQ ID NO:141, SEQ ID NO:143, SEQ ID NO:160, SEQ ID NO:216, SEQ ID NO:218, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:244, SEQ ID NO:246, SEQ ID NO:250, SEQ ID NO:252, SEQ ID NO:254, and SEQ ID NO:256.
